SEQUENCE LISTING (71 sequences)

SEQ ID NO: 1 - DNA, 2565 bp, Homo sapiens
ccacgcgtccgggccccagggctcagcttccgccttcggcttccccttctgccaagagccctgagccactcacagcacgaccagagaacaggcctgtctcaggcaggccctgcgcctcctatgcggagatgctactgccactgctgctgtcctcgctgctgggcgggtcccaggctatggatgggagattctggatacgagtgcaggagtcagtgatggtgccggagggcctgtgcatctctgtgccctgctctttctcctacccccgacaagactggacagggtctaccccagcttatggctactggttcaaagcagtgactgagacaaccaagggtgctcctgtggccacaaaccaccagagtcgagaggtggaaatgagcacccggggccgattccagctcactggggatcccgccaaggggaactgctccttggtgatcagagacgcgcagatgcaggatgagtcacagtacttctttcgggtggagagaggaagctatgtgagatataatttcatgaacgatgggttctttctaaaagtaacagtgctcagcttcacgcccagaccccaggaccacaacaccgacctcacctgccatgtggacttctccagaaagggtgtgagcgcacagaggaccgtccgactccgtgtggcctatgcccccagagaccttgttatcagcatttcacgtgacaacacgccagccctggagccccagccccagggaaatgtcccatacctggaagcccaaaaaggccagttcctgcggctcctctgtgctgctgacagccagccccctgccacactgagctgggtcctgcagaacagagtcctctcctcgtcccatccctggggccctagacccctggggctggagctgcccggggtgaaggctggggattcagggcgctacacctgccgagcggagaacaggcttggctcccagcagcgagccctggacctctctgtgcagtatcctccagagaacctgagagtgatggtttcccaagcaaacaggacagtcctggaaaaccttgggaacggcacgtctctcccagtactggagggccaaagcctgtgcctggtctgtgtcacacacagcagccccccagccaggctgagctggacccagaggggacaggttctgagcccctcccagccctcagaccccggggtcctggagctgcctcgggttcaagtggagcacgaaggagagttcacctgccacgctcggcacccactgggctcccagcacgtctctctcagcctctccgtgcactataagaagggactcatctcaacggcattctccaacggagcgtttctgggaatcggcatcacggctcttcttttcctctgcctggccctgatcatcatgaagattctaccgaagagacggactcagacagaaaccccgaggcccaggttctcccggcacagcacgatcctggattacatcaatgtggtcccgacggctggccccctggctcagaagcggaatcagaaagccacaccaaacagtcctcggacccctcttccaccaggtgctccctccccagaatcaaagaagaaccagaaaaagcagtatcagttgcccagtttcccagaacccaaatcatccactcaagccccagaatcccaggagagccaagaggagctccattatgccacgctcaacttcccaggcgtcagacccaggcctgaggcccggatgcccaagggcacccaggcggattatgcagaagtcaagttccaatgagggtctcttaggctttaggactgggacttcggctagggaggaaggtagagtaagaggttgaagataacagagtgcaaagtttccttctctccctctctctctctctttctctctctctctctctttctctctcttttaaaaaaacatctggccagggcacagtggctcacgcctgtaatcccagcactttgggaggttgaggtgggcagatcgcctgaggtcgggagttcgagaccagcctggccaacttggtgaaaccccatctctacaaaaaatacaaaacatagctgggcttggtggtgtgtgcctgtagtcccagctgtcagacatttaaaccagagcaactccatctggaataggagctgaataaaatgaggctgagacctactgggctgcattctcagacagtggaggcattctaagtcacaggatgagacaggaggtccgtacaagatacaggtcataaagactttgctgataaaacagattgcagtaaagaagccaaccaaatcccaccaaaaccaagttggccacgagagtgacctctggtcgtcctcactgctacactcctgacagcaccatgacagtttacaaatgccatggcaacatcaggaagttacccgatatgtcccaaaagggggaggaatgaataatccaccccttgtttagcaaataagcaagaaataaccataaaagtgggcaaccagcagctctaggcgctgctcttgtctatggagtagccattcttttgttcctttactttcttaataaacttgctttcaccttaaaaaaaaaaaaaaag

SEQ ID NO: 2 - DNA, 2954 bp, Homo sapiens
tggatgggagattctggatacgagtgcaggagtcagtgatggtgccggagggcctgtgcatctctgtgccctgctctttctcctacccccgacaagactggacagggtctaccccagcttatggctactggttcaaagcagtgactgagacaaccaagggtgctcctgtggccacaaaccaccagagtcgagaggtggaaatgagcacccggggccgattccagctcactggggatcccgccaaggggaactgctccttggtgatcagagacgcgcagatgcaggatgagtcacagtacttctttcgggtggagagaggaagctatgtgagatataatttcatgaacgatgggttctttctaaaagtaacagtgctcagcttcacgcccagaccccaggaccacaacaccgacctcacctgccatgtggacttctccagaaagggtgtgagcgcacagaggaccgtccgactccgtgtggcctatgcccccagagaccttgttatcagcatttcacgtgacaacacgccagccctggagccccagccccagggaaatgtcccatacctggaagcccaaaaaggccagttcctgcggctcctctgtgctgctgacagccagccccctgccacactgagctgggtcctgcagaacagagtcctctcctcgtcccatccctggggccctagacccctggggctggagctgcccggggtgaaggctggggattcagggcgctacacctgccgagcggagaacaggcttggctcccagcagcgagccctggacctctctgtgcagtatcctccagagaacctgagagtgatggtttcccaagcaaacaggacagtcctggaaaaccttgggaacggcacgtctctcccagtactggagggccaaagcctgtgcctggtctgtgtcacacacagcagccccccagccaggctgagctggacccagaggggacaggttctgagcccctcccagccctcagaccccggggtcctggagctgcctcgggttcaagtggagcacgaaggagagttcacctgccacgctcggcacccactgggctcccagcacgtctctctcagcctctccgtgcactactccccgaagctgctgggcccctcctgctcctgggaggctgagggtctgcactgcagctgctcctcccaggccagcccggccccctctctgcgctggtggcttggggaggagctgctggaggggaacagcagccaggactccttcgaggtcacccccagctcagccgggccctgggccaacagctccctgagcctccatggagggctcagctccggcctcaggctccgctgtgaggcctggaacgtccatggggcccagagtggatccatcctgcagctgccagataagaagggactcatctcaacggcattctccaacggagcgtttctgggaatcggcatcacggctcttcttttcctctgcctggccctgatcatcatgaagattctaccgaagagacggactcagacagaaaccccgaggcccaggttctcccggcacagcacgatcctggattacatcaatgtggtcccgacggctggccccctggctcagaagcggaatcagaaagccacaccaaacagtcctcggacccctcttccaccaggtgctccctccccagaatcaaagaagaaccagaaaaagcagtatcagttgcccagtttcccagaacccaaatcatccactcaagccccagaatcccaggagagccaagaggagctccattatgccacgctcaacttcccaggcgtcagacccaggcctgaggcccggatgcccaagggcacccaggcggattatgcagaagtcaagttccaatgagggtctcttaggctttaggactgggacttcggctagggaggaaggtagagtaagaggttgaagataacagagtgcaaagtttccttctctccctctctctctctctttctctctctctctctctttctctctcttttaaaaaaacatctggccagggcacagtggctcactcctgtaatcccagcactttgggaggttgaggtgggcagatcgcctgaggtcgggagttcgagaccagcctggccaacttggtgaaaccccgtctctactaaaaatacaaaaattagctgggcatggtggcaggcgcctgtaatcctacctacttgggaagctgaggcaggagaatcacttgaacctgggagacggaggttgcagtgagccaagatcacaccattgcacgccagcctgggcaacaaagcgagactccatctcaaaaaaaaaatcctccaaatgggttgggtgtctgtaatcccagcactttgggaggctaaggtgggtggattgcttgagcccaggagttcgagaccagcctgggcaacatggtgaaaccccatctctacaaaaaatacaaaacatagctgggcttggtggtgtgtgcctgtagtcccagctgtcagacatttaaaccagagcaactccatctggaataggagctgaataaaatgaggctgagacctactgggctgcattctcagacagtggaggcattctaagtcacaggatgagacaggaggtccgtacaagatacaggtcataaagactttgctgataaaacagattgcagtaaagaagccaaccaaatcccaccaaaaccaagttggccacgagagtgacctctggtcgtcctcactgctacactcctgacagcaccatgacagtttacaaatgccatggcaacatcaggaagttacccgatatgtcccaaaagggggaggaatgaataatccaccccttgtttagcaaataagcaagaaataaccataaaagtgggcaaccagcagctctaggcgctgctcttgtctatggagtagccattcttttgttcctttactttcttaataaacttgctttcaccttaaaaaa

SEQ ID NO: 3 - DNA, 2823 bp, Homo sapiens
ccacgcgtccgggaagctatgtgagatataatttcatgaacgatgggttctttctaaaagtaacagccctgactcagaagcctgatgtctacatccccgagaccctggagcccgggcagccggtgacggtcatctgtgtgtttaactgggcctttgaggaatgtccacccccttctttctcctggacgggggctgccctctcctcccaaggaaccaaaccaacgacctcccacttctcagtgctcagcttcacgcccagaccccaggaccacaacaccgacctcacctgccatgtggacttctccagaaagggtgtgagcgtacagaggaccgtccgactccgtgtggcctatgcccccagagaccttgttatcagcatttcacgtgacaacacgccagccctggagccccagccccagggaaatgtcccatacctggaagcccaaaaaggccagttcctgcggctcctctgtgctgctgacagccagccccctgccacactgagctgggtcctgcagaacagagtcctctcctcgtcccatccctggggccctagacccctggggctggagctgcccggggtgaaggctggggattcagggcgctacacctgccgagcggagaacaggcttggctcccagcagcgagccctggacctctctgtgcagtatcctccagagaacctgagagtgatggtttcccaagcaaacaggacagtcctggaaaaccttgggaacggcacgtctctcccagtactggagggccaaagcctgtgcctggtctgtgtcacacacagcagccccccagccaggctgagctggacccagaggggacaggttctgagcccctcccagccctcagaccccggggtcctggagctgcctcgggttcaagtggagcacgaaggagagttcacctgccacgctcggcacccactgggctcccagcacgtctctctcagcctctccgtgcactactccccgaagctgctgggcccctcctgctcctgggaggctgagggtctgcactgcagctgctcctcccaggccagcccggccccctctctgcgctggtggcttggggaggagctgctggaggggaacagcagccaggactccttcgaggtcacccccagctcagccgggccctgggccaacagctccctgagcctccatggagggctcagctccggcctcaggctccgctgtgaggcctggaacgtccatggggcccagagtggatccatcctgcagctgccagataagaagggactcatctcaacggcattctccaacggagcgtttctgggaatcggcatcacggctcttcttttcctctgcctggccctgatcatcatgaagattctaccgaagagacggactcagacagaaaccccgaggcccaggttctcccggcacagcacgatcctggattacatcaatgtggtcccgacggctggccccctggctcagaagcggaatcagaaagccacaccaaacagtcctcggacccctcttccaccaggtgctccctccccagaatcaaagaagaaccagaaaaagcagtatcagttgcccagtttcccagaacccaaatcatccactcaagccccagaatcccaggagagccaagaggagctccattatgccacgctcaacttcccaggcgtcagacccaggcctgaggcccggatgcccaagggcacccaggcggattatgcagaagtcaagttccaatgagggtctcttaggctttaggactgggacttcggctagggaggaaggtagagtaagaggttgaagataacagagtgcaaagtttccttctctccctctctctctctctttctctctctctctctctttctctctcttttaaaaaaacatctggccagggcacagtggctcacgcctgtaatcccagcactttgggaggttgaggtgggcagatcgcctgaggtcgggagttcgagaccagcctggccaacttggtgaaaccccgtctctactaaaaatacaaaaattagctgggcatggtggcaggcgcctgtaatcctacctacttgggaagctgaggcaggagaatcacttgaacctgggagacggaggttgcagtgagccaagatcacaccattgcatgccagcctgggcaacaaagcgagactccatctcaaaaaaaaaatcctccaaatgggttgggtgtctgtaatcccagcactttgggaggctaaggtgggtggattgcttgagcccaggagttcgagaccagcctgggcaacatggtgaaaccccatctctacaaaaaatacaaaacatagctgggcttggtggtgtgtgcctgtagtcccagctgtcagacatttaaaccagagcaactccatctggaataggagctgaatacaatgaggctgagacctactgggctgcattctcagacagtggaggcattctaagtcacaggatgagacaggaggtccgtacaagatacaggtcataaagactttgctgataaaacagattgcagtaaagaagccaaccaaatcccaccaaaaccaagttggccacgagagtgacctctggtcgtcctcactgctacactcctgacagcaccatgacagtttacaaatgccatggcaacatcaggaagttacccgatatgtcccaaaagggggaggaatgaataatccaccccttgtttagcaaataagcaagaaataaccataaaagtgggcaaccagcagctctaggcgctgctcttgtctatggagtagccattcttttgttcctttactttcttaataaacttgctttcaccttaaaaaaa

SEQ ID NO: 4 - DNA, 1665 bp, Homo sapiens
cccccgacaagactggacagggtctaccccagcttatggctactggttcaaagcagtgactgagacaaccaagggtgctcctgtggccacaaaccaccagagtcgagaggtggaaatgagcacccggggccgattccagctcactggggatcccgccaaggggaactgctccttggtgatcagagacgcgcagatgcaggatgagtcacagtacttctttcgggtggagagaggaagctatgtgagatataatttcatgaacgatgggttctttctaaaagtaacagccctgactcagaagcctgatgtctacatccccgagaccctggagcccgggcagccggtgacggtcatctgtgtgtttaactgggcctttgaggaatgtccacccccttctttctcctggacgggggctgccctctcctcccaaggaaccaaaccaacgacctcccacttctcagtgctcagcttcacgcccagaccccaggaccacaacaccgacctcacctgccatgtggacttctccagaaagggtgtgagcgtacagaggaccgtccgactccgtgtggcctatgcccccagagaccttgttatcagcatttcacgtgacaacacgccagccctggagccccagccccagggaaatgtcccatacctggaagcccaaaaaggccagttcctgcggctcctctgtgctgctgacagccagccccctgccacactgagctgggtcctgcagaacagagtcctctcctcgtcccatccctggggccctagacccctggggctggagctgcccggggtgaaggctggggattcagggcgctacacctgccgagcggagaacaggcttggctcccagcagcgagccctggacctctctgtgcagtatcctccagagaacctgagagtgatggtttcccaagcaaacaggacagtcctggaaaaccttgggaacggcacgtctctcccagtactggagggccaaagcctgtgcctggtctgtgtcacacacagcagccccccagccaggctgagctggacccagaggggacaggttctgagcccctcccagccctcagaccccggggtcctggagctgcctcgggttcaagtggagcacgaaggagagttcacctgccacgctcggcacccactgggctcccagcacgtctctctcagcctctccgtgcactataagaagggactcatctcaacggcattctccaacggagcgtttctgggaatcggcatcacggctcttcttttcctctgcctggccctgatcatgtaggttaagaggaggcgtggcggggtctggggcctggacccaggaagaggggaggtgttgcaggccgaaagagtgaaggtcgtgatcaacgcagtatacctctggaggttacatgagtaaacagcaaactgttctcataaatgcagaatgttgtccaactgacaaactgcgtctgcttcccagagggaatgctgagggcagtcacgccccaagcgaagtgtttcttgtaattaggcacagctgaagcttgttagtaataatatgaacctgtgatcaattaaacagctgaccaattgttaaaaaaaaaaaaaaaaaaaaaaaaaaaaaaaaaaaaaaaaaaaaaaaaaaaaaaaaaaaaaaaaaaaaaaaaaagg

SEQ ID NO: 5 - DNA, 1554 bp, Homo sapiens
ctggtgacggtgcaggagggcctgtgtgtccatgtgccctgctccttctcctacccccaggatggctggactgactctgacccagttcatggctactggttccgggcaggagacagaccataccaagacgctccagtggccacaaacaacccagacagagaagtgcaggcagagacccagggccgattccaactccttggggacatttggagcaacgactgctccctgagcatcagagacgccaggaagagggataaggggtcatatttctttcggctagagagaggaagcatgaaatggagttacaaatcacagttgaattacaaaactaagcagctgtctgtgtttgtgacagccctgacccataggcctgacatcctcatcctagggaccctagagtctggccactccaggaacctgacctgctctgtgccctgggcctgtaagcaggggacaccccccatgatctcctggattggggcctccgtgtcctccccgggccccactactgcccgctcctcagtgctcacccttaccccaaagccccaggaccacggcaccagcctcacctgtcaggtgaccttgcctgggacaggtgtgaccacgaccagtaccgtccgcctcgatgtgtcctaccctccttggaacttgaccatgactgtcttccaaggagatgccacagcatccacagccctgggaaatggctcatctctttcagtccttgagggccagtctctgcgcctggtctgtgctgtcaacagcaatccccctgccaggctgagctggacccgggggagcctgaccctgtgcccctcacggtcctcaaaccctgggctgctggagctgcctcgagtgcacgtgagggatgaaggggaattcacctgccgagctcagaacgctcagggctcccagcacatttccctgagcctctccctgcagaatgagggcacaggcacctcaagacctgtatcacaagtgacactggcagcagtcgggggagctggagccacagccctggccttcctgtccttctgcatcatcttcatcatagtgaggtcctgcgggaagaaatcggcaaggccagcagcgggcgtgggggatacaggcatggaagatgcaaaggccatcaggggctcggcctctcagggacccctgactgaatcctggaaagatggcaaccccctgaagaagcctcccccagctgttgccccctcgtcaggggaggaaggagagctccattatgcaaccctcagcttccataaagtgaagcctcaggacccgcagggacaggaggccactgacagtgaatactcggagatcaagatccacaagcgagaaactgcagagactcaggcctgtttgaggaatcacaacccctccagcaaagaagtcagaggctgattctcacagaacaagaaccctctagagccccatgctatgcaatgtgcctggttccccttccgccatgattgtaagtttcctgaggcctcccccgccatgtggaactgtgagttaattacacctctttcatttataaattaaaaaaaaaaaaaa

SEQ ID NO: 6 - DNA, 1676 bp, Homo sapiens
aacagacgttccctcgcggccctggcacctctaaccccagacatgctgctgctgctgctgcccctgctctgggggagggagagggcggaaggacagacaagtaaactgctgacgatgcagagttccgtgacggtgcaggaaggcctgtgtgtccatgtgccctgctccttctcctacccctcgcatggctggatttaccctggcccagtagttcatggctactggttccgggaaggggccaatacagaccaggatgctccagtggccacaaacaacccagctcgggcagtgtgggaggagactcgggaccgattccacctccttggggacccacataccaagaattgcaccctgagcatcagagatgccagaagaagtgatgcggggagatacttctttcgtatggagaaaggaagtataaaatggaattataaacatcaccggctctctgtgaatgtgacagccttgacccacaggcccaacatcctcatcccaggcaccctggagtccggctgcccccagaatctgacctgctctgtgccctgggcctgtgagcaggggacaccccctatgatctcctggatagggacctccgtgtcccccctggacccctccaccacccgctcctcggtgctcaccctcatcccacagccccaggaccatggcaccagcctcacctgtcaggtgaccttccctggggccagcgtgaccacgaacaagaccgtccatctcaacgtgtcctacccgcctcagaacttgaccatgactgtcttccaaggagacggcacagtatccacagtcttgggaaatggctcatctctgtcactcccagagggccagtctctgcgcctggtctgtgcagttgatgcagttgacagcaatccccctgccaggctgagcctgagctggagaggcctgaccctgtgcccctcacagccctcaaacccgggggtgctggagctgccttgggtgcacctgagggatgcagctgaattcacctgcagagctcagaaccctctcggctctcagcaggtctacctgaacgtctccctgcagagcaaagccacatcaggagtgactcagggggtggtcgggggagctggagccacagccctggtcttcctgtccttctgcgtcatcttcgttgtagtgaggtcctgcaggaagaaatcggcaaggccagcagcgggcgtgggagatacgggcatagaggatgcaaacgctgtcaggggttcagcctctcaggggcccctgactgaaccttgggcagaagacagtcccccagaccagcctcccccagcttctgcccgctcctcagtgggggaaggagagctccagtatgcatccctcagcttccagatggtgaagccttgggactcgcggggacaggaggccactgacaccgagtactcggagatcaagatccacagatgagaaactgcagagactcaccctgattgagggatcacagcccctccaggcaagggagaagtcagaggctgattcttgtagaattaacagccctcaacgtgatgagctatgataacactatgaattatgtgcagagtgaaaagcacacaggctttagagtcaaagtatctcaaacctgaatccacactgtgccctcccttttatttttttaactaaaagacagacaaattcctaaaaaaaaaaaa

SEQ ID NO: 7 - DNA, 1831 bp, Homo sapiens
gaagaaccctgaggaacagacgttccctcgcggccctggcacctctaaccccagacatgctgctgctgctgctgcccctgctctgggggagggagagggcggaaggacagacaagtaaactgctgacgatgcagagttccgtgacggtgcaggaaggcctgtgtgtccatgtgccctgctccttctcctacccctcgcatggctggatttaccctggcccagtagttcatggctactggttccgggaaggggccaatacagaccaggatgctccagtggccacaaacaacccagctcgggcagtgtgggaggagactcgggaccgattccacctccttggggacccacataccgagaattgcaccctgagcatcagagatgccagaagaagtgatgcggggagatacttctttcgtatggagaaaggaagtataaaatggaattataaacatcaccggctctctgtgaatgtgacagccttgacccacaggcccaacatcctcatcccaggcaccctggagtccggctgcccccagaatctgacctgctctgtgccctgggcctgtgagcaggggacaccccctatgatctcctggatagggacctccgtgtcccccctggacccctccaccacccgctcctcggtgctcaccctcatcccacagccccaggaccatggcaccagcctcacctgtcaggtgaccttccctggggccagcgtgaccacgaacaagaccgtccatctcaacgtgtcctgtgagtgctgggccgggacgcctgggtccctgatgggacccgcctcagaacttgaccatgactgtcttccaaggagacggcacagtatccacagtcttgggaaatggctcatctctgtcactcccagagggccagtctctgcgcctggtctgtgcagttgatgcagttgacagcaatccccctgccaggctgagcctgagctggagaggcctgaccctgtgcccctcacagccctcaaacccgggggtgctggagctgccttgggtgcacctgagggatgaagctgaattcacctgcagagctcagaaccctctcggctctcagcaggtctacctgaacgtctccctgcagagcaaagccacatcaggagtgactcagggggtggtcgggggagctggagccacagccctggtcttcctgtccttctgcgtcatcttcgttgtagtgaggtcctgcaggaagaaatcggcaaggccagcagcgggcgtgggagatacgggcatagaggatgcaaacgctgtcaggggttcagcctctcaggtgagtgatgtggactctccacagccagcatgtagcctggacacctcccacaggatgacccccaggactaatcagctgggcgtagccaaagttacctcctctctgttcttcctttcttctctgtagccccaaatcacaatgtttggttggtttcctcccctaagaacagcttttattgtctctgctccctatcctgacccttcattgctgaggcctgaggatctctgtcttttgttccctcacctgtctgcctgtctcctctcctttcctgcctggggggactgtccagaagacatcatcgtccagttcctctgcatttgaacagctgttcccccacccctcaataccgtttagagcagaagccagcaaatactatctgtcagggacagatagaaactattttcggcttcatgggccacacagtctcattgcagctcctcaaatctgctgttgtagcaagaaagaagccatataccctgtgtaaacaaatgaatatggctgtgtgccaataaaactattcacaaacataaaaaaa

SEQ ID NO: 8 - protein, 544 aa, Homo sapiens
MLLPLLLSSLLGGSQAMDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSVQYPPENLRVMVSQANRTVLENLGNGTSLPVLEGQSLCLVCVTHSSPPARLSWTQRGQVLSPSQPSDPGVLELPRVQVEHEGEFTCHARHPLGSQHVSLSLSVHYKKGLISTAFSNGAFLGIGITALLFLCLALIIMKILPKRRTQTETPRPRFSRHSTILDYINVVPTAGPLAQKRNQKATPNSPRTPLPPGAPSPESKKNQKKQYQLPSFPEPKSSTQAPESQESQEELHYATLNFPGVRPRPEARMPKGTQADYAEVKFQ

SEQ ID NO: 9 - protein, 622 aa, Homo sapiens
DGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSVQYPPENLRVMVSQANRTVLENLGNGTSLPVLEGQSLCLVCVTHSSPPARLSWTQRGQVLSPSQPSDPGVLELPRVQVEHEGEFTCHARHPLGSQHVSLSLSVHYSPKLLGPSCSWEAEGLHCSCSSQASPAPSLRWWLGEELLEGNSSQDSFEVTPSSAGPWANSSLSLHGGLSSGLRLRCEAWNVHGAQSGSILQLPDKKGLISTAFSNGAFLGIGITALLFLCLALIIMKILPKRRTQTETPRPRFSRHSTILDYINVVPTAGPLAQKRNQKATPNSPRTPLPPGAPSPESKKNQKKQYQLPSFPEPKSSTQAPESQESQEELHYATLNFPGVRPRPEARMPKGTQADYAEVKFQ

SEQ ID NO: 10 - protein, 575 aa, Homo sapiens
GSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSVQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSVQYPPENLRVMVSQANRTVLENLGNGTSLPVLEGQSLCLVCVTHSSPPARLSWTQRGQVLSPSQPSDPGVLELPRVQVEHEGEFTCHARHPLGSQHVSLSLSVHYSPKLLGPSCSWEAEGLHCSCSSQASPAPSLRWWLGEELLEGNSSQDSFEVTPSSAGPWANSSLSLHGGLSSGLRLRCEAWNVHGAQSGSILQLPDKKGLISTAFSNGAFLGIGITALLFLCLALIIMKILPKRRTQTETPRPRFSRHSTILDYINVVPTAGPLAQKRNQKATPNSPRTPLPPGAPSPESKKNQKKQYQLPSFPEPKSSTQAPESQESQEELHYATLNFPGVRPRPEARMPKGTQADYAEVKFQ

SEQ ID NO: 11 - protein, 430 aa, Homo sapiens
PRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSVQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSVQYPPENLRVMVSQANRTVLENLGNGTSLPVLEGQSLCLVCVTHSSPPARLSWTQRGQVLSPSQPSDPGVLELPRVQVEHEGEFTCHARHPLGSQHVSLSLSVHYKKGLISTAFSNGAFLGIGITALLFLCLALIM

SEQ ID NO: 12 - protein, 466 aa, Homo sapiens
LVTVQEGLCVHVPCSFSYPQDGWTDSDPVHGYWFRAGDRPYQDAPVATNNPDREVQAETQGRFQLLGDIWSNDCSLSIRDARKRDKGSYFFRLERGSMKWSYKSQLNYKTKQLSVFVTALTHRPDILILGTLESGHSRNLTCSVPWACKQGTPPMISWIGASVSSPGPTTARSSVLTLTPKPQDHGTSLTCQVTLPGTGVTTTSTVRLDVSYPPWNLTMTVFQGDATASTALGNGSSLSVLEGQSLRLVCAVNSNPPARLSWTRGSLTLCPSRSSNPGLLELPRVHVRDEGEFTCRAQNAQGSQHISLSLSLQNEGTGTSRPVSQVTLAAVGGAGATALAFLSFCIIFIIVRSCGKKSARPAAGVGDTGMEDAKAIRGSASQGPLTESWKDGNPLKKPPPAVAPSSGEEGELHYATLSFHKVKPQDPQGQEATDSEYSEIKIHKRETAETQACLRNHNPSSKEVRG

SEQ ID NO: 13 - protein, 463 aa, Homo sapiens
MLLLLLPLLWGRERAEGQTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGVVGGAGATALVFLSFCVIFVVVRSCRKKSARPAAGVGDTGIEDANAVRGSASQGPLTEPWAEDSPPDQPPPASARSSVGEGELQYASLSFQMVKPWDSRGQEATDTEYSEIKIHR

SEQ ID NO: 14 - protein, 286 aa, Homo sapiens
MLLLLLPLLWGRERAEGQTSKLLTMQSSVTVQEGLCVHVPCSFSYPSHGWIYPGPVVHGYWFREGANTDQDAPVATNNPARAVWEETRDRFHLLGDPHTENCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSCECWAGTPGSLMGPASELDHDCLPRRRHSIHSLGKWLISVTPRGPVSAPGLCS

SEQ ID NO: 15 - DNA, 2529 bp, Homo sapiens
ccacgcgtccgggccccagggctcagcttccgccttcggcttccccttctgccaagagccctgagccactcacagcacgaccagagaacaggcctgtctcaggcaggccctgcgcctcctatgcggagatgctactgccactgctgctgtcctcgctgctgggcgggtcccaggctatggatgggagattctggatacgagtgcaggagtcagtgatggtgccggagggcctgtgcatctctgtgccctgctctttctcctacccccgacaagactggacagggtctaccccagcttatggctactggttcaaagcagtgactgagacaaccaagggtgctcctgtggccacaaaccaccagagtcgagaggtggaaatgagcacccggggccgattccagctcactggggatcccgccaaggggaactgctccttggtgatcagagacgcgcagatgcaggatgagtcacagtacttctttcgggtggagagaggaagctatgtgagatataatttcatgaacgatgggttctttctaaaagtaacagccctgactcagaagcctgatgtctacatccccgagaccctggagcccgggcagccggtgacggtcatctgtgtgtttaactgggcctttgaggaatgtccacccccttctttctcctggacgggggctgccctctcctcccaaggaaccaaaccaacgacctcccacttctcagtgctcagcttcacgcccagaccccaggaccacaacaccgacctcacctgccatgtggacttctccagaaagggtgtgagcgcacagaggaccgtccgactccgtgtggcctatgcccccagagaccttgttatcagcatttcacgtgacaacacgccagccctggagccccagccccagggaaatgtcccatacctggaagcccaaaaaggccagttcctgcggctcctctgtgctgctgacagccagccccctgccacactgagctgggtcctgcagaacagagtcctctcctcgtcccatccctggggccctagacccctggggctggagctgcccggggtgaaggctggggattcagggcgctacacctgccgagcggagaacaggcttggctcccagcagcgagccctggacctctctgtgcagtatcctccagagaacctgagagtgatggtttcccaagcaaacaggacagtcctggaaaaccttgggaacggcacgtctctcccagtactggagggccaaagcctgtgcctggtctgtgtcacacacagcagccccccagccaggctgagctggacccagaggggacaggttctgagcccctcccagccctcagaccccggggtcctggagctgcctcgggttcaagtggagcacgaaggagagttcacctgccacgctcggcacccactgggctcccagcacgtctctctcagcctctccgtgcactactccccgaagctgctgggcccctcctgctcctgggaggctgagggtctgcactgcagctgctcctcccaggccagcccggccccctctctgcgctggtggcttggggaggagctgctggaggggaacagcagccaggactccttcgaggtcacccccagctcagccgggccctgggccaacagctccctgagcctccatggagggctcagctccggcctcaggctccgctgtgaggcctggaacgtccatggggcccagagtggatccatcctgcagctgccagataagaagggactcatctcagatccggagcccaaatcttgtgacaaaactcacacatgcccaccgtgcccagcacctgaattcgagggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagccccgctccccgggctctcgcggtcgcacgaggatgcttctaga

SEQ ID NO: 16 - protein, 779 aa, Artificial Sequence [sequence omitted]

SEQ ID NO: 17 - DNA, 1053 bp, Artificial Sequence
  Description of Artificial Sequence L3cyto-wt
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccccgaattccaagagacggactcagacagaaaccccgaggcccaggttctcccggcacagcacgatcctggattacatcaatgtggtcccgacggctggccccctggctcagaagcggaatcagaaagccacaccaaacagtcctcggacccctcttccaccaggtgctccctccccagaatcaaagaagaaccagaaaaagcagtatcagttgcccagtttcccagaacccaaatcatccactcaagccccagaatcccaggagagccaagaggagctccattatgccacgctcaacttcccaggcgtcagacccaggcctgaggcccggatgcccaagggcacccaggcggattatgcagaagtcaagttccaa

SEQ ID NO: 18 - DNA, 1053 bp, Artificial Sequence
  Description of Artificial Sequence L3cyto- Y641F
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccccgaattccaagagacggactcagacagaaaccccgaggcccaggttctcccggcacagcacgatcctggattacatcaatgtggtcccgacggctggccccctggctcagaagcggaatcagaaagccacaccaaacagtcctcggacccctcttccaccaggtgctccctccccagaatcaaagaagaaccagaaaaagcagtttcagttgcccagtttcccagaacccaaatcatccactcaagccccagaatcccaggagagccaagaggagctccattatgccacgctcaacttcccaggcgtcagacccaggcctgaggcccggatgcccaagggcacccaggcggattatgcagaagtcaagttccaa

SEQ ID NO: 19 - DNA, 1053 bp, Artificial Sequence
  Description of Artificial Sequence L3cyto- Y667F
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccccgaattccaagagacggactcagacagaaaccccgaggcccaggttctcccggcacagcacgatcctggattacatcaatgtggtcccgacggctggccccctggctcagaagcggaatcagaaagccacaccaaacagtcctcggacccctcttccaccaggtgctccctccccagaatcaaagaagaaccagaaaaagcagtatcagttgcccagtttcccagaacccaaatcatccactcaagccccagaatcccaggagagccaagaggagctccattttgccacgctcaacttcccaggcgtcagacccaggcctgaggcccggatgcccaagggcacccaggcggattatgcagaagtcaagttccaa

SEQ ID NO: 20 - DNA, 750 bp, Artificial Sequence
  Description of Artificial Sequence L3cyto- Y691F
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccccgaattccaagagacggactcagacagaaaccccgaggcccaggttctcccggcacagcacgatcctggat

SEQ ID NO: 21 - DNA, 894 bp, Artificial Sequence
  Description of Artificial Sequence L3cyto-Y641 alone
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccccgaattccatcaatgtggtcccgacggctggccccctggctcagaagcggaatcagaaagccacaccaaacagtcctcggacccctcttccaccaggtgctccctccccagaatcaaagaagaaccagaaaaagcagtatcagttgcccagtttcccagaacccaaatcatccactcaagccccagaatcccaggagagccaagaggagctccat

SEQ ID NO: 22 - protein, 348 aa, Artificial Sequence
  Description of Artificial Sequence L3cyto-wt
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPNSKRRTQTETPRPRFSRHSTILDYINVVPTAGPLAQKKATPNSPRTPLPPGAPSPESKKNQKKQYQLPSFPEPKSSTQAPESQESQEELHYATLNFPGVRPRPEARMPKGTQADYAEVKFQ

SEQ ID NO: 23 - protein, 348 aa, Artificial Sequence
  Description of Artificial Sequence L3cyto- Y641F
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPNSKRRTQTETPRPRFSRHSTILDYINVVPTAGPLAQKKATPNSPRTPLPPGAPSPESKKNQKKQFQLPSFPEPKSSTQAPESQESQEELHYATLNFPGVRPRPEARMPKGTQADYAEVKFQ

SEQ ID NO: 24 - protein, 348 aa, Artificial Sequence
  Description of Artificial Sequence L3cyto- Y667F
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPNSKRRTQTETPRPRFSRHSTILDYINVVPTAGPLAQKKATPNSPRTPLPPGAPSPESKKNQKKQYQLPSFPEPKSSTQAPESQESQEELHFATLNFPGVRPRPEARMPKGTQADYAEVKFQ

SEQ ID NO: 25 - protein, 348 aa, Artificial Sequence
  Description of Artificial Sequence L3cyto- Y691F
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPNSKRRTQTETPRPRFSRHSTILDYINVVPTAGPLAQKKATPNSPRTPLPPGAPSPESKKNQKKQYQLPSFPEPKSSTQAPESQESQEELHYATLNFPGVRPRPEARMPKGTQADFAEVKFQ

SEQ ID NO: 26 - protein, 298 aa, Artificial Sequence
  Description of Artificial Sequence L3cyto-Y641 alone
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPNSINVVPTAGPLAQKRNQKATPNSPRTPLPPGAPSPESKKNQKKQYQLPSFPEPKSSTQAPESQESQEELH

SEQ ID NO: 27 - DNA, 3024 bp, Artificial Sequence
  Description of Artificial Sequence Siglec-BMSL3-995-2
ccacgcgtccgggccccagggctcagcttccgccttcggcttccccttctgccaagagccctgagccactcacagcacgaccagagaacaggcctgtctcaggcaggccctgcgcctcctatgcggagatgctactgccactgctgctgtcctcgctgctgggcgggtcccaggctatggatgggagattctggatacgagtgcaggagtcagtgatggtgccggagggcctgtgcatctctgtgccctgctctttctcctacccccgacaagactggacagggtctaccccagcttatggctactggttcaaagcagtgactgagacaaccaagggtgctcctgtggccacaaaccaccagagtcgagaggtggaaatgagcacccggggccgattccagctcactggggatcccgccaaggggaactgctccttggtgatcagagacgcgcagatgcaggatgagtcacagtacttctttcgggtggagagaggaagctatgtgagatataatttcatgaacgatgggttctttctaaaagtaacagccctgactcagaagcctgatgtctacatccccgagaccctggagcccgggcagccggtgacggtcatctgtgtgtttaactgggcctttgaggaatgtccacccccttctttctcctggacgggggctgccctctcctcccaaggaaccaaaccaacgacctcccacttctcagtgctcagcttcacgcccagaccccaggaccacaacaccgacctcacctgccatgtggacttctccagaaagggtgtgagcgcacagaggaccgtccgactccgtgtggcctatgcccccagagaccttgttatcagcatttcacgtgacaacacgccagccctggagccccagccccagggaaatgtcccatacctggaagcccaaaaaggccagttcctgcggctcctctgtgctgctgacagccagccccctgccacactgagctgggtcctgcagaacagagtcctctcctcgtcccatccctggggccctagacccctggggctggagctgcccggggtgaaggctggggattcagggcgctacacctgccgagcggagaacaggcttggctcccagcagcgagccctggacctctctgtgcagtatcctccagagaacctgagagtgatggtttcccaagcaaacaggacagtcctggaaaaccttgggaacggcacgtctctcccagtactggagggccaaagcctgtgcctggtctgtgtcacacacagcagccccccagccaggctgagctggacccagaggggacaggttctgagcccctcccagccctcagaccccggggtcctggagctgcctcgggttcaagtggagcacgaaggagagttcacctgccacgctcggcacccactgggctcccagcacgtctctctcagcctctccgtgcactactccccgaagctgctgggcccctcctgctcctgggaggctgagggtctgcactgcagctgctcctcccaggccagcccggccccctctctgcgctggtggcttggggaggagctgctggaggggaacagcagccaggactccttcgaggtcacccccagctcagccgggccctgggccaacagctccctgagcctccatggagggctcagctccggcctcaggctccgctgtgaggcctggaacgtccatggggcccagagtggatccatcctgcagctgccagataagaagggactcatctcaacggcattctccaacggagcgtttctgggaatcggcatcacggctcttcttttcctctgcctggccctgatcatcatgaagattctaccgaagagacggactcagacagaaaccccgaggcccaggttctcccggcacagcacgatcctggattacatcaatgtggtcccgacggctggccccctggctcagaagcggaatcagaaagccacaccaaacagtcctcggacccctcttccaccaggtgctccctccccagaatcaaagaagaaccagaaaaagcagtatcagttgcccagtttcccagaacccaaatcatccactcaagccccagaatcccaggagagccaagaggagctccattatgccacgctcaacttcccaggcgtcagacccaggcctgaggcccggatgcccaagggcacccaggcggattatgcagaagtcaagttccaatgagggtctcttaggctttaggactgggacttcggctagggaggaaggtagagtaagaggttgaagataacagagtgcaaagtttccttctctccctctctctctctctttctctctctctctctctttctctctcttttaaaaaaacatctggccagggcacagtggctcacgcctgtaatcccagcactttgggaggttgaggtgggcagatcgcctgaggtcgggagttcgagaccagcctggccaacttggtgaaaccccatctctacaaaaaatacaaaacatagctgggcttggtggtgtgtgcctgtagtcccagctgtcagacatttaaaccagagcaactccatctggaataggagctgaataaaatgaggctgagacctactgggctgcattctcagacagtggaggcattctaagtcacaggatgagacaggaggtccgtacaagatacaggtcataaagactttgctgataaaacagattgcagtaaagaagccaaccaaatcccaccaaaaccaagttggccacgagagtgacctctggtcgtcctcactgctacactcctgacagcaccatgacagtttacaaatgccatggcaacatcaggaagttacccgatatgtcccaaaagggggaggaatgaataatccaccccttgtttagcaaataagcaagaaataaccataaaagtgggcaaccagcagctctaggcgctgctcttgtctatggagtagccattcttttgttcctttactttcttaataaacttgctttcaccttaaaaaaaaaaaaaaag

SEQ ID NO: 28 - protein, 697 aa, Artificial Sequence
  Description of Artificial Sequence Siglec-BMSL3-995-2
MLLPLLLSSLLGGSQAMDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSVQYPPENLRVMVSQANRTVLENLGNGTSLPVLEGQSLCLVCVTHSSPPARLSWTQRGQVLSPSQPSDPGVLELPRVQVEHEGEFTCHARHPLGSQHVSLSLSVHYSPKLLGPSCSWEAEGLHCSCSSQASPAPSLRWWLGEELLEGNSSQDSFEVTPSSAGPWANSSLSLHGGLSSGLRLRCEAWNVHGAQSGSILQLPDKKGLISTAFSNGAFLGIGITALLFLCLALIIMKILPKRRTQTETPRPRFSRHSTILDYINVVPTAGPLAQKRNQKATPNSPRTPLPPGAPSPESKKNQKKQYQLPSFPEPKSSTQAPESQESQEELHYATLNFPGVRPRPEARMPKGTQADYAEVKFQ

SEQ ID NO: 29 - DNA, 2529 bp, Artificial Sequence; identical to SEQ ID NO: 15
  Description of Artificial Sequence Siglec-BMSL3-hIg

SEQ ID NO: 30 - protein, 779 aa, Artificial Sequence [sequence omitted]

SEQ ID NO: 31 - DNA, 2052 bp, Artificial Sequence
  Description of Artificial Sequence Siglec-BMSL3a-hIg
ccacgcgtccgggccccagggctcagcttccgccttcggcttccccttctgccaagagccctgagccactcacagcacgaccagagaacaggcctgtctcaggcaggccctgcgcctcctatgcggagatgctactgccactgctgctgtcctcgctgctgggcgggtcccaggctatggatgggagattctggatacgagtgcaggagtcagtgatggtgccggagggcctgtgcatctctgtgccctgctctttctcctacccccgacaagactggacagggtctaccccagcttatggctactggttcaaagcagtgactgagacaaccaagggtgctcctgtggccacaaaccaccagagtcgagaggtggaaatgagcacccggggccgattccagctcactggggatcccgccaaggggaactgctccttggtgatcagagacgcgcagatgcaggatgagtcacagtacttctttcgggtggagagaggaagctatgtgagatataatttcatgaacgatgggttctttctaaaagtaacagtgctcagcttcacgcccagaccccaggaccacaacaccgacctcacctgccatgtggacttctccagaaagggtgtgagcgcacagaggaccgtccgactccgtgtggcctatgcccccagagaccttgttatcagcatttcacgtgacaacacgccagccctggagccccagccccagggaaatgtcccatacctggaagcccaaaaaggccagttcctgcggctcctctgtgctgctgacagccagccccctgccacactgagctgggtcctgcagaacagagtcctctcctcgtcccatccctggggccctagacccctggggctggagctgcccggggtgaaggctggggattcagggcgctacacctgccgagcggagaacaggcttggctcccagcagcgagccctggacctctctgtgcagtatcctccagagaacctgagagtgatggtttcccaagcaaacaggacagtcctggaaaaccttgggaacggcacgtctctcccagtactggagggccaaagcctgtgcctggtctgtgtcacacacagcagccccccagccaggctgagctggacccagaggggacaggttctgagcccctcccagccctcagaccccggggtcctggagctgcctcgggttcaagtggagcacgaaggagagttcacctgccacgctcggcacccactgggctcccagcacgtctctctcagcctctccgtgcactaggatccggagcccaaatcttgtgacaaaactcacacatgcccaccgtgcccagcacctgaattcgagggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagccccgctccccgggctctcgcggtcgcacgaggatgcttctaga

SEQ ID NO: 32 - protein, 619 aa, Artificial Sequence
  Description of Artificial Sequence Siglec-BMSL3a-hIg
MLLPLLLSSLLGGSQAMDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSVQYPPENLRVMVSQANRTVLENLGNGTSLPVLEGQSLCLVCVTHSSPPARLSWTQRGQVLSPSQPSDPGVLELPRVQVEHEGEFTCHARHPLGSQHVSLSLSVHDPEPKSCDKTHTCPPCPAPEFEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - DNA, 21 bp, Artificial Sequence
  Description of Artificial Sequence L3 forward primer
tgctcagcttcacgcccagac

SEQ ID NO: 34 - DNA, 20 bp, Artificial Sequence
  Description of Artificial Sequence L3 reverse primer
tgcacggagaggctgagaga

SEQ ID NO: 35 - DNA, 19 bp, Artificial Sequence
  Description of Artificial Sequence S1 forward primer
ctcagaagcctgatgtcta

SEQ ID NO: 36 - DNA, 18 bp, Artificial Sequence
  Description of Artificial Sequence S1 reverse primer
gagaagtgggaggtcgtt

SEQ ID NO: 37 - DNA, 18 bp, Artificial Sequence
  Description of Artificial Sequence S2 forward primer
ctgctgggcccctcctgc

SEQ ID NO: 38 - DNA, 19 bp, Artificial Sequence
  Description of Artificial Sequence S2 reverse primer
gacgttccaggcctcacag

SEQ ID NO: 39 - DNA, 20 bp, Artificial Sequence
  Description of Artificial Sequence P1 forward primer
ccttcggcttccccttctgc

SEQ ID NO: 40 - DNA, 19 bp, Artificial Sequence
  Description of Artificial Sequence P1 reverse primer
cgttggtttggttccttgg

SEQ ID NO: 41 - DNA, 19 bp, Artificial Sequence
  Description of Artificial Sequence P2 forward primer
cacactgagctgggtcctg

SEQ ID NO: 42 - DNA, 19 bp, Artificial Sequence
  Description of Artificial Sequence P2 reverse primer
gacgttccaggcctcacag

SEQ ID NO: 43 - DNA, 19 bp, Artificial Sequence
  Description of Artificial Sequence P3 forward primer
cacactgagctgggtcctg

SEQ ID NO: 44 - DNA, 20 bp, Artificial Sequence
  Description of Artificial Sequence P3 reverse primer
gaaaagaagagccgtgatgc

SEQ ID NO: 45 - DNA, 18 bp, Artificial Sequence
  Description of Artificial Sequence L3-TM forward primer
tgcagctgccagataaga

SEQ ID NO: 46 - DNA, 18 bp, Artificial Sequence
  Description of Artificial Sequence L3-TM reverse primer
ggcttgagtggatgattt

SEQ ID NO: 47 - DNA, 19 bp, Artificial Sequence
  Description of Artificial Sequence S1 forward primer
ctccgaagcctgatgtcta

SEQ ID NO: 48 - DNA, 18 bp, Artificial Sequence
  Description of Artificial Sequence S1 reverse primer
gagaagtgggaggtcgtt

SEQ ID NO: 49 - DNA, 18 bp, Artificial Sequence
  Description of Artificial Sequence S2 forward primer
ctgctgggcccctcctgc

SEQ ID NO: 50 - DNA, 19 bp, Artificial Sequence
  Description of Artificial Sequence S2 reverse primer
gacgttccaggcctcacag

SEQ ID NO: 51 - DNA, 20 bp, Artificial Sequence
  Description of Artificial Sequence Beta-actin forward primer
gtggggcgccccaggcacca

SEQ ID NO: 52 - DNA, 23 bp, Artificial Sequence
  Description of Artificial Sequence Beta-actin reverse primer
ctccttaatgtcacgcacgattc

SEQ ID NO: 53 - DNA, 18 bp, Artificial Sequence
  Description of Artificial Sequence L3-TM forward primer
tgcagctgccagataaga

SEQ ID NO: 54 - DNA, 18 bp, Artificial Sequence
  Description of Artificial Sequence L3-TM reverse primer
ggcttgagtggatgattt

SEQ ID NO: 55 - DNA, 36 bp, Artificial Sequence
  Description of Artificial Sequence GST-SiglecBMSL3cyto (wt) forward primer
gcggccaggaattccaagagacggactcagacagaa

SEQ ID NO: 56 - DNA, 33 bp, Artificial Sequence
  Description of Artificial Sequence GST-SiglecBMSL3cyto (wt) reverse primer
gcggccctcgagtcattggaacttgacttctgc

SEQ ID NO: 57 - DNA, 57 bp, Artificial Sequence
  Description of Artificial Sequence GST-SiglecBMSL3Y641F forward primer
ccagaatcaaagaagaaccagaaaaagcagtttcagttgcccagtttcccagaaccc

SEQ ID NO: 58 - DNA, 56 bp, Artificial Sequence
  Description of Artificial Sequence GST-SiglecBMSL3Y641F reverse primer
gggttctgggaaactgggcaactgaactgctttttctggttcttctttgattctgg

SEQ ID NO: 59 - DNA, 45 bp, Artificial Sequence
  Description of Artificial Sequence GST-SiglecBMSL3Y667F forward primer
gagagccaagaggagctccattttgccacgctcaacttcccaggc

SEQ ID NO: 60 - DNA, 45 bp, Artificial Sequence
  Description of Artificial Sequence GST-SiglecBMSL3Y667F reverse primer
gcctgggaagttgagcgtggcaaaatggagctcctcttggctctc

SEQ ID NO: 61 - DNA, 51 bp, Artificial Sequence
  Description of Artificial Sequence GST-SiglecBMSL3Y691F forward primer
gcggccctcgagtcattggaacttgacttctgcaaaatccgcctgggtgcc

SEQ ID NO: 62 - DNA, 51 bp, Artificial Sequence; identical to SEQ ID NO: 61
  Description of Artificial Sequence GST-SiglecBMSL3Y691F reverse primer

SEQ ID NO: 63 - DNA, 39 bp, Artificial Sequence
  Description of Artificial Sequence GST-SiglecBMSL3Y641 alone forward primer
gcggccaggaattccatcaatgtggtcccgacggctggc

SEQ ID NO: 64 - DNA, 36 bp, Artificial Sequence
  Description of Artificial Sequence GST-SiglecBMSL3Y641 alone reverse primer
gcggccctcgagtcaatggagctcctcttggctctc

SEQ ID NO: 65 - DNA, 109 bp, Artificial Sequence
  Description of Artificial Sequence SiglecBMSL3a hIg forward primer
ccgcctaagctttccccttctgccaagagccctgagccctgagccactcacagcacgaccagagaacaggcctgtctcaggcaggccctgcgcctcctatgcggagatg

SEQ ID NO: 66 - DNA, 35 bp, Artificial Sequence
  Description of Artificial Sequence SiglecBMSL3a hIg reverse primer
gaagatctgaaccatggttatagtgcacggagagg

SEQ ID NO: 67 - DNA, 109 bp, Artificial Sequence; identical to SEQ ID NO: 65
  Description of Artificial Sequence SiglecBMSL3 hIg forward primer

SEQ ID NO: 68 - DNA, 35 bp, Artificial Sequence
  Description of Artificial Sequence SiglecBMSL3 hIg reverse primer
gaagatctgaaccatggttaggagaatgccgttga

SEQ ID NO: 69 - DNA, 241 bp, Artificial Sequence
  Description of Artificial Sequence partial 5′ sequence of 3421048
caggcctgtctcaggcaggccctgcgcctcctatgcggagatgctactgccactgctgctgtcctcgctgctgggcgggtcccangctatggatgggagattctggatacgagtgcaggagtcagtgatggtgccggagggcctgtgcatctctgtgccctgctctttctcctacccccgacaggactggacagggtctaccccagcttatggctactggttcaaagcagtgactgagaca

SEQ ID NO: 70 - DNA, 21 bp, Artificial Sequence
  Description of Artificial Sequence Siglec 10 L3 probe
tgctcagcttcacgcccagac

SEQ ID NO: 71 - DNA, 20 bp, Artificial Sequence
  Description of Artificial Sequence Siglec 10 L3 probe
tgcacggagaggctgagaga

(19) United States
(12) Patent Application Publication
(10) Pub. No.: US 2003/0036631 A1
(43) Pub. Date: Feb. 20, 2003

(54) Novel siglecs and uses thereof

(76) Inventors: Malinda Longphre, Oakland, CA (US); Han Chang, Princeton Junction, NJ (US); Gena Whitney, Lawrenceville, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 09/910,600

(22) Filed: Jul. 20, 2001

Related U.S. Application Data
(60) Provisional application No. 60/220,139, filed on Jul. 21, 2000.

(51) Int. Cl. C07K 14/705

(52) U.S. Cl. 530/350; 530/388.1

(57) ABSTRACT

The present invention provides proteins, peptide fragments, nucleic acid molecules and fragments thereof, complementary sequences, allelic forms, homologues, antibodies and variants of SIGLEC-BMS sequences, which are new members of the Sialoadhesin subgroup having protein homology to CD33, and methods of using these molecules.

This application is based on a provisional application, U.S. Serial No. 60/220,139, filed Jul. 21, 2000, the contents of which are hereby incorporated by reference in their entirety into this application.

Throughout this application, various publications are referenced. The disclosures of these publications are hereby incorporated by reference herein in their entireties.

### FIELD OF THE INVENTION

The present invention relates to sialoadhesin nucleotide sequences, herein designated “SIGLEC-BMS”, and novel SIGLEC polypeptides, and uses thereof.

### BACKGROUND OF THE INVENTION

A group of sialic acid-dependent adhesion molecules has been described within the superfamily of immunoglobulin-like molecules (Kelm, S. et al., 1998 Eur. J. Biochem 255:663-672). The term “Siglec” has been adopted to describe this family (Sialic acid-binding Ig-related lectins). To date, the members of the group include Siglec-1 (sialoadhesin), Siglec-2 (CD22), Siglec-3 (CD33), Siglec-4 (myelin-associated glycoprotein or MAG), Siglec-4b (Schwann cell myelin protein or SMP), Siglec-5 (OB-BP2), Siglec-6 (OB-BP1, CD33L), Siglec-7, and Siglec-8 (Table 1).

The biological activity of the protein members of the Siglec group is, for the most part, not understood. However, Siglec proteins are thought to be involved in diverse biological processes such as hemopoiesis, neuronal development and immunity (Vinson, M. et al., 1996 supra). Studies also suggest that these proteins mediate cell adhesion/cell signaling through recognition of sialyated cell surface glycans (Kelm, S. et al., 1996 Glycoconj. J. 13:913-926; Kelm, S. et al., 1998 Eur. J. Biochem. 255:663-672; Vinson, M. et al., 1996 J. Biol. Chem. 271:9267-9272).

TABLE 1
Members of the Siglec protein family and their distribution

| Protein: | Expressed on: | Recognizes: | Target Cell | References: |
|---|---|---|---|---|
| Siglec-1 | Macrophage | Siaα2,3 Galβ1,3 GalNAc | Myeloid cells | Crocker et al. 1194 EMBO J. 13: 4490-4503 |
| (Sialoadhesin) | Subpopulations | Siaα2,3 Galβ1,3/4 GlcNAc | Neutrophils | Crocker et al. 1997 Glycoconjugate J. 14: 601-609 |
| | | | | Vinson et al. 1996 J. Biol. Chem. 271: 9267-9272 |
| | | | | Kelm et al. 1998 Eur. J. Biochem. 255: 663-672 |
| | | | | Barnes et al. 1999 Blood 93: 1245-1252 |
| Siglec-2 | B-cells | Siaα2,6 Galβ1,4 GlcNAc | Lymphocytes | Kelm et al. 1998 Eur. J. Biochem. 255: 663-672 |
| (CD22) | | | | |
| Siglec-3 | Myelomonocytic cells | Siaα2,3 Galβ1,3 GalNAc | Myelomonocytic | Freeman et al. 1995 Blood 85: 2005-2012 |
| (CD33) | | Siaα2,3 Galβ1,3/4 GlcNAc | cells | Kelm et al. 1998 Eur. J. Biochem. 255: 663-672 |
| | | | | Taylor et al. 1999 J. Biol. Chem. 274 11505-11512 |
| Siglec-4a | Oligodendricytes | Siaα2,3 Galβ1,3 GalNAc | Neurons | Kelm et al. Eur. J. Biochem. 255: 663-672 |
| | Schwann cells | | Oligodendricytes | |
| Siglec-4b | Schwann cells (quail) | Siaα2,3 Galβ1,3 GalNAc | Neurons | Kelm et al. 1998 Eur. J. Biochem. 255: 663-672 |
| Siglec-5 | Neutrophils | Siaα2,3 Galβ1,3 GalNAc | Erythrocytes | Cornish et al. 1998 Blood 92: 2123-2132 |
| (OB-BP2) | Monocytes | Siaα2,6 Galβ1,3 GalNAc | | |
| Siglec-6 | Placenta | Not known | Not known | Takei et al. 1997 Cytogen. Cell. Genet. 78: 295-300 |
| (OB-BP1, CD33L) | B-cells | | | Patel et al. 1999 J. Biol. Chem. 274: 22729-22738 |
| Siglec-7 | Granulocytes, monocytes, | Siaα2,3 Galβ1,3 GalNAc | Not known | Nicoll et al. 1999 J. Biol. Chem. 274: 34089-34095 |
| (AF170485) | NK cells, CD8 + T cells | Siaα2,6 Galβ1,3 GalNAc | | |
| | Eosinophils | | | |
| (L3a-604, L3b-595, | | | | |
| L3c-995, L3d963) | | | | |
| Siglec-8 | Eosinophils | NAcα2,3 Galβ1,4 Glc | Not known | Floyd et al. 2000 J. Biol. Chem. 275: 861-866 |
| (AF 195092) | | | | |

The known Siglec proteins are expressed in diverse hemopoietic cell types, yet they all share a similar structure including a single N-terminal V-set domain (membrane-distal) followed by variable numbers of extracellular C2-set domains, a transmembrane domain, and a short cytoplasmic tail (FIG. 1). Additionally, the terminal V-set domain has an unusual intrasheet disulfide bridge that is unique among members of the Ig superfamily (Williams, A. F. and Barclay, A. N. 1988 Annu. Rev. Immunol. 6:381-405; Williams, A. F., et al., 1989 Cold Spring Harbor Symp. Quant. Biol 54:637-647; Pedraza, L., et al., 1990 J. Cell. Biol. 111:2651-2661).

Results of various research approaches, including truncating mutants (Nath, D., et al., J. Biol. Chem. 270:26184-26191), site-directed mutagenesis (Vinson, M., et al., 1996 J. Biol. Chem. 271:9267-9272; Van der Merwe, P. a., et al., 1996 J. Biol Chem. 271:9273-9280), X-ray crystallography and NMR (discussed in: Crocker, P. R., et al., 1997 Glycoconjugate J. 14:601-609) have unequivocally demonstrated that the GFCC′C″ face of the N-terminal V-set domain of known Siglec proteins interact with sialic acid. Thus, the V-set domain mediates cell-to-cell adhesion by interacting with sialic acid. In particular, an arginine residue within the V-set domain is a key amino acid residue for binding to sialic acid (Vinson, M., et al., 1996 supra).

The purported ligands for the known Siglec proteins are glycoproteins or glycolipids on other cells, or in some instances on the same cell, modified to include sugars or sialic acid (Table 1). There are approximately 40 naturally occurring sialic acids (Sia) adding to the structural diversity of cell surface glycoproteins. The most common are NeuSAc, Neu9Ac2 and Neu5Gc, occurring in terminal positions linked to other sugars like Gal, GalNAc, GlcNAc and Sia itself on glycoproteins and glycolipids. It is postulated that the pattern of expression of sialic acids in certain cell types is controlled by specific expression of sialyltransferases (Paulson, J. C. et al., 1989 J. Biol. Chem. 264:10931-10934). The Siglec proteins may recognize not only the terminal sialic acids but also the context of these moieties based on pre-terminal sugars to which they are attached (Kelm, S., et al., 1996 Glycoconj. J. 13:913-926).

Siglecs may mediate cell to cell adhesion by functioning as sialic acid-dependent lectins with distinct specificities for the type of sialic acid and its linkage to subterminal sugars (Kelm, S., 1994 supra; Powell, L. D., et al., 1994 J. Biol. Chem. 269:10628-10636; Sjoberg, E., et al., 1994 J. Cell Biol. 126:549-562; Collins, B., E., et al., 1997 J. Biol. Chem. 272:1248-1255). For example, cells expressing Siglec-1 recognize the sequences Neu5Acα2,3Galβ1,3GalNAc and Neu5Acα2,3Galβ1,3(4)GlcNAc on glycoproteins and glycolipids (Kelm, S., et al., 1994 Curr. Biol. 4:965:72; Crocker, P. R., et al., 1991 EMBO J. 10:1661).

Siglecs are also postulated to be involved in cis-interaction in which a Siglec protein recognizes glycoconjugates on the same cell. Such cis-interaction may regulate intercellular adhesion for CD22 (Braesch-Andersen, S. and Stamenkovic, I. 1994 J. Biol. Chem. 269:11783-11786; Hanasaki, K., et al., 1995 J. Biol. Chem. 270:7533-7542), CD33 (Freeman, S. D., et al., 1995 supra), and MAG (as discussed in Freeman, S. D., et al., 1995 supra).

The amino acid sequences of the cytoplasmic tails of several Siglec proteins strongly suggest that they participate in intracellular signaling. For example, Siglec-2 has 6 tyrosines in the cytoplasmic domain, two of which reside within ITAM (Immunotyrosine-based activation motifs) motifs which mediate activation, and four within ITIM (Immunotyrosine-based inhibition motifs) motifs which mediate inhibition (Taylor, V. et al., 1999 J. Biol. Chem. 274:11505-11512). Phosphorylation of the ITAM motif tyrosines would allow recruitment of Src, whereas phosphorylation of ITIM motif tyrosines would allow recruitment of SHP-1 and SHP-2. Siglec-3 contains two ITIMs that recruit SHP-1 and SHP-2 upon phosphorylation (Taylor, V. et al., 1999 supra). Siglec-6 also has putative SLAM-like signaling motifs in the cytoplasmic tail; SLAM is an acronym for Signaling Lymphocyte Activation Molecule. (Patel, N. et al., 1999 J. Biol. Chem. 274:22729-22738).

Other biological activities of Siglecs have been postulated. There is mounting evidence that inflammatory cell infiltrates play a significant role in driving the pathogenesis of asthma and other allergic diseases by damaging tissue and releasing pro-inflammatory agents. Activated eosinophils, neutrophils, macrophages, mast cells and lymphocytes increase in number at sites of inflammation and each are capable of modifying the overall inflammatory response (Busse, W. W. 1998 J. Allergy Clin. Immunol. 102:S17-22). Eosinophils are of particular interest in asthma and allergy due to their conspicuous appearance at the sites of allergen-driven inflammation (Kroegel, C. et al., 1994 Eur. Respir. J. 7:519-543; Haczku, A. 1998 Acta. Microbiol. Immunol. Hung. 45:19-29; Boyce, J. A. 1997 Allergy Asthma Proc. 18:293-300). Through release of toxic granule proteins, pro-inflammatory lipid mediators and cytokines, eosinophils have been implicated as major players in airway remodeling and hyperresponsiveness in asthma (Durham, S. R. 1998 Clin. Exp. Allergy 28 Suppl. 2:11-6).

CD33 (e.g., Siglec 3) is considered to be a member of the Siglec family based on its structural similarity with other Siglecs and its ability to bind to sialic acid. CD33 (Siglec-3; 67 kDa, Human sequence in EMBL/GENBANK M23197, Mouse sequence in EMBL/GENBANK S71345/S71403) was originally isolated from human myeloid cells (Andrews, R. G. et al 1983 Blood 62:124; Griffin, J. D. et al 1984 Leuk Res. 8:521; Peiper, S. C. 1988 Blood 72:314-321; Peirelli, L et al., 1993 Br. J. Haematol. 84:24;). Additional CD33 homologues have been identified, including SAF-2 (European patent #EP 0 924 297 A1) and SAF-4 (published patent application No. WO9853840) and AF135027 (Genbank).

The sequences of the human (Simmons, D. L. and Seed, B. 1988 J. Immunol. 141: 2797) and murine (Tchilian, E. Z., et al., 1994 Blood 83:3188) cDNA clones predict that CD33 encodes a Siglec having only two C-set domains, making it the smallest of known Siglecs. CD33 binds to NeuAcα2,3Galβ1,3GalNAc in O-glycans and NeuAcα2,3Falβ1,3(4)GlcNAc in N-glycans (Freeman, S. et al., 1995 Blood 85:2005-2012). Cells expressing CD33 must be desialylated in order to bind to cells bearing the appropriate sialoglycoconjugate, suggesting that inhibitory cis-interactions may regulate or block any adhesion function (Freeman, S. et al., 1995 supra). Additionally, CD33 has the conserved arginine residue in the V-set domain.

CD33 is a clinically important diagnostic marker for distinguishing myeloid from lymphoid leukemia (Griffin, J. D., et al., Leuk. Res. 8:521; Matutes, E. et al., 1985 Haematol. Oncol. 3:179; Bain, B. J. ed 1990 in: Leukaemia Diagnosis. A Guide to the FAB Classification, pp 61, London, UK, Gower Medical). CD33 expression has been associated with myelomonocytic progenitors, monocytes and macrophages, suggesting that it plays a role in regulating myeloid cell differentiation (Peiper, S. C., et al., supra; Peirelli, L. et a., supra; Andrews, R. G., et al., supra; Griffin, J. D., et al., supra; Nakamura, Y., et al., 1994 Blood 83:1442; Bernstein, I. D., et al., 1987 J. Clin. Invest. 79:1153).

Sequences that are predicted to encode SIGLEC proteins that are structurally similar to CD33 have been previously isolated and characterized. For example, sequences that are similar to CD33 include: CD33L1 and CD33L2 which are postulated to be related as a result of differential-splicing and were isolated from a human placental cDNA library (Takei, Y., et al., 1997 Cytogent. Cell. Genet. 78:295-300); Siglec-5 (Cornish, A. L., et al., 1998 Blood 92:2123-2132) which was isolated from a human activated monocyte library (EST library #pHMQCD14) and is postulated to be expressed from the same gene that expresses OB-BP2 (Genbank Accession #: M23197) which is a leptin-binding protein, as they have nearly identical sequences; and Siglec-6 (OB-BP1) which was isolated from the TF-1 human erythroleukemic cell line (Patel, N., et al., 1999 J. Biol. Chem. 274:22729-22738). Sequences for Siglecs 7 and 8 have also recently been described (Nicoll, G., et al., 1999 J. Biol. Chem. 274:34089-34095; Floyd, H., et al., 2000 J. Biol. Chem. 275:861-866).

The present invention relates to the discovery of nucleotide sequences (e.g., Siglec-BMS-L3a, -L3b, -L3c, -L3d, -L4a, -L5a, and -L5b) and novel Siglec proteins encoded by them having structural homology to CD33/Siglec 3.

### SUMMARY OF THE INVENTION

The invention provides isolated nucleic acid molecules encoding the SIGLEC-BMS proteins of the invention, and methods for uses thereof. For example, the nucleotide sequences of the invention include: Siglec-BMS-L3a, -L3b, -L3c, -L3d, -L4a, -L5a, L5b, and -L3-995-2 as shown in FIGS. 2A, 3A, 4A, 5A, 7A, 8A, 9A, and 6A respectively.

The invention further provides SIGLEC-BMS protein molecules. Specific embodiments of SIGLEC-BMS proteins of the invention include: SIGLEC-BMS-L3a, -L3b, -L3c, -L3d, -L4a, -L5a, -L5b, and -L3-995-2 as shown in FIGS. 2B, 3B, 4B, 5B, 7B, 8B, 9B, and 6B respectively.

The nucleic acid molecules of the invention include portions of the Siglec-BMS sequences, such as oligonucleotides, or fragments thereof. The nucleic acid molecules of the invention also include peptide nucleic acids (PNA), and antisense molecules that react with the nucleic acid molecules of the invention.

The present invention also encompasses various nucleotide sequences that represent different forms of the Siglec-BMS genes and transcripts, such as different allelic forms, polymorphic forms, alternative precursor transcripts, mature transcripts, and differentially-spliced transcripts. Additionally, recombinant nucleic acid molecules that are codon usage variants of the Siglec-BMS sequences are provided.

The present invention includes the polynucleotides encoding Siglec-BMS in recombinant expression vectors and host-vector systems that include the expression vectors. One embodiment provides various host cells introduced with recombinant vectors that include the Siglec-BMS sequences of the invention.

The present invention provides methods for using isolated and substantially purified Siglec-BMS nucleotide sequences as nucleic acid probes and primers, for using SIGLEC-BMS polypeptides as antigens for the production of anti-SIGLEC-BMS antibodies, and for using SIGLEC-BMS polypeptides for obtaining and detecting SIGLEC-BMS ligands. The Siglec-BMS probes and primers, and the anti-SIGLEC-BMS antibodies are useful in diagnostic assays and kits for the detection of naturally occurring Siglec-BMS nucleotide sequences and SIGLEC-BMS protein sequences present in biological samples.

The invention also relates to antisense molecules capable of reacting with the Siglec-BMS nucleotide sequences of the invention, thereby disrupting expression of genomic sequences.

The invention also relates to therapeutic agents including agonists, antibodies, antagonists or inhibitors of the activity of SIGLEC-BMS proteins. These compositions are useful for the prevention or treatment of conditions associated with the presence or the deficiency of SIGLEC-BMS proteins.

The present invention further provides pharmaceutical compositions for treating immune system diseases, such as asthma, leukemia, or other allergic or inflammatory diseases, comprising at least one SIGLEC-BMS protein and a pharmaceutically acceptable carrier. The present invention further provides pharmaceutical compositions comprising an antibody or antibody fragment thereof, that recognizes at least one SIGLEC-BMS protein, in an acceptable carrier.

Kits comprising pharmaceutical compositions therapeutic for immune system diseases are also encompassed by the invention. In one embodiment, a kit comprising one or more of the pharmaceutical compositions of the invention is used to treat an immune system disease, e.g. asthma, leukemia, or other allergic or inflammatory diseases.

### BRIEF DESCRIPTION OF THE FIGURES

FIG. 1: Schematic representation of the predicted structures of the SIGLEC family of proteins.

FIG. 2: A) The nucleotide sequence of Siglec-BMS-L3a (SEQ ID NO.:1); B) the predicted amino acid sequence of SIGLEC-BMS-L3a (SEQ ID NO.:8), as described in Example 1, infra.

FIG. 3: A) The nucleotide sequence of Siglec-BMS-L3b (SEQ ID NO.:2); B) the predicted amino acid sequence of SIGLEC-BMS-L3b (SEQ ID NO.:9), as described in Example 1, infra.

FIG. 4: A) The nucleotide sequence of Siglec-BMS-L3c (SEQ ID NO.:3); B) the predicted amino acid sequence of SIGLEC-BMS-L3c (SEQ ID NO.:10), as described in Example 1, infra.

FIG. 5: A) The nucleotide sequence of Siglec-BMS-L3d (SEQ ID NO.:4); B) the predicted amino acid sequence of SIGLEC-BMS-L3d (SEQ ID NO.: 11), as described in Example 1, infra.

FIG. 6: A) The nucleotide sequence of Siglec-BMS-L3-995-2 (SEQ ID NO.:27), the ATG start codon and the splicing locations are shaded, the open boxed region represents the transmembrane domain; B) the predicted amino acid sequence of SIGLEC-BMS-L3-995-2 (SEQ ID NO.:28), as described in Example 14, infra.

FIG. 7: A) The nucleotide sequence of Siglec-BMS-L4a (SEQ ID NO.:5); B) the predicted amino acid sequence of SIGLEC-BMS-L4a (SEQ ID NO.:12), as described in Example 1, infra.

FIG. 8: A) The nucleotide sequence of Siglec-BMS-L5a (SEQ ID NO.:6); B) the predicted amino acid sequence of SIGLEC-BMS-L5a (SEQ ID NO.:13), as described in Example 1, infra.

FIG. 9: A) The nucleotide sequence of Siglec-BMS-L5b (SEQ ID NO.:7); B) the predicted amino acid sequence of SIGLEC-BMS-L5b (SEQ ID NO.:14), as described in Example 1, infra.

FIG. 10: A) A Northern blot analysis showing the distribution of Siglec-BMS-L3 transcripts in human tissue; B) a schematic map showing the location of the probe sequences, as described in Example 2, infra.

FIG. 11: A) A table showing the results of an RT-PCR analysis showing the distribution of Siglec-BMS-L3 transcripts in human tissue; B) a schematic map showing the location of the primers/PCR products, as described in Example 3, infra.

FIG. 12: A) Histograms showing the distribution of Siglec-BMS-L3 transcripts in human tissue and cell lines, as detected by quantitative RT-PCR analysis; B) a quantitative RT-PCR analysis showing expression levels of Siglec-BMS-L3 transcripts in purified human white blood cells from two individual human subjects; C) a schematic map showing the location of the primers/PCR products, as described in Example 4, infra.

FIG. 13: A graph showing the results of a binding assay in which immobilized SIGLEC-BMSL3-hIg fusion protein (e.g., extracellular domain of SIGLEC-BMS-L3) binds to various blood cell populations or cell lines, as described in Example 8, infra.

FIG. 14: A graph showing the results of a binding assay in which COS7 cells, expressing full-length SIGLEC-BMS-L3 protein, bind to various blood cell populations or cell lines, as described in Example 9, infra.

FIG. 15: A schematic representation of the various GST fusion proteins comprising the cytoplasmic tail of wild-type and mutated SIGLEC-BMS-L3 protein, including L3cyto-wt, L3cyto-Y641F, L3cyto-Y667F, L3cyto-Y691F, and L3cyto-Y641 alone. Also depicted are hIg (human immunoglobulin) fusion proteins comprising the non-spliced (995-2, SIGLEC-BMSL3 hIg) and spliced (526604, SIGLEC-BMSL3a hIg) extracellular domains of SIGLEC-BMSL3 protein, as described in Example 10, infra.

FIG. 16: Graphs showing the results of kinase assays involving various substrates including GST fusion proteins comprising the cytoplasmic tail of wild-type and mutated SIGLEC-BMS-L3 protein reacted with various tyrosine kinases, as described in Example 12, infra: A) lck kinase; B) ZAP70 kinase; C) emt kinase; and D) JAK3 kinase. E) A graph showing the results of kinase assays involving a GST fusion protein substrate, comprising the cytoplasmic tail of wild-type SIGLEC-BMS-L3 protein, and various tyrosine kinases including lck, ZAP70, emt, and JAK3. F) A graph showing the results of kinase assays involving LAT substrate and various tyrosine kinases including lck, ZAP70, emt, and JAK3. G) A graph showing results of tyrosine phosphorylation of GST fusion proteins comprising the cytoplasmic tail of wild-type and various Y→F mutants with a tyrosine kinase mix.

FIG. 17: A) Results of immunoprecipitation experiments demonstrating that SHP-1 and SHP-2 associate with the phosphorylated SIGLEC-BMSL3 cytoplasmic tail, as described in Example 13, infra. B) Depicts binding of SHP-1 and SHP-2 to Y667 ITIM by ELISA, as described in Example 13.

FIG. 18: Depicts the nucleotide and amino acid sequences of the cytoplasmic tail domain of Siglec-BMS-L3a fused to a GST protein as described in Example 10, infra. A) The nucleotide sequence of L3cyto-wt (SEQ ID NO: 17); B) the amino acid sequence of L3cyto-wt (SEQ ID NO:22).

FIG. 19: Depicts the nucleotide and amino acid sequences of the cytoplasmic tail domain of Siglec-BMS-L3a fused to a GST protein, as described in Example 10, infra. A) The nucleotide sequence of L3cyto-Y641F (SEQ ID NO:18); B) the amino acid sequence of L3cyto-Y641F (SEQ ID NO:23).

FIG. 20: Depicts the nucleotide and amino acid sequences of the cytoplasmic tail domain of Siglec-BMS-L3a fused to a GST protein, as described in Example 10, infra. A) The nucleotide sequence of L3cyto-Y667F (SEQ ID NO:19); B) the amino acid sequence of L3cyto-Y667F (SEQ ID NO:24).

FIG. 21: Depicts the nucleotide and amino acid sequences of the cytoplasmic tail domain of Siglec-BMS-L3a fused to a GST protein, as described in Example 10, infra. A) The nucleotide sequence of L3cyto-Y691F (SEQ ID NO:20); B) the amino acid sequence of L3cyto-Y691F (SEQ ID NO:25).

FIG. 22: Depicts the nucleotide and amino acid sequences of the cytoplasmic tail domain of Siglec-BMS-L3a fused to a GST protein, as described in Example 10, infra. A) The nucleotide sequence of L3cyto-Y641 alone (SEQ ID NO:21); B) the amino acid sequence of L3cyto-Y641 alone (SEQ ID NO:26).

FIG. 23: Depicts the nucleotide and amino acid sequences of the extracellular domain of Siglec-BMS-L3a fused to a human immunoglobulin protein (hIg), as described in Example 11, infra. A) The nucleotide sequence of Siglec-BMS-L3a hIg (SEQ ID NO:31); B) the amino acid sequence of SIGLEC-BMS-L3a hIg (SEQ ID NO:32).

FIG. 24: Depicts the nucleotide and amino acid sequence of the extracellular domain of Siglec-BMS-L3 fused to a human immunoglobulin protein, as described in Example 11, infra. A) The nucleotide sequence of Siglec-BMS-L3 hIg (SEQ ID NO:29), B) the amino acid sequence of SIGLEC-BMS-L3 hIg (SEQ ID NO:30).

FIG. 25: Depicts the 697 amino acid sequence for Siglec-10, predicted based on the longest open reading frame (SEQ ID NO:15). The two spliced regions are indicated in gray, the cryptic splice acceptor site is underlined, the transmembrane domain is bolded and amino acids in the ITEM motifs in the cytoplasmic domain are boxed. The intron/exon boundaries are indicated with arrow and the domain numbers reflect the five Ig-like domains.

FIG. 26: Depicts the binding of polyacrylamide glycoconjugate to Siglec-10 as described in Example 15. Results shown are a mean +/−SD of 2 experiments, n=4-6 treatment/experiment.

FIG. 27: Depicts the Western blot of cell lysate probed with anti-Siglec-10 monoclonal antibody as described in Example 16.

FIG. 28: Depicts the results of in situ hybridization (ISH) detailing the distribution of Siglec-10 positive hybridization signals in non-human primate and human tissues as described in Example 17.

A) NHP spleen (Panels A, C, E); human spleen (Panels B, D, E)

B) NHP jejunum (Panels A, C, E); human liver (Panels B, D, E)

C) NHP Colon (Panels A-G)

D) NHP lymph nodes (Panels A, C, E); human lymph node (Panels B, D, E)

E) Human asthma lung (Panels A, C, E)

F) NHP lung (Panels A, B, D, E, G, H); human lung (Panels C, F, I)

### DETAILED DESCRIPTION OF THE INVENTION

In order that the invention herein described may be more fully understood, the following description is set forth.

The term “Siglec-BMS” as used herein refers to a protein family of sialic acid-binding Ig-like lectins sharing structural similarity including at least one Ig-like domain, a transmembrane domain, and a cytoplasmic tail. Typically, the Ig-like domain is extracellular and comprises an Ig(V) domain and an Ig(C) domain. Examples of SIGLEC-BMS proteins include, but are not limited to, L3a, L3b, L3c, L3d, L3-995-2, L4a, L5a, and L5b.

The term “Siglec-10” as used herein refers to a protein family of sialic acid -binding Ig-like lectins that shares structural similarity to CD33-related Siglecs, including multiple Ig-like domains, a transmembrane domain, and a cytoplasmic tail containing two ITIM-signaling motifs. The full length Siglec-10 protein comprises five Ig-like domains (Ig-DI, Ig-D2, Ig-D3, Ig-D4, and Ig-D5), and is designated SIGLEC-BMS-L3 in this application. The full length Siglec-10 protein is also termed as SIGLEC-BMS-L3-995-2. The terms Siglec-10, SIGLEC-BMS-L3, and SIGLEC-BMS-L3-995-2 are used interchangeably in this application.

The term “isolated” as used herein means a specific nucleic acid or polypeptide, or a fragment thereof, in which contaminants (i.e. substances that differ from the specific nucleic acid or polypeptide molecule) have been separated from the specific nucleic acid or polypeptide.

The term “purified” as used herein means a specific isolated nucleic acid or polypeptide, or a fragment thereof, in which substantially all contaminants (i.e. substances that differ from the specific nucleic acid or polypeptide molecule) have been separated from the specific nucleic acid or polypeptide.

As used herein, a first nucleotide or amino acid sequence is said to have sequence “identity” to a second reference nucleotide or amino acid sequence, respectively, when a comparison of the first and the reference sequences shows that they are exactly alike.

As used herein, a first nucleotide or amino acid sequence is said to be “similar” to a second reference sequence when a comparison of the two sequences shows that they have few sequence differences (i.e., the first and second sequences are nearly identical). For example, two sequences are considered to be similar to each other when the percentage of nucleotides or amino acids that differ between the two sequences may be between about 60% to 99.99%.

The term “complementary” as used herein refers to nucleic acid molecules having purine and pyrimidine nucleotides which have the capacity to associate through hydrogen bonding to form double stranded nucleic acid molecules. The following base pairs are related by complementarity: guanine and cytosine; adenine and thymine; and adenine and uracil. Complementary applies to all base pairs comprising two single-stranded nucleic acid molecules, or to all base pairs comprising a single-stranded nucleic acid molecule folded upon itself.

The term “fragment” of a SIGLEC-BMS-encoding nucleic acid molecule refers to a portion of a nucleotide sequence which encodes a polypeptide having the biological activity of a SIGLEC-BMS protein. A fragment of a Siglec-BMS molecule, is therefore, a nucleotide sequence having fewer nucleotides than the nucleotide sequence encoding the entire amino acid sequence of a SIGLEC-BMS protein, and which encodes a peptide having the biological activity of a SIGLEC-BMS protein

The term “fragment” of a SIGLEC-BMS polypeptide molecule refers to a portion of a polypeptide having the biological activity of a SIGLEC-BMS polypeptide.

The term “biological activity” of a SIGELC-BMS protein as used herein means that the protein functions as a cell adhesion molecule and/or the protein elicits the generation of an anti-SIGLEC-BMS antibody, where the SIGLEC-BMS protein binds with an anti-SIGLEC-BMS antibody.

The term “heterologous” as used herein refers to a non-SIGLEC-BMS protein or a fragment thereof. The heterologous molecule is fused (e.g., linked or joined) to a SIGLEC-BMS protein to facilitate isolation and/or purification of expressed SIGLEC-BMS gene product. Examples of heterologous molecules include, but are not limited to, human immunoglobulin constant region, a His-tag sequence, or a glutathione S-transferase (GST) sequence.

Molecules of the Invention

In its various aspects, as described in detail below, the present invention provides proteins, antibodies, nucleic acid molecules, recombinant DNA molecules, transformed host cells, generation methods, assays, therapeutic plus diagnostic methods and pharmaceutical, therapeutic or diagnostic compositions, all involving a Siglec-BMS protein or nucleic acids encoding them.

For the sake of convenience, the nucleotide sequences of Siglec-BMS (e.g., -L3a, -L3b, -L3c, -L3d, -L3-995-2,-L4a, -L5a, and -L5b) will be collectively referred to as “Siglec-BMS” or “Siglec nucleotide sequences of the invention”. Additionally, the proteins encoded by the Siglec-BMS nucleotide sequences include “SIGLEC-BMS-L3a, -L3b, -L3c, -L3d, -L3-995-2,-L4a, -L5a, and -L5b proteins” and collectively referred to as “SIGLEC-BMS proteins” or “SIGLEC proteins of the invention” or “proteins of the invention”.

Nucleic Acid Molecules of the Invention

Nucleic Acid Molecules Encoding SIGLEC-BMS Proteins

The present invention discloses the discovery of nucleic acid molecules, herein termed Siglec-BMS nucleotide sequences, that encode novel polypeptides having similar structural features shared by proteins in the Siglec subgroup. Structural features shared by the Siglec subgroup include an Ig-like domain which is extracellular and comprises a C-set domain and a V-set domain having an unusual intrasheet disulfide bridge between the B and E strands (A. F. Williams and A. N. Barclay 1988 Annu. Rev. Immunol. 6:381-405; A. F. Williams, et al. 1989 Cold Spring Harbor Symp. Quant. Biol. 54:637-647; L. Pedraza, et al. 1990 J. Cell biol. 111:2651-2661). The nucleotide sequences of Siglec-BMS encode polypeptides each can have two (e.g., -L4,-L5a, and -L5b) to three (e.g., -L3a, -L3b, -L3c, and -L3d) C-set domains.

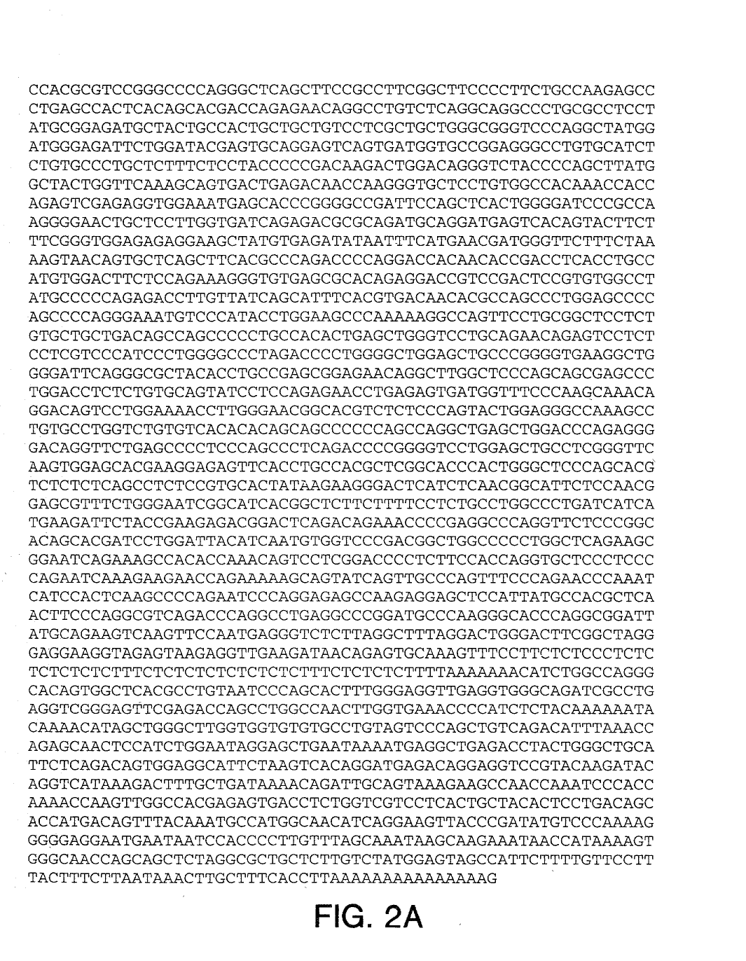

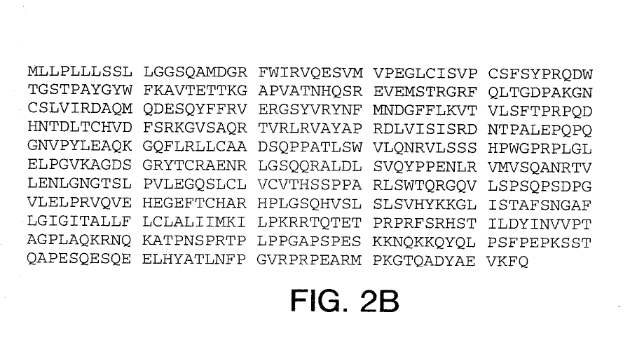

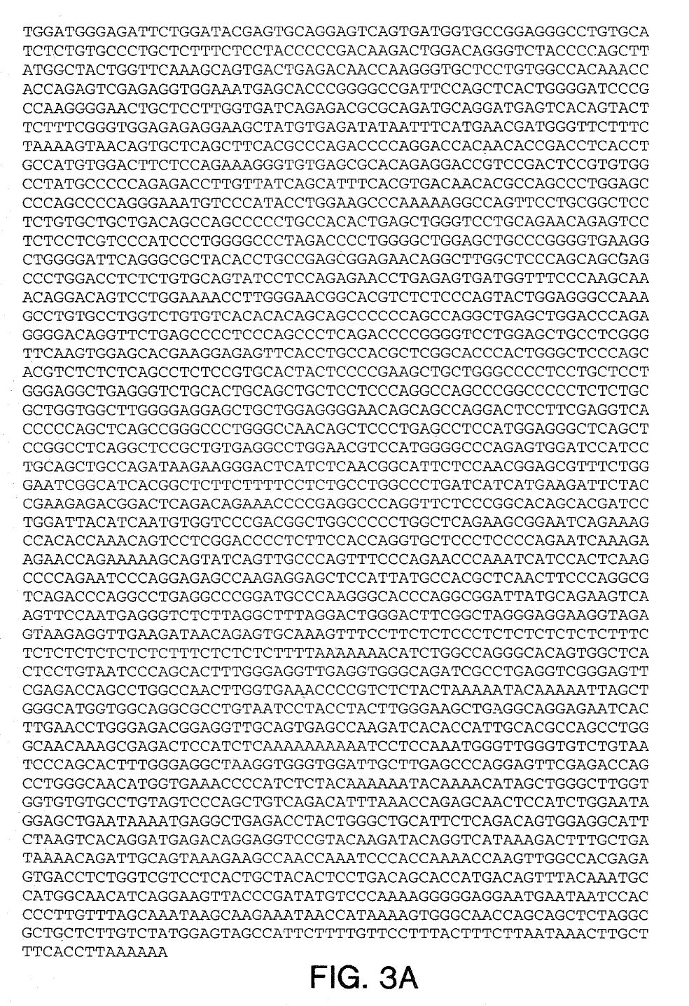

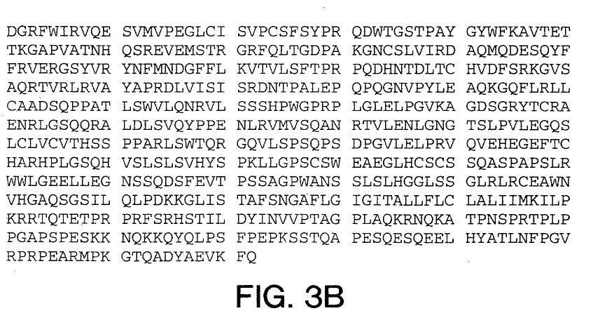

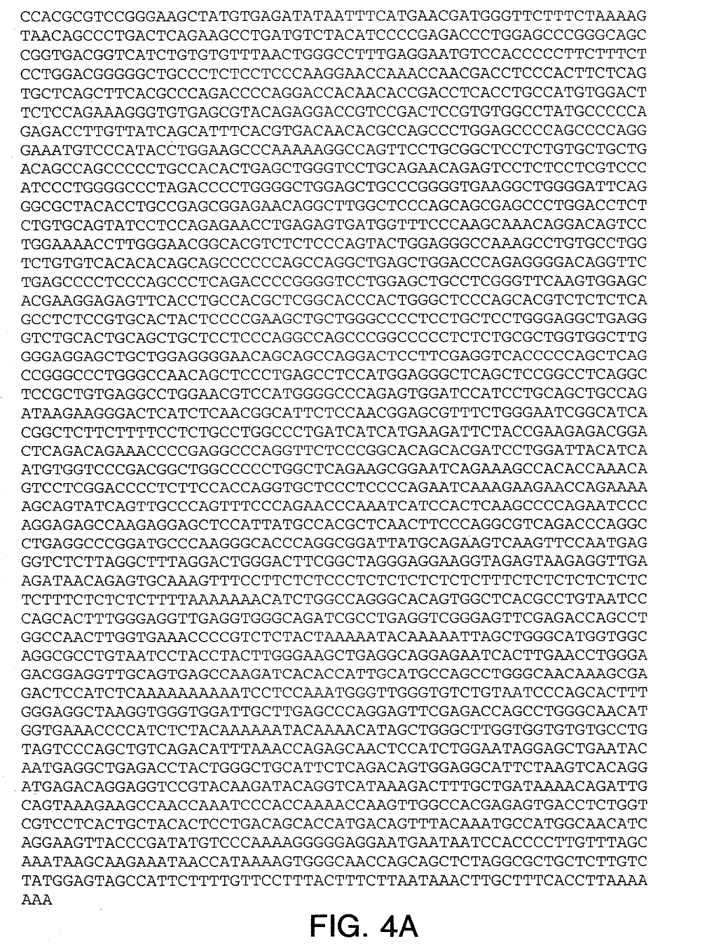

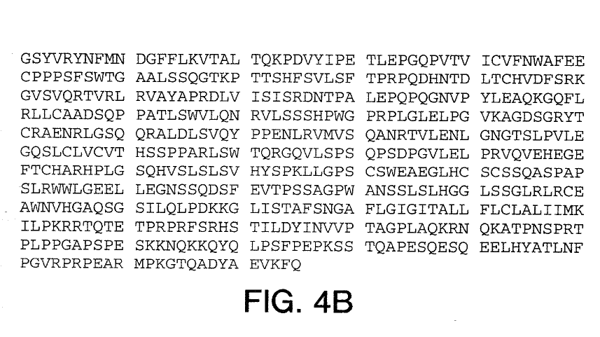

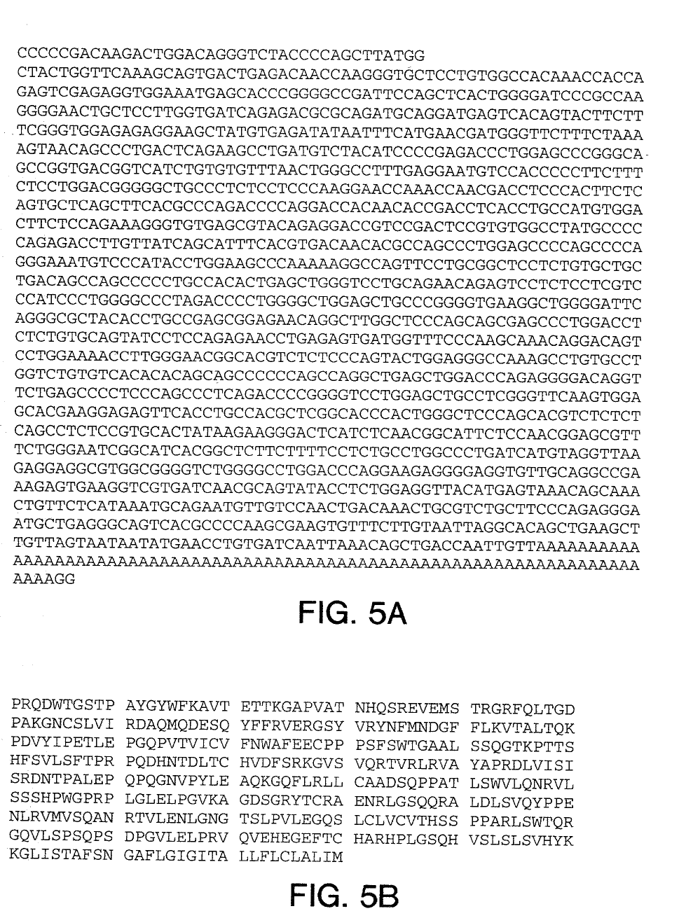

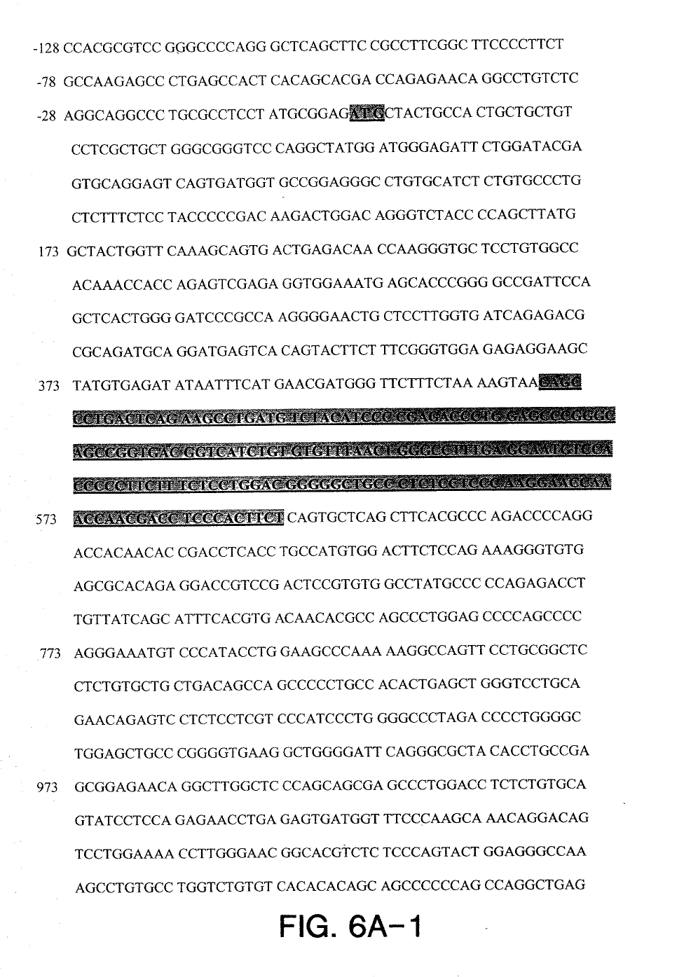

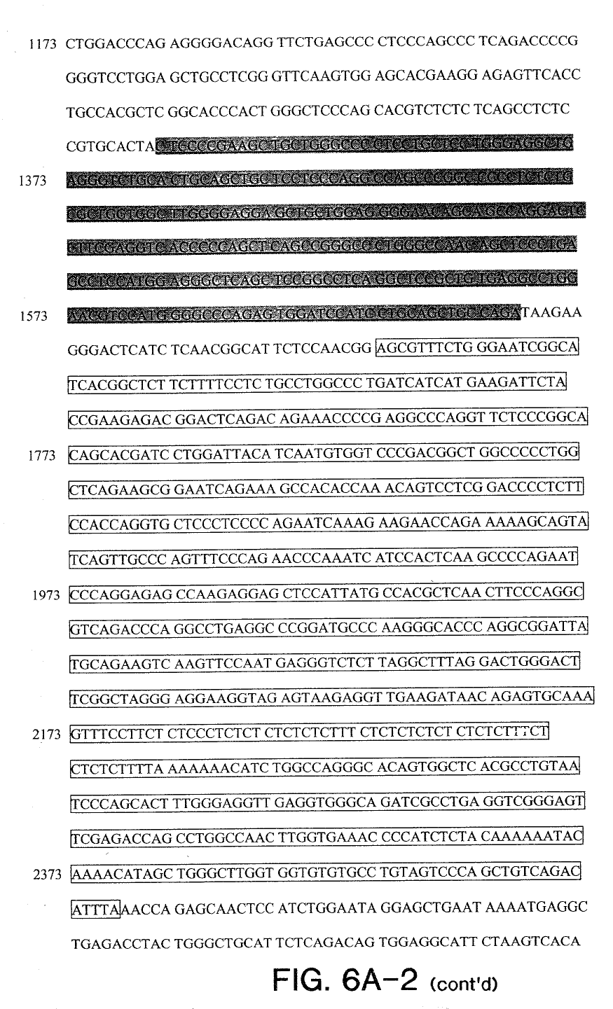

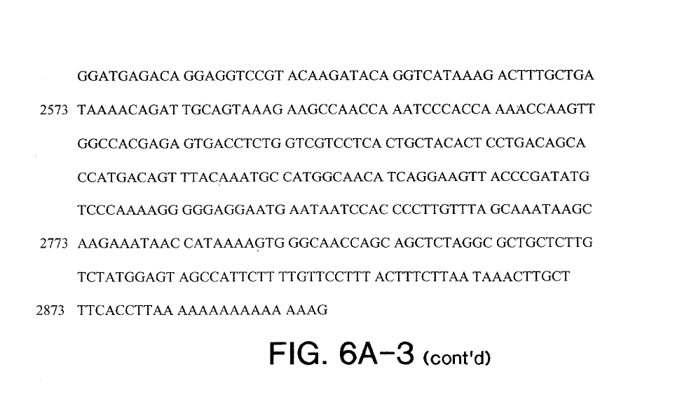

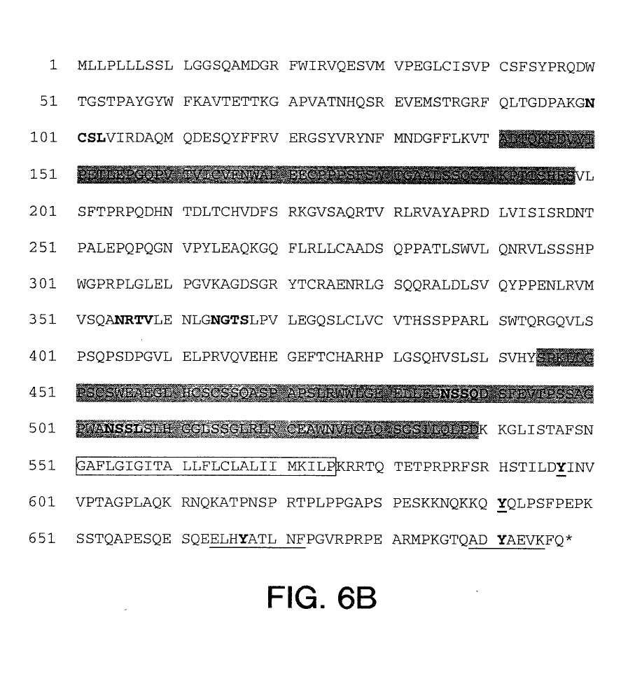

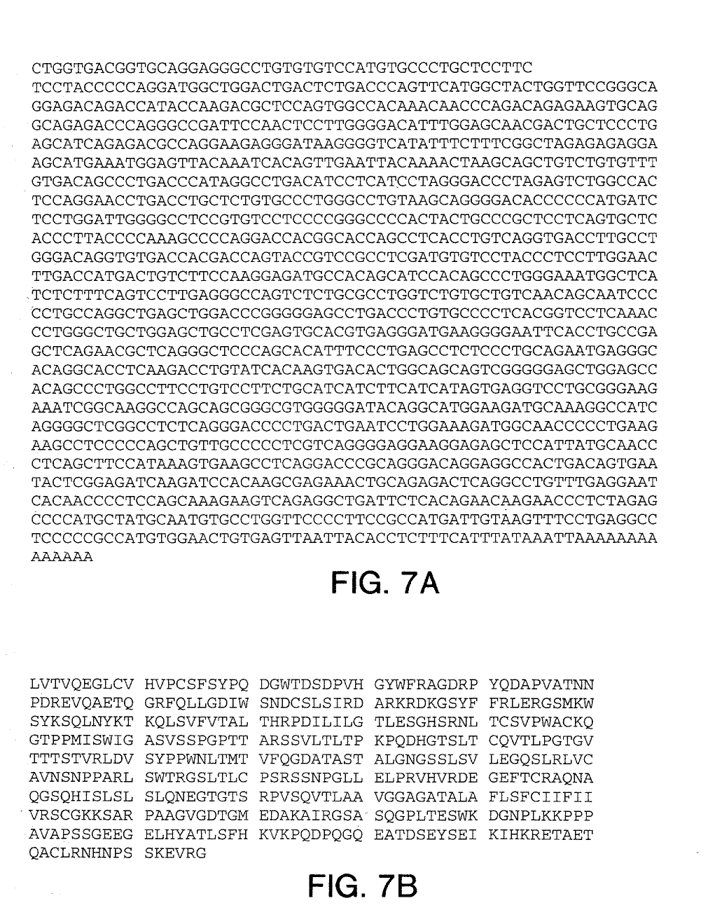

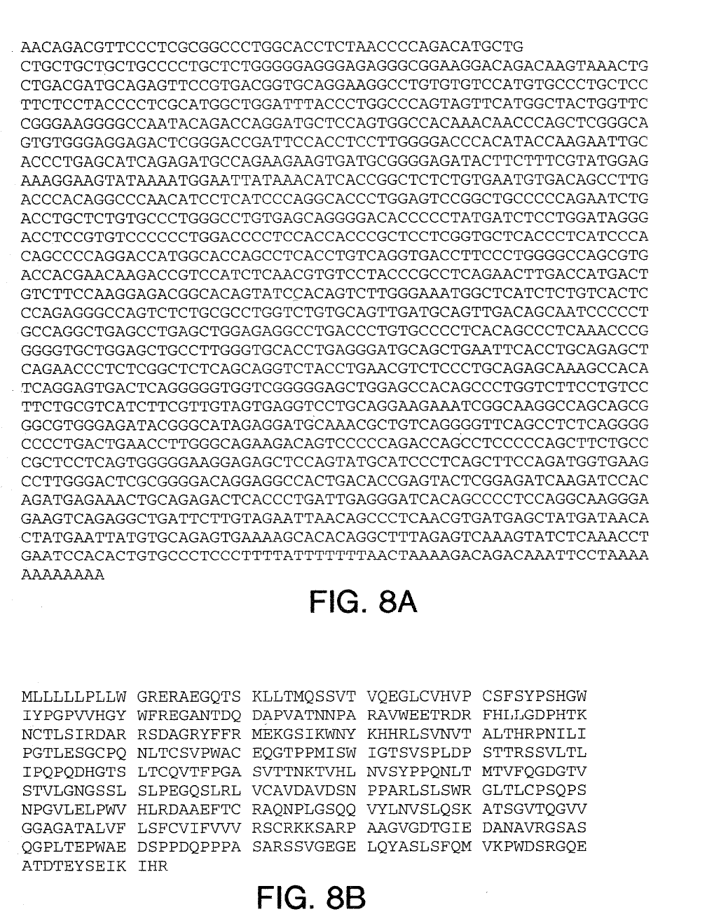

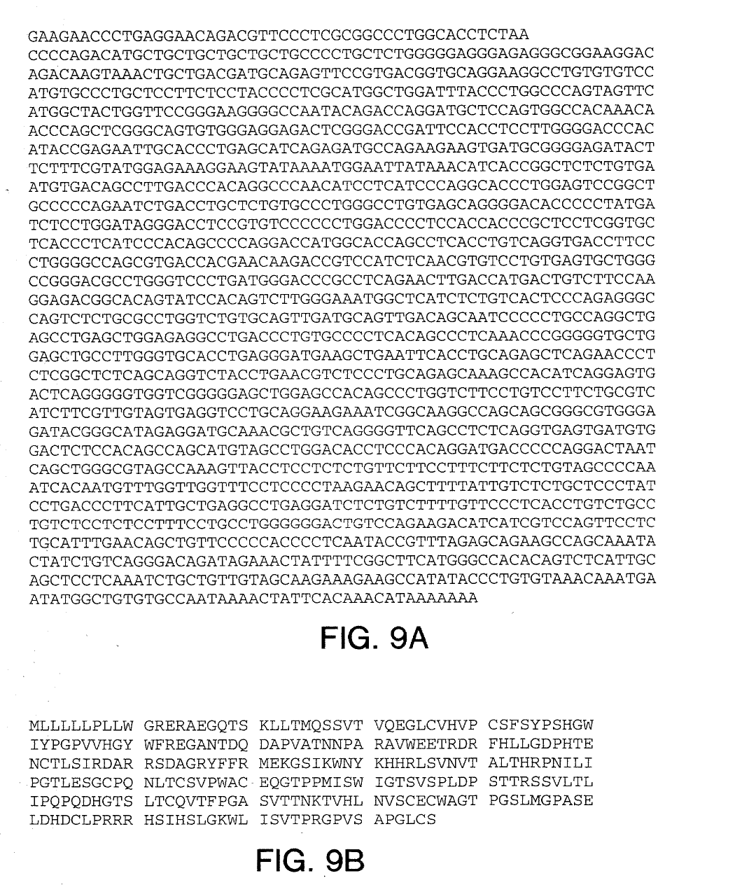

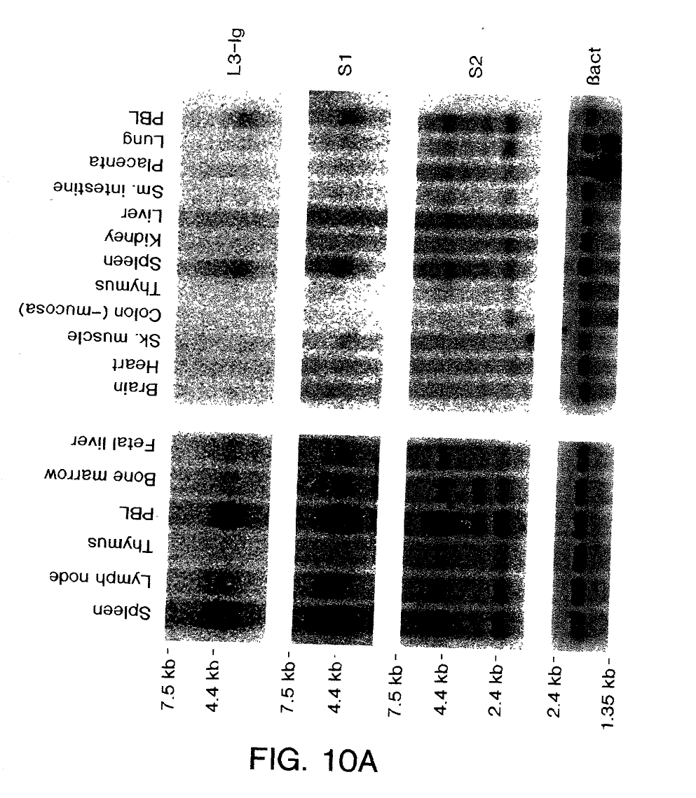

In particular embodiments, novel nucleotide sequences are designated L3a, L3b, L3c, L3d, L4, L5a, L5b, and L3-995-2, as shown shown in FIGS. 2A, 3A, 4A, 5A, 7A, 8A, 9A, and 6A respectively (SEQ ID NOS.:1-7 and 27). These nucleotide sequences encode SIGLEC-BMS proteins and/or fragments thereof, where the encoded proteins exhibit a biological activity, for example, functioning as a cell adhesion molecule. For example, an isolated Siglec nucleic acid encoding L3a is shown in FIG. 2A beginning at codon GGC at position +12 and ending at codon CCA at position +1760. An isolated Siglec nucleic acid encoding L3b is shown in FIG. 3A beginning at codon GAT at position +3 and ending at codon CAA at position +1868. An isolated Siglec nucleic acid encoding L3c is shown in FIG. 4A beginning at codon GGA at position +12 and ending at codon CAA at position +1736. An isolated Siglec nucleic acid encoding L3d is shown in FIG. 5A beginning at codon CCC at position +2 and ending at codon ATG at position +1291. An isolated Siglec nucleic acid encoding L3 is shown in FIG. 6A beginning at codon ATG at position +1 and ending at codon CAA at position +2091. An isolated Siglec nucleic acid encoding L4a is shown in FIG. 7A beginning at codon CTG at position +1 and ending at codon GGC at position +1398. An isolated Siglec nucleic acid encoding L5a is shown in FIG. 8A beginning at codon ATG at position +43 and ending at codon AGA at position +1431. An isolated Siglec nucleic acid encoding L5b is shown in FIG. 9A beginning at codon ATG at position +57 and ending at codon AGT at position +914.

Siglec-BMS-L3, Siglec-BMS-L4 (also referred to herein as L4a), Siglec-BMS-L5a and Siglec-BMS-L5b were collectively deposited on Aug. 10, 2000 with the American Type Culture Collection (ATCC), 10801 University Blvd., Manassas, Va. 20110-2209 under the provisions of the Budapest Treaty, and has been accorded ATCC accession number PTA-2343. The nucleic acid sequences of each of Siglec-BMS-L3, Siglec-BMS-L4 (also referred to herein as L4a), Siglec-BMS-L5a and Siglec-BMS-L5b are provided in FIGS. 6A, 7A, 8A and 9A respectively. These nucleic acid sequences can be easily separated from the collective deposit by standard separation techniques such as hybridization to specific probes or restriction analyses (Maniatis, T., et al., 1989 Molecular Cloning, A Laboratory Manual, Cold Spring Harbor Laboratory, Cold Spring Harbor, N.Y.).

In accordance with the practice of the invention, the nucleotide sequences of the invention may be isolated full-length or partial cDNA molecules or oligomers of the Siglec-BMS sequences. A Siglec-BMS nucleotide sequence can encode all or portions of the signal peptide region, the extracellular domain, the transmembrane domain, and/or the intracellular domain of a SIGLEC-BMS protein.

Isolated Siglec-BMS Sequences

The nucleic acid molecules of the invention are preferably in isolated form, where the nucleic acid molecules are substantially separated from contaminant nucleic acid molecules having sequences other than Siglec-BMS sequences. A skilled artisan can readily employ nucleic acid isolation procedures to obtain isolated Siglec-BMS sequences, see for example Sambrook et al., Molecular Cloning (1989). The present invention also provides for isolated Siglec-BMS sequences generated by recombinant DNA technology or chemical synthesis methods. The present invention also provides nucleotide sequences isolated from various mammalian species including, bovine, ovine, porcine, murine, equine, and preferably, human species.

The isolated nucleic acid molecules include DNA, RNA, DNA/RNA hybrids, and related molecules, nucleic acid molecules complementary to the SIGLEC-BMS encoding sequences or a portion thereof, and those which hybridize to the nucleic acid sequences that encode the SIGLEC-BMS proteins. The preferred nucleic acid molecules have nucleotide sequences identical to or nearly identical (e.g., similar) to the nucleotide sequences disclosed herein. Specifically contemplated are genomic DNA, cDNA, ribozymes, and antisense molecules.

Identical and Variant Siglec-BMS Sequences

The present invention provides isolated nucleic acid molecules having a polynucleotide sequence identical or similar to the Siglec-BMS sequences disclosed herein. Accordingly, the polynucleotide sequences may be identical to a particular Siglec-BMS sequence, as described in SEQ ID NOS.:1-7 or 27. Alternatively, the polynucleotide sequences may be similar to the disclosed sequences.

One embodiment of the invention provides nucleic acid molecules that exhibit sequence identity or similarity with the Siglec-BMS nucleotide sequences, such as molecules that have at least 60% to 99.9% sequence similarity and up to 100% sequence identity with the sequences of the invention as shown in FIGS. 2A, 3A, 4A, 5A, 7A, 8A, 9A and 6A (SEQ ID NOS.:1-7, or 27). A preferred embodiment provides nucleic acid molecules that exhibit between about 75% to 99.9% sequence similarity, a more preferred embodiment provides molecules that have between about 86% to 99.9% sequence similarity, and the most preferred embodiment provides molecules that have 100% sequence identity with the Siglec-BMS sequences of the invention (e.g., SEQ ID NOS.:1-7, or 27).

Differentially Spliced Siglec-BMS Sequences

The nucleic acid molecules of the present invention comprise nucleic acid sequences corresponding to differentially spliced transcripts of Siglec-BMS. In general, a differentially-spliced transcript is a mature RNA transcript that can be generated in a cell by the following steps: (1) the cell transcribes precursor RNA transcripts from an intron-containing gene, where the precursor RNA transcripts include all the intron sequences; (2) the cell splices out different introns from different precursor transcripts, resulting in a heterogeneous population of mature RNA transcripts each having different introns; (3) the cell translates some or all of the differentially-spliced transcripts to generate a heterogeneous population of proteins which are encoded by the same intron-containing gene sequence. Thus, a cell may produce a heterogeneous population of Siglec-BMS RNA transcripts that are related to each other as a result of differential splicing of a common precursor transcript. Furthermore, the SIGLEC-BMS proteins that are translated from the differentially spliced transcripts may have different biological activities.

For example, the polynucleotide sequences of the present invention include introns and can encode three different classes of SIGLEC-BMS proteins: (1) the nucleotide sequences described in FIGS. 2A, 3A, 4A, and 5A (SEQ ID NOS.: 1-4) which represent cDNA clones that are related to each other and correspond to differentially spliced transcripts of Siglec-BMS-L3a, -L3b, -L3c, and -L3d, which encode SIGLEC-BMS proteins -L3 a, -L3b, -L3c, and -L3d respectively (e.g., FIGS. 2b, 3b, 4B, and 5B respectively; SEQ ID NOS.: 8, 9, 10 and 11, respectively); (2) the nucleotide sequence described in FIG. 7A (SEQ ID NO.: 5) which represents a cDNA clone that corresponds to a differentially spliced transcript of Siglec-BMS-L4a which encodes SIGLEC-BMS-4a protein (FIG. 7B; SEQ ID NOS.: 12); (3) the nucleotide sequences described by FIGS. 8A and 9A (SEQ ID NOS.: 6-7) which represent cDNA clones that are related to each other and correspond to differentially spliced transcripts of Siglec-BMS-L5a, and -L5b, which encode SIGLEC-BMS-5a and -5b proteins, respectively (e.g., FIGS. 8B and 9b; SEQ ID NOS.:13 and 14, respectively). The invention also provides nucleic acid molecules having the nucleotide sequence of Siglec-BMS-L3-995-2 (FIG. 6A; SEQ ID NO.:27), which represents a hybrid construct of full-length Siglec-BMS-L3 cDNA (Example 14, infra).

Complementary Sequences

The invention also provides nucleic acid molecules that are complementary to the sequences as described in FIGS. 2A, 3A, 4A, 5A, 7A, 8A, 9A, and 6A (SEQ ID NO: 1-7, and 27) (preferably, the coding sequences excluding the vector sequences therein). Complementarity may be full or partial. When it is fully complementary that means compementarity to the entire sequence as described in SEQ ID NO:1-7, and 27. When it is partially complementary that means complementarity to only portions of sequences as described in SEQ ID NO: 1-7, and 27.

Nucleotide Sequences Which Hybridize to Siglec-BMS Sequences

The present invention further provides nucleotide sequences that selectively hybridize to Siglec-BMS nucleotide sequences (e.g., SEQ ID NO.: 1-7, or 27) under high stringency hybridization conditions. Typically, hybridization under standard high stringency conditions will occur between two complementary nucleic acid molecules that differ in sequence complementarity by about 70% to about 100%. It is readily apparent to one skilled in the art that the high stringency hybridization between nucleic acid molecules depends upon, for example, the degree of identity, the stringency of hybridization, and the length of hybridizing strands. The methods and formulas for conducting high stringency hybridizations are well known in the art, and can be found in, for example, Sambrook, et al., Molecular Cloning (1989).

In general, stringent hybridization conditions are those that: (1) employ low ionic strength and high temperature for washing, for example, 0.015M NaCl/0.0015M sodium titrate/0.1% SDS at 50 degrees C.; or (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42 degrees C.

Another example of stringent conditions include the use of 50% formamide, 5× SSC (0.75M NaCl, 0.075 M sodium citrate), 50 mM sodium phosphate (pH 6.8), 0.1% sodium pyrophosphate, 5× Denhardt's solution, sonicated salmon sperm DNA (50 mg/ml), 0.1% SDS, and 10% dextran sulfate at 42 degrees C., with washes at 42 degrees C. in 0.2× SSC and 0.1% SDS. A skilled artisan can readily determine and vary the stringency conditions appropriately to obtain a clear and detectable hybridization signal.

Fragments of Siglec-BMS Sequences

The invention further provides nucleic acid molecules having fragments of the Siglec-BMS sequences of the invention, such as a portion of the Siglec-BMS sequences disclosed herein (as shown in SEQ ID NO.:1-7, 15, and 27). The size of the fragment will be determined by its intended use. For example, if the fragment is chosen to encode a SIGLEC-BMS extracellular domain, then the skilled artisan shall select the polynucleotide fragment that is large enough to encode this domain(s). If the fragment is to be used as a nucleic acid probe or PCR primer, then the fragment length is chosen to obtain a relatively small number of false positives during a probing or priming procedure. Alternatively, a fragment of the Siglec-BMS sequence may be used to construct a recombinant fusion gene having a Siglec-BMS sequence fused to a non-Siglec-BMS sequence, such as a human immunoglobulin or a GST sequence.

The nucleic acid molecules, fragments thereof, and probes and primers of the present invention are useful for a variety of molecular biology techniques including, for example, hybridization screens of libraries, or detection and quantification of mRNA transcripts as a means for analysis of gene transcription and/or expression. Preferably, the probes and primers are DNA. A probe or primer length of at least 15 base pairs is suggested by theoretical and practical considerations (Wallace, B. and Miyada, G. 1987 in: “Oligonucleotide Probes for the Screening of Recombinant DNA Libraries” in: Methods in Enzymology, 152:432-442, Academic Press).

Fragments of Siglec-BMS nucleotide sequences that are particularly useful as selective hybridization probes or PCR primers can be readily identified from the Siglec-BMS nucleotide sequences, using art-known methods. For example, sets of PCR primers that detect the portion of Siglec-BMS transcripts that encode the extracellular domain of a SIGLEC protein can be made by the PCR method described in U.S. Pat. No. 4,965,188. The probes and primers of this invention can be prepared by methods well known to those skilled in the art (Sambrook, et al. supra). In a preferred embodiment the probes and primers are synthesized by chemical synthesis methods (ed: Gait, M. J. 1984 Oligonucleotide Synthesis, IRL Press, Oxford, England).

One embodiment of the present invention provides nucleic acid primers that are complementary to Siglec-BMS sequences, which allow the specific amplification of nucleic acid molecules of the invention or of any specific portions thereof. Another embodiment provides nucleic acid probes that are complementary for selectively or specifically hybridizing to the Siglec-BMS sequences or to any portion thereof, e.g., a all or portion of the extracellular domain.

Fusion Genes

The present invention provides fusion genes, which include a Siglec-BMS sequence fused (e.g., linked or joined) to a non-Siglec-BMS sequence such as, for example, a HIS-tag sequence, to facilitate isolation and/or purification of the expressed SIGLEC-BMS gene product (Kroll, D. J., et al., 1993 DNA Cell Biol 12:441-53), or a GST, or a human immunoglobulin sequence. The preferred fusion gene comprises a Siglec-BMS sequence operatively linked to a non-Siglec-BMS sequence, such as, for example a Siglec-BMS sequence fused in-frame with a non-Siglec-BMS sequence.

Alternatively, the fusion genes of the invention include a Siglec-BMS sequence fused to a Siglec-BMS sequence isolated from a different mammalian source. For example, the human Siglec-BMS sequences, disclosed herein, can be fused to a Siglec-BMS sequence isolated from a different human or a different mammalian species.

Codon Usage Variants Encoding SIGLEC-BMS Proteins

The present invention provides isolated codon-usage variants that differ from the disclosed Siglec-BMS nucleotide sequences, yet do not alter the predicted SIGLEC-BMS polypeptide sequence or biological activity. For example, a number of amino acids are designated by more than one triplet. Codons that specify the same amino acid, or synonyms may occur due to degeneracy in the genetic code. Examples include nucleotide codons CGT, CGG, CGC, and CGA encoding the amino acid, arginine (R); or codons GAT, and GAC encoding the amino acid, aspartic acid (D). Thus, a protein can be encoded by one or more nucleic acid molecules that differ in their specific nucleotide sequence, but still encode protein molecules having identical sequences. The amino acid coding sequence is as follows:

| | | One Letter | |
|---|---|---|---|
| Amino Acid | Symbol | Symbol | Codons |
| Alanine | Ala | A | GCU, GCC, GCA, GCG |
| Cysteine | Cys | C | UGU, UGC |
| Aspartic Acid | Asp | D | GAU, GAC |
| Glutamic Acid | Glu | E | GAA, GAG |
| Phenylalanine | Phe | F | UUU, UUC |
| Glycine | Gly | G | GGU, GGC, GGA, GGG |
| Histidine | His | H | CAU, CAC |
| Isoleucine | Ile | I | AUU, AUC, AUA |
| Lysine | Lys | K | AAA, AAG |
| Leucine | Leu | L | UUA, UUG, CUU, CUC, CUA, CUG |
| Methionine | Met | M | AUG |
| Asparagine | Asn | N | AAU, AAC |
| Proline | Pro | P | CCU, CCC, CCA, CCG |
| Glutamine | Gln | Q | CAA, CAG |
| Arginine | Arg | R | CGU, CGC, CGA, CGG, AGA, AGG |
| Serine | Ser | S | UCU, UCC, UCA, UCG, AGU, AGC |
| Threonine | Thr | T | ACU, ACC, ACA, ACG |
| Valine | Val | V | GUU, GUC, GUA, GUG |
| Tryptophan | Trp | W | UGG |
| Tyrosine | Tyr | Y | UAU, UAC |

The codon-usage variants may be generated by recombinant DNA technology. Codons may be selected to optimize the level of production of the Siglec-BMS transcript or SIGLEC-BMS polypeptide in a particular prokaryotic or eukaryotic expression host, in accordance with the frequency of codon utilized by the host cell. Alternative reasons for altering the nucleotide sequence encoding a SIGLEC-BMS polypeptide include the production of RNA transcripts having more desirable properties, such as an extended half-life or increased stability. A multitude of variant Siglec-BMS nucleotide sequences that encode the respective SIGLEC-BMS polypeptide may be isolated, as a result of the degeneracy of the genetic code. Accordingly, the present invention provides selecting every possible triplet codon to generate every possible combination of nucleotide sequences that encode the disclosed SIGLEC-BMS polypeptides, or that encode polypeptides having the biological activity of the SIGLEC-BMS polypeptides. This particular embodiment provides isolated nucleotide sequences that vary from the sequences as described in SEQ ID NOS.: 1-7, or 27, such that each variant nucleotide sequence encodes a polypeptide having sequence identity with the amino acid sequences, as described in FIGS. 2B, 3B, 4B, 5B, 7B, 8B, 9B, or 6B (SEQ ID NOs.: 8-14, or 28), respectively.

Allelic Forms of Siglec-BMS Sequences

The present invention contemplates alternative allelic forms of the Siglec-BMS nucleotide sequences. These alternative allelic forms can be isolated from different subjects of the same species.

Typically, isolated allelic forms of naturally-occurring gene sequences include wild-type and mutant alleles. A wild-type Siglec-BMS gene sequence will encode a SIGLEC-BMS protein having normal SIGLEC-BMS biological activity, such as, for example, function as a cell adhesion molecule. A mutant Siglec-BMS gene sequence may encode a SIGLEC-BMS protein having an activity not found in normal SIGLEC-BMS proteins, such as, for example, not functioning as a cell adhesion molecule. Alternatively, a mutant Siglec-BMS gene sequence may encode a SIGLEC-BMS protein having normal activity.

It will be appreciated by one skilled in the art that variations in one or more nucleotides (up to about 3-4% of the nucleotides) of the nucleic acids encoding peptides having the activity of a SIGLEC-BMS molecule may exist among individuals within a population due to natural allelic variation. Any and all such nucleotide variations and resulting amino acid polymorpism are within the scope of the invention.

Polymorphic Forms of Siglec-BMS Sequences

The present invention provides nucleotide sequences of particular polymorphic forms of Siglec-BMS, as described in SEQ ID NOS.: 1-7, or 27. Typically, isolated polymorphic forms of naturally-occurring gene sequences are isolated from different subjects of the same species. The polymorphic forms include sequences having one or more nucleotide substitutions that may or may not result in changes in the amino acid codon sequence. These substitutions may result in a wild-type Siglec-BMS gene that encodes a protein having the biological activity of wild-type SIGLEC-BMS proteins, or encodes a mutant polymorphic form of the SIGLEC-BMS protein having a different or null activity.

Derivative Nucleic Acid Molecules

The nucleic acid molecules of the invention also include derivative nucleic acid molecules which differ from DNA or RNA molecules, and anti-sense molecules. Derivative molecules include peptide nucleic acids (PNAs), and non-nucleic acid molecules including phosphorothioate, phosphotriester, phosphoramidate, and methylphosphonate molecules, that bind to single-stranded DNA or RNA in a base pair-dependent manner (Zamecnik, P. C., et al., 1978 Proc. Natl. Acad. Sci. 75:280284; Goodchild, P. C., et al., 1986 Proc. Natl. Acad. Sci. 83:4143-4146). Peptide nucleic acid molecules comprise a nucleic acid oligomer to which an amino acid residue, such as lysine, and an amino group have been added. These small molecules, also designated anti-gene agents, stop transcript elongation by binding to their complementary (template) strand of nucleic acid (Nielsen, P. E., et al., 1993 Anticancer Drug Des 8:53-63). Reviews of methods for synthesis of DNA, RNA, and their analogues can be found in: Oligonucleotides and Analogues, eds. F. Eckstein, 1991, IRL Press, New York; Oligonucleotide Synthesis, ed. M. J. Gait, 1984, IRL Press, Oxford, England. Additionally, methods for antisense RNA technology are described in U.S. Pat. Nos. 5,194,428 and 5,110,802. A skilled artisan can readily obtain these classes of nucleic acid molecules using the herein described Siglec-BMS polynucleotide sequences, see for example Innovative and Perspectives in Solid Phase Synthesis (1992) Egholm, et al. pp 325-328 or U.S. Pat. No. 5,539,082.

RNA Encoding Siglec-BMS Polypeptides

The present invention provides nucleic acid molecules that encode SIGLEC-BMS proteins. In particular, the RNA molecules of the invention may be isolated full-length or partial mRNA molecules or RNA oligomers that encode the SIGLEC-BMS proteins. The RNA molecules of the invention also include antisense RNA molecules, peptide nucleic acids (PNAs), or non-nucleic acid molecules such as phosphorothioate derivatives, that specifically bind in a base-dependent manner to the sense strand of DNA or RNA, having the Siglec-BMS sequences, in a base-pair manner. A skilled artisan can readily obtain these classes of nucleic acid molecules using the Siglec-BMS sequences described herein.

Nucleic Acid Molecules Labeled With A Detectable Marker

Embodiments of the Siglec-BMS nucleic acid molecules of the invention include DNA and RNA primers, which allow the specific amplification of Siglec-BMS sequences, or of any specific parts thereof, and probes that selectively or specifically hybridize to Siglec-BMS sequences or to any part thereof. The nucleic acid probes can be labeled with a detectable marker. Examples of a detectable marker include, but are not limited to, a radioisotope, a fluorescent compound, a bioluminescent compound, a chemiluminescent compound, a metal chelator or an enzyme. Technologies for generating labeled DNA and RNA probes are well known, see, for example, Sambrook et al., in Molecular Cloning (1989).

Proteins and Polypeptides of the Invention

The invention also provide novel SIGLEC-BMS proteins. One embodiment of a SIGLEC-BMS protein comprises an amino acid sequence beginning with Ala141 and ending with Ser198 as shown in FIG. 6B (SEQ ID NO:28). Another embodiment of a SIGLEC-BMS protein comprises an amino acid sequence beginning with Ala141 and ending with Ser198 as shown in FIG. 6B (SEQ ID NO:28) and is encoded by a nucleic acid molecule that hybridizes, under stringent conditions to a nucleic acid molecule that is complementary to the nucleic acid as shown in any one of SEQ. ID NOS. 1-7 and 27. Another embodiment of a SIGLEC protein comprises an amino acid sequence that is encoded by a nucleic acid molecule that hybridizes, under stringent conditions to a nucleic acid molecule that is complementary to the nucleic acid as shown in any one of SEQ ID NOS. 1-7 and 27. Particular embodiments of the novel proteins of the invention sequences include SIGLEC-BMS -L3a, -L3b, -L3c, -L3d, -L4,-L5a, -L5b, and -L3-995-2, (shown in SEQ ID NOS.:8-14 and 28, respectively). SIGLEC-BMS proteins may be embodied in many forms, preferably in isolated or purified form.

The SIGLEC-BMS proteins may be isolated from mammalian species including, bovine, ovine, porcine, murine, equine, and preferably human. Alternatively, purified SIGLEC-BMS proteins may be generated by synthetic, semi-synthetic, or recombinant methods.

A skilled artisan can readily employ standard isolation and purification methods to obtain isolated and/or purified SIGLEC proteins (Marchak, D. R., et al., 1996 in: Strategies for Protein Purification and Characterization, Cold Spring Harbor Press, Plainview, N.Y.). The nature and degree of isolation and purification will depend on the intended use. For example, purified SIGLEC-BMS protein molecules will be substantially free of other proteins or molecules that impair the binding of SIGLEC-BMS to antibodies or other ligands. Embodiments of the SIGLEC-BMS proteins include a purified SIGLEC-BMS protein or fragments thereof, having the biological activity of a SIGLEC-BMS protein. In one form, such purified SIGLEC-BMS proteins, or fragments thereof, retain the ability to bind antibody or other ligand.

In a cell, the Siglec-BMS gene sequences are predicted to include signal peptide sequences and introns, therefore it is expected that the cell will produce various forms of a particular SIGLEC-BMS protein as a result of post-translational modification. For example, various forms of isolated, SIGLEC-BMS proteins may include: precursor forms that include the signal peptide, mature forms that lack the signal peptide, and different mature forms of a SIGLEC-BMS protein that result from post-translational events such as intramolecular cleavage.

The present invention provides isolated and purified proteins, polypeptides, and fragments thereof, having an amino acid sequence identical to the predicted sequence of the SIGLEC-BMS sequences disclosed herein. Accordingly, the amino acid sequences may be identical to a particular SIGLEC-BMS sequence, as described in any of SEQ ID NOS.: 8-14, or 28.

The present invention also includes proteins having sequence variations from the predicted SIGLEC-BMS protein sequences disclosed herein (e.g., FIGS. 2B, 3B, 4B, 5B, 7B, 8B, 9B, and 6B; SEQ ID NOS.: 8-14, or 28). For example, the proteins having the variant sequences include allelic variants, mutant variants, conservative substitution variants, and SIGLEC-BMS proteins isolated from other mammalian organisms. The amino acid sequences may be similar to the disclosed sequences. For example, two protein sequences are considered to be similar to each other when the percentage of amino acid residues that differ between the two sequences is between about 60% to 99.99%.

The present invention encompasses mutant alleles of Siglec-BMS that encode mutant forms of SIGLEC-BMS proteins having one or more amino acid substitutions, insertions, deletions, truncations, or frame shifts. Such mutant forms of proteins typically do not exhibit the same biological activity as wild-type proteins. The mutant alleles of Siglec-BMS may or may not encode a SIGLEC-BMS protein having the same biological activity as wild-type SIGLEC-BMS proteins, such as functioning as a cell adhesion molecule.

Another variant of SIGLEC-BMS proteins may have amino acid sequences that differ by one or more amino acid substitutions. The variant may have conservative amino acid changes, where a substituted amino acid has similar structural or chemical properties, such as replacement of leucine with isoleucine. Alternatively, a variant may have nonconservative amino acid changes, such as replacement of a glycine with a tryptophan. Similar minor variations may also include amino acid deletions or insertions, or both. Guidance in determining which and how many amino acid residues may be substituted, inserted or deleted may be found using computer programs well known in the art, for example, DNASTAR software.

Conservative amino acid substitutions can frequently be made in a protein without altering either the conformation or the biological activity of the protein. Such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered conservative in particular environments.

The proteins of the invention exhibit the biological activities of a SIGLEC-BMS protein, such as, for example, the ability to elicit the generation of antibodies that specifically bind an epitope associated with SIGLEC-BMS proteins. Accordingly, the SIGLEC-BMS protein, or any oligopeptide thereof, is capable of inducing a specific immune response in appropriate animals or cells, and/or binding with specific antibodies.

The SIGLEC-BMS Extracellular Domains

The present invention provides isolated proteins having the extracellular and/or the cytoplasmic domains of the SIGLEC-BMS proteins.

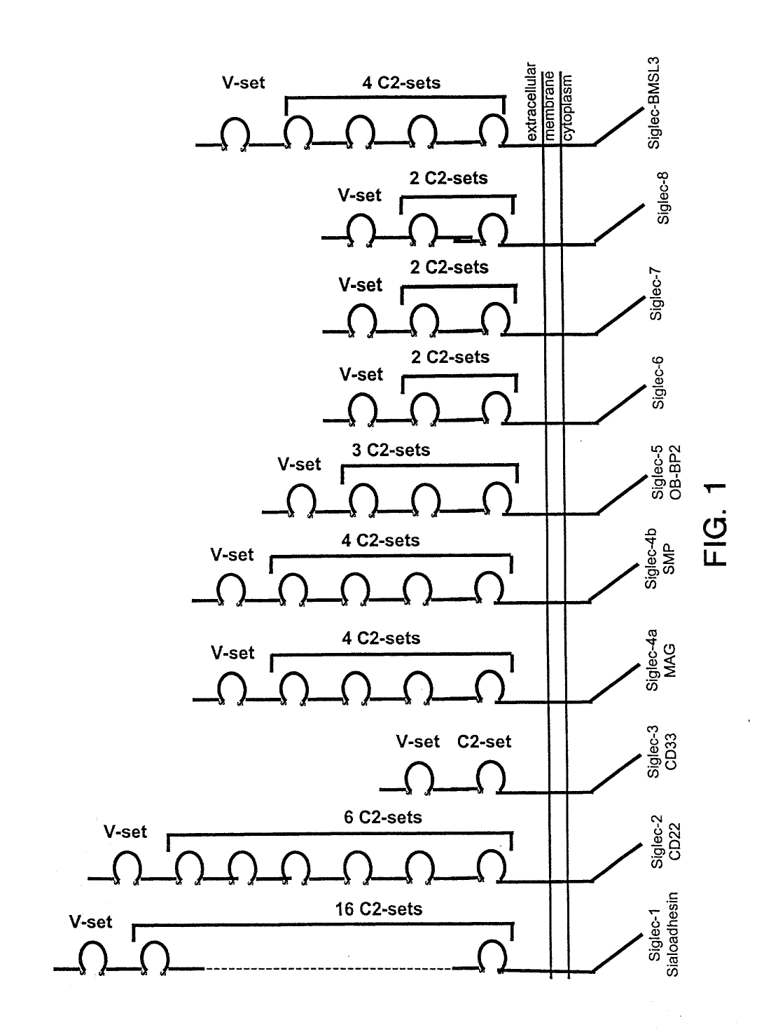

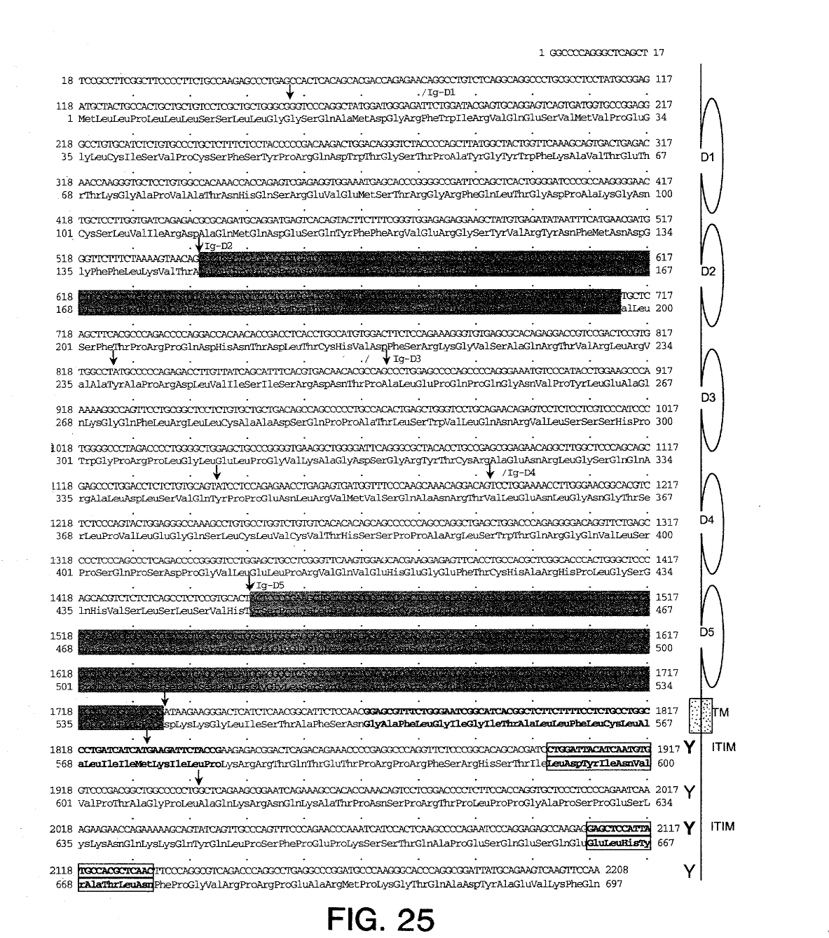

The extracellular domain of SIGLEC-BMS proteins comprises multiple Ig-like domains (FIG. 1). The full-length SIGLEC-BMS protein (SIGLEC-BMS-L3, also designated as SIGLEC-BMS-L3-995-2) contains five Ig-like domains, Ig-D1 (V-set, Ser14 through Thr140), Ig-D2 (C-set, Ala141 through Ala235), Ig-D3 (C-set, Ala252 through Gln341), Ig-D4 (C-set, Val358 through His443), and Ig-D5 (C-set, Tyr444 through Pro538) (FIG. 25).

The extracellular domains of known Siglec proteins (e.g., CD33) are postulated to bind with sialyated cell surface glycans (Kelm, S., et al., 1996 supra; Kelm, S., et al., 1998 supra; Vinson, M., et al., 1996 supra) and mediate cell adhesion or cell signaling. To determine if the extracellular domain of SIGLEC-BMS proteins bind with sialyated cell surface glycans, various protein binding analyses may be performed. The binding analyses include methods, such as fluorescence-activated cell sorting (e.g., FACs), ELISA analysis, and cell binding analysis.

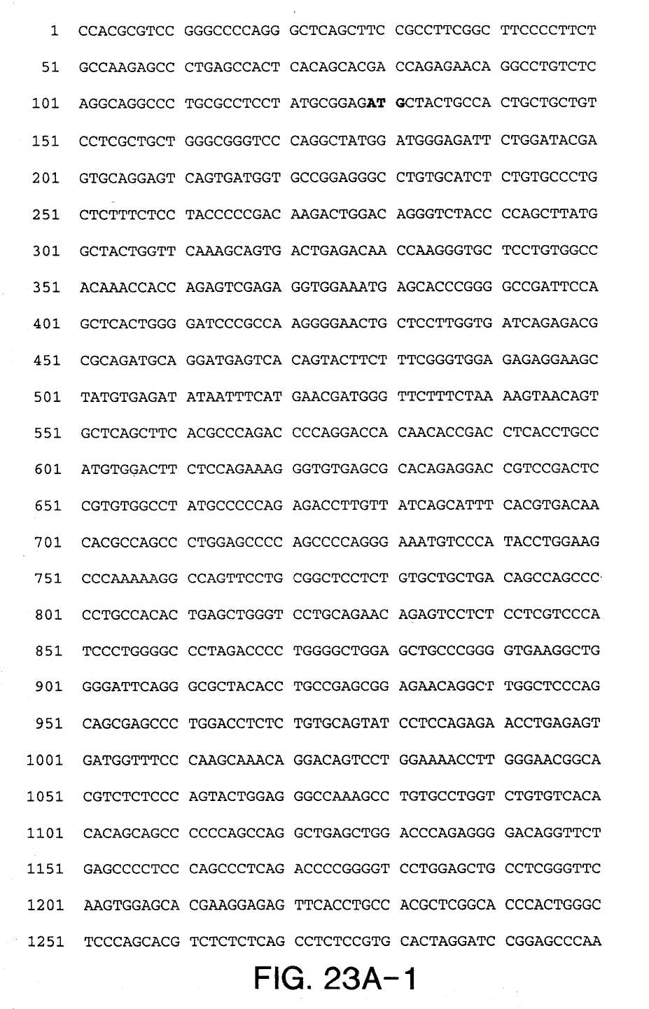

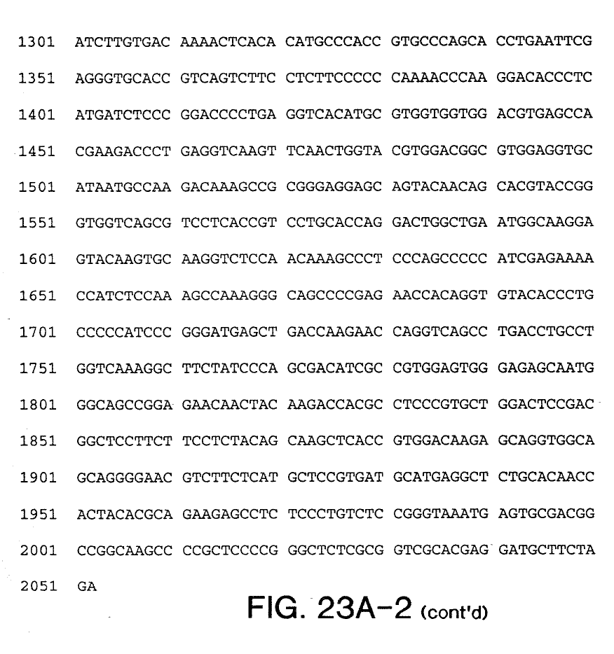

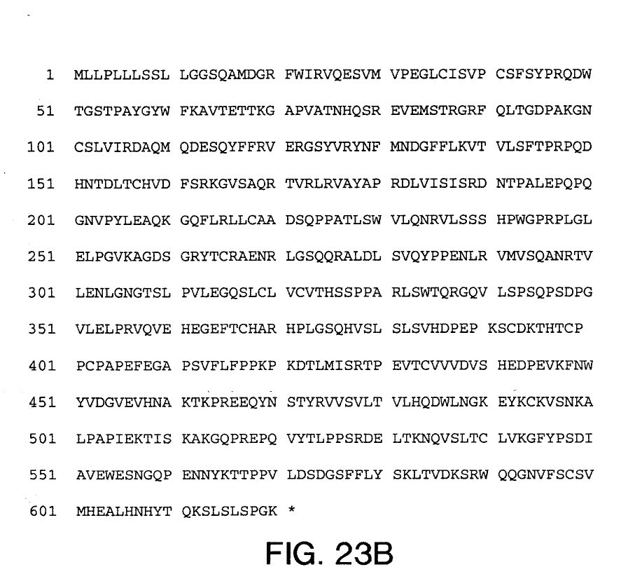

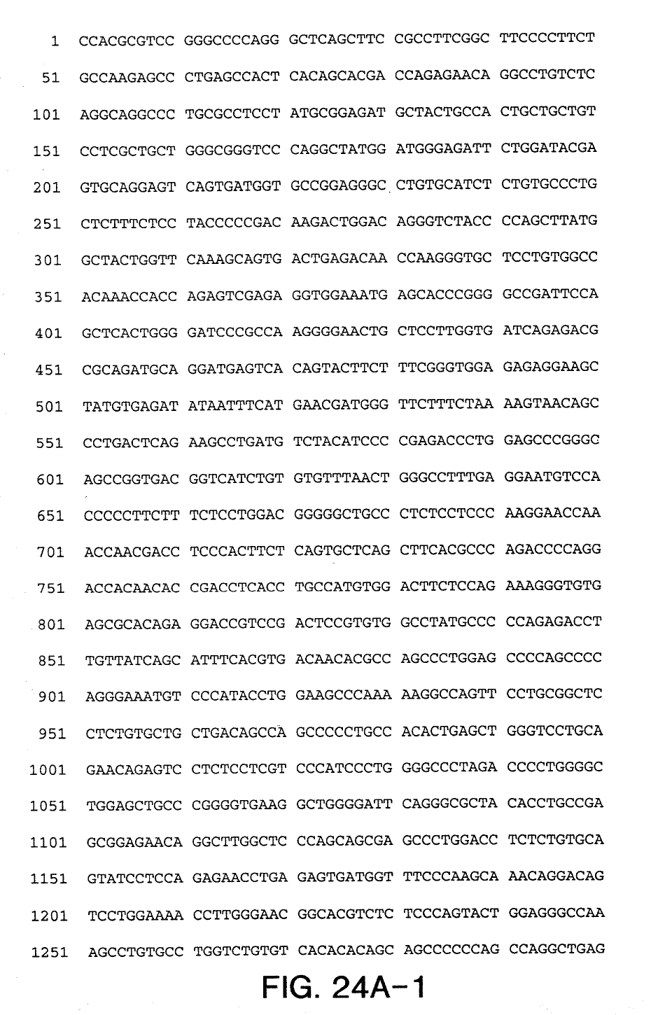

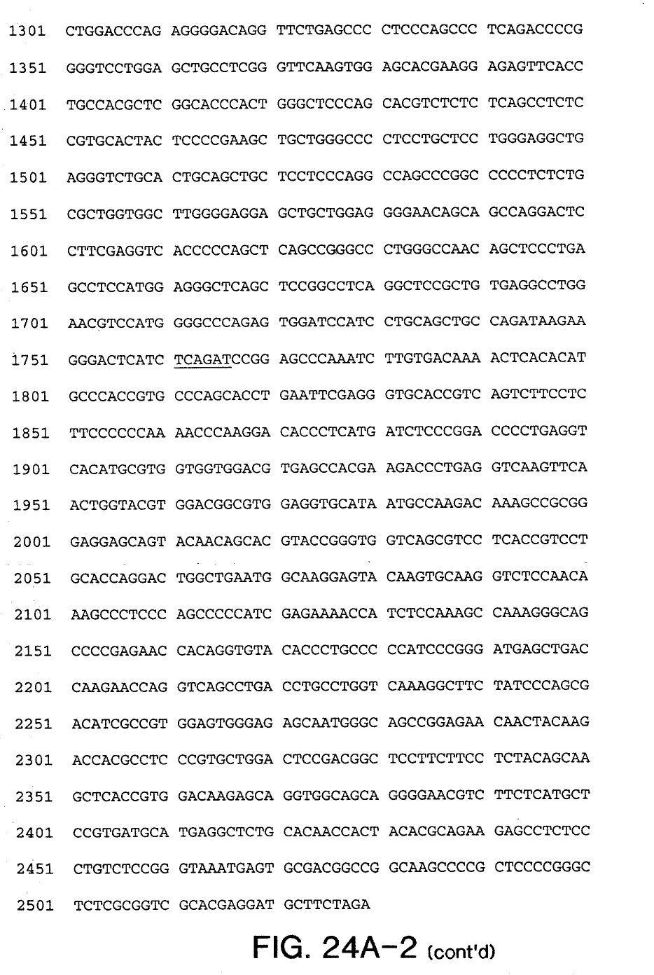

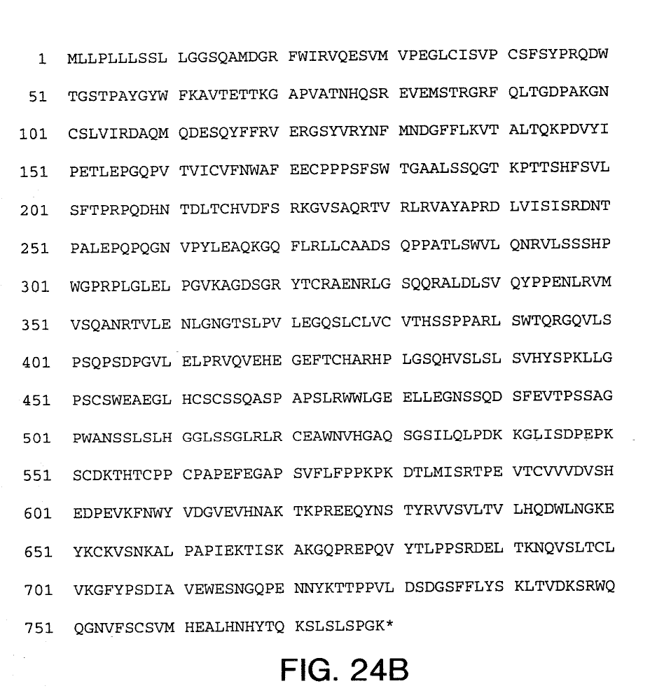

The FACs analyses are conducted using full-length SIGLEC-BMS proteins, fragments thereof, a SIGLEC-BMS fusion protein, or a mutant SIGLEC-BMS protein. The preferred method includes using polypeptides having the extracellular domains of SIGLEC-BMS, such as the fusion proteins described in FIGS. 23 or 24. The binding studies are performed by reacting populations of mixed white blood cells, or hemopoietic cell lines with polypeptides having the extracellular domains of the SIGLEC-BMS proteins.

The binding specificity of SIGLEC-BMS proteins is also determined using a solid support method. The SIGLEC-BMS proteins are immobilized on a solid support, such as an ELISA plate. The SIGLEC-BMS proteins used include full-length SIGLEC-BMS proteins, fragments thereof, a SIGLEC-BMS fusion protein, or mutant SIGLEC-BMS protein. The cells are pre-treated with sialidase. The immobilized proteins are reacted with cells or cell lines including: mixed white blood cells, mixed granulocytes, B cells, T cells, NK cells, and monocytes.

Alternatively, the binding specificity of the SIGLEC-BMS proteins is analyzed by reacting various cell types or cell lines with cells that express the SIGLEC-BMS proteins of the invention. The protein-expressing cells are generated using methods well known in the art, including methods that result in transient or long-term expression of the SIGLEC-BMS proteins. The protein-expressing cells may be mammalian, insect, plant, bacterial, or yeast cells. The protein-expressing cells may express full-length SIGLEC-BMS proteins, or a fragment thereof, a SIGLEC-BMS fusion protein, or a mutant SIGLEC-BMS protein. The protein-expressing cells are reacted with various cell types or cell lines, including: mixed white blood cells, mixed granulocytes, B cells, T cells, NK cells, and monocytes. The reacting cells are pre-treated with sialidase.

The SIGLEC-BMS Cytoplasmic Domains

The cytoplasmic domain of known Siglec proteins have tyrosine residues within ITAM or ITIM motifs which mediate phosphorylation within a cell. For example, the cytoplasmic tail of Siglec-3 (e.g., CD33) includes two ITIM motifs that recruit SHP-1 and SHP-2 upon phosphorylation (Taylor, V., et al., 1999 supra).

To determine if the cytoplasmic tail domain of the SIGLEC-BMS proteins mediates phosphorylation, various methods may be performed. The methods include kinase assays.

The kinase assays are conducted by reacting SIGLEC-BMS proteins with kinases which provide the phosphorylation activity. The kinases are reacted with SIGLEC-BMS proteins, including full-length SIGLEC-BMS proteins, fragments thereof, a SIGLEC-BMS fusion protein, or a mutant SIGLEC-BMS protein.

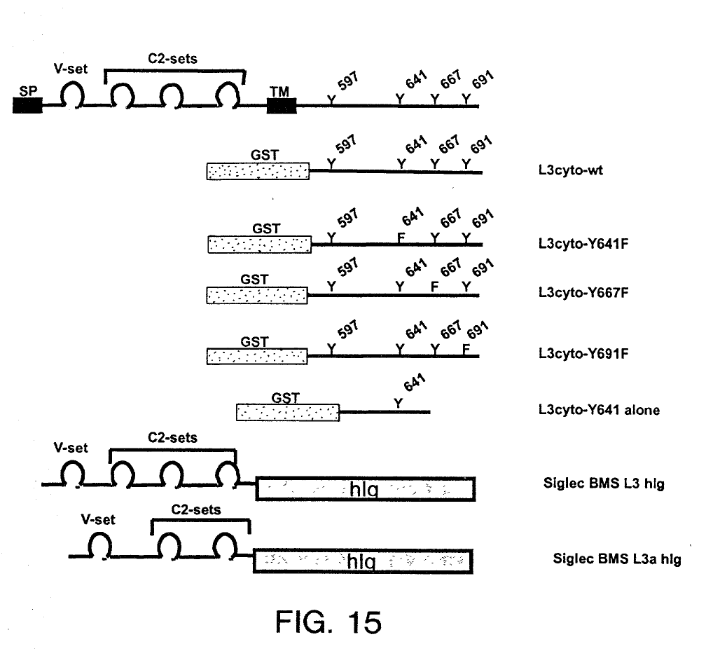

The mutant SIGLEC-BMS protein may include specific substitution of one or more amino acids within the cytoplasmic domain of a SIGLEC-BMS protein, e.g., mutation of a specific amino acid such as a tyrosine to a phenylalanine, leucine, tryptophan, or Thr (FIG. 15). Examples of mutant SIGLEC-BMS proteins include, but are not limited to SIGLEC-BMS proteins wherein at least one tyrosine at positions 597, 641, 667, or 691 is substituted with a phenylalanine as shown in FIG. 15, and described in Example 12.

Knowing which particular mutations in the cytoplasmic tail of a SIGLEC-BMS protein affect phosphorylation by various tyrosine kinases permits one skilled in the art to develop methods for screening ligands that affect SIGLEC-mediated cell signaling. For example, SIGLEC-mediated cell signalling can be mediated when tyrosine in any of positions 597, 641, 667, or 691, of FIG. 6b, is substituted with phenylalanine, leucine, tryptophan and threonine. Additionally, ligands that bind to the site so mutated within the cytoplasmic domain can be modified so as to modulate SIGLEC-mediated cell signalling, i.e., upregulating or downregulating cell signalling.

Methods For Generating SIGLEC-BMS Proteins

The SIGLEC-BMS proteins of the invention may be generated by recombinant methods. Recombinant methods are preferred if a high yield is desired. Recombinant methods involve expressing the cloned gene in a suitable host cell. For example, a host cell is introduced with an expression vector having a Siglec-BMS sequence, then the host cell is cultured under conditions that permit in vivo production of the SIGLEC-BMS protein encoded by the sequence.

For example, in general terms, the production of recombinant SIGLEC-BMS proteins can involve a host/vector system and the following steps. A nucleic acid molecule can be obtained that encodes a SIGLEC-BMS protein or a fragment thereof, such as any one of the polynucleotides disclosed in SEQ ID NOs.: 1-7, or 27. The SIGLEC-BMS-encoding nucleic acid molecule can be then preferably inserted into an expression vector in operable linkage with suitable expression control sequences, as described above, to generate an expression vector containing the SIGLEC-BMS-encoding sequence. The expression vector can be introduced into a suitable host, by standard transformation methods, and the resulting transformed host is cultured under conditions that allow the production and retrieval of the SIGLEC-BMS protein. For example, if expression of the SIGLEC-BMS gene is under the control of an inducible promoter, then suitable growth conditions include the appropriate inducer. The SIGLEC-BMS protein, so produced, is isolated from the growth medium or directly from the cells; recovery and purification of the protein may not be necessary in some instances where some impurities may be tolerated. A skilled artisan can readily adapt an appropriate host/expression system known in the art (Cohen, et al., supra; Maniatis et al., supra) for use with SIGLEC-BMS-encoding sequences to produce a SIGLEC-BMS protein.

The SIGLEC-BMS proteins of the invention, and fragments thereof, can be generated by chemical synthesis methods. The principles of solid phase chemical synthesis of polypeptides are well known in the art and may be found in general texts relating to this area (Dugas, H. and Penney, C. 1981 Bioorganic Chemistry, pp 54-92, Springer-Verlag, N.Y.). SIGLEC-BMS polypeptides may be synthesized by solid-phase methodology utilizing an Applied Biosystems 430A peptide synthesizer (Applied Biosystems, Foster City, Calif.) and synthesis cycles supplied by Applied Biosystems. Protected amino acids, such as t-butoxycarbonyl-protected amino acids, and other reagents are commercially available from many chemical supply houses.

The present invention provides derivative protein molecules, such as chemically modified proteins. Illustrative of such modifications would be replacement of hydrogen by an alkyl, acyl, or amino group. The SIGLEC-BMS protein derivatives retain the biological activities of natural SIGLEC-BMS proteins.

Recombinant Nucleic Acid Molecules Encoding SIGLEC-BMS

Also provided are recombinant DNA molecules (rDNAs) that include nucleotide sequences that encode SIGLEC-BMS proteins, or a fragment thereof, as described herein. As used herein, a rDNA molecule is a DNA molecule that has been subjected to molecular manipulation in vitro. Methods for generating rDNA molecules are well known in the art, for example, see Sambrook et al., Molecular Cloning (1989). In the preferred rDNA molecules of the present invention, the sequences that encode the SIGLEC-BMS proteins or fragments of SIGLEC, are operably linked to one or more expression control sequences and/or vector sequences.

Vectors

The nucleic acid molecules of the invention may be recombinant molecules each comprising the sequence, or portions thereof, of a Siglec-BMS sequence linked to a non-Siglec-BMS sequence. For example, the Siglec-BMS sequence may be fused operatively to a vector to generate a recombinant molecule.

The term vector includes, but is not limited to, plasmids, cosmids, and phagemids. A preferred vector will be an autonomously replicating vector comprising a replicon that directs the replication of the rDNA within the appropriate host cell. Alternatively, the preferred vector directs integration of the recombinant vector into the host cell. Various viral vectors may also be used, such as, for example, a number of well known retroviral and adenoviral vectors (Berkner 1988 Biotechniques 6:616-629).

The preferred vectors permit expression of the Siglec-BMS transcript or polypeptide sequences in prokaryotic or eukaryotic host cells. The preferred vectors include expression vectors, comprising an expression control element, such as a promoter sequence, which enables transcription of the inserted Siglec-BMS sequences and can be used for regulating the expression (e.g., transcription and/or translation) of an operably linked Siglec-BMS sequence in an appropriate host cell, such as Escherichia coli.

Expression control elements are known in the art and include, but are not limited to, inducible promoters, constitutive promoters, secretion signals, enhancers, transcription terminators, and other transcriptional regulatory elements. Other expression control elements that are involved in translation are known in the art, and include the Shine-Dalgarno sequence (e.g., prokaryotic host cells), and initiation and termination codons.

Specific initiation signals may also be required for efficient translation of a Siglec-BMS sequence. These signals include the ATG-initiation codon and adjacent sequences. In cases where the Siglec-BMS initiation codon and upstream sequences are inserted into the appropriate expression vector, no additional translational control signals may be needed. However, in cases where only the coding sequence, or a portion thereof, is inserted, exogenous transcriptional control signals including the ATG-initiation codon must be provided. Furthermore, the initiation codon must be in the correct reading-frame to ensure transcription of the entire insert. Exogenous transcriptional elements and initiation codons can be of various origins, both natural and synthetic. The efficiency of expression may be enhanced by the inclusion of enhancers appropriate to the cell system in use (Scharf, D., et al, 1994 Results Probl. Cell. Differ. 20:125-62; Bittner, et al., 1987 Methods in Enzymol. 153:516-544).

The preferred vectors for expression of the Siglec-BMS sequences in eukaryote host cells include expression control elements, such as the baculovirus polyhedrin promoter for expression in insect cells. Other expression control elements include promoters or enhancers derived from the genomes of plant cells (e.g., heat shock, RUBISCO, storage protein genes), viral promoters or leader sequences or from plant viruses, and promoters or enhancers from the mammalian genes or from mammalian viruses.

The preferred vector includes at least one selectable marker gene that encodes a gene product that confers drug resistance such as resistance to ampicillin or tetracyline. The vector also comprises multiple endonuclease restriction sites that enable convenient insertion of exogenous DNA sequences. Methods for generating a recombinant expression vector encoding the SIGLEC-BMS proteins of the invention are well known in the art, and can be found in Maniatis, T., et al., (1989 Molecular Cloning, A Laboratory Manual, Cold Spring Harbor Laboratory, Cold Spring Harbor, N.Y.) and Ausubel et al. (1989 Current Protocols in Molecular Biology, John Wiley & Sons, New York N.Y.).

The preferred vectors for generating Siglec-BMS transcripts and/or the encoded SIGLEC-BMS polypeptides are expression vectors which are compatible with prokaryotic host cells. Prokaryotic cell expression vectors are well known in the art and are available from several commercial sources. For example, pET vectors (e.g., pET-21, Novagen Corp.), BLUESCRIPT phagemid (Stratagene, LaJolla, Calif.), pSPORT (Gibco BRL, Rockville, Md.), or ptrp-lac hybrids may be used to express SIGLEC-BMS polypeptides in bacterial host cells.

Alternatively, the preferred expression vectors for generating Siglec-BMS transcripts and/or the encoded SIGLEC-BMS polypeptides are expression vectors which are compatible with eukaryotic host cells. The more preferred vectors are those compatible with vertebrate cells. Eukaryotic cell expression vectors are well known in the art and are available from several commercial sources. Typically, such vectors are provided containing convenient restriction sites for insertion of the desired DNA segment. Typical of such vectors are PSVL and pKSV-10 (Pharmacia), pBPV-1/pML2d (International Biotechnologies, Inc.), pTDT1 (ATCC, #31255), and similar eukaryotic expression vectors.

Host-Vector Systems

The invention further provides a host-vector system comprising a vector, plasmid, phagemid, or cosmid comprising a Siglec-BMS nucleotide sequence, or a fragment thereof, introduced into a suitable host cell. A variety of expression vector/host systems may be utilized to carry and express Siglec-BMS sequences. The host-vector system can be used to express (e.g., produce) the SIGLEC-BMS polypeptides encoded by Siglec-BMS nucleotide sequences. The host cell can be either prokaryotic or eukaryotic. Examples of suitable prokaryotic host cells include bacteria strains from genera such as Escherichia, Bacillus, Pseudomonas, Streptococcus, and Streptomyces. Examples of suitable eukaryotic host cells include yeast cells, plant cells, or animal cells such as mammalian cells. A preferred embodiment provides a host-vector system comprising the pcDNA3 vector (Invitrogen, Carlsbad, Calif.) in COS7 mammalian cells, pGEX vector (Promega, Madison, Wis.) in bacterial cells, or pFastBac vector (Gibco/BRL, Rockville, Md.) in Sf9 insect cells.

Introduction of the recombinant DNA molecules of the present invention into an appropriate host cell is accomplished by well known methods that depend on the type of vector used and host system employed. For example, prokaryotic host cells are introduced (e.g., transformed) with nucleic acid molecules by electroporation or salt treatment methods, see for example, Cohen et al., 1972 Proc Acad Sci USA 69:2110; Maniatis, T., et al., 1989 Molecular Cloning, A Laboratory Manual, Cold Spring Harbor Laboratory, Cold Spring Harbor, N.Y. Vertebrate cells are transformed with vectors containing recombinant DNAs by various methods, including electroporation, cationic lipid or salt treatment (Graham et al., 1973 Virol 52:456; Wigleretal., 1979 Proc Natl Acad Sci USA 76:1373-76).

Successfully transformed cells, i.e., cells that contain a rDNA molecule of the present invention, can be identified by techniques well known in the art. For example, cells resulting from the introduction of recombinant DNA of the present invention are selected and cloned to produce single colonies. Cells from those colonies are harvested, lysed and their DNA content examined for the presence of the rDNA using a method such as that described by Southern, J Mol Biol (1975) 98:503, or Berent et al., Biotech (1985) 3:208, or the proteins produced from the cell assayed via a biochemical assay or immunological method.

In bacterial systems, a number of expression vectors may be selected depending upon the use intended for the SIGLEC-BMS proteins. For example, when large quantities of SIGLEC-BMS proteins are needed for the induction of antibodies, vectors that direct high level expression of fusion proteins that are soluble and readily purified may be desirable. Such vectors include, but are not limited to, the multifunctional E. coli cloning and expression vectors such as BLUESCRIPT (Stratagene), in which the Siglec-BMS sequence may be ligated into the vector in-frame with sequences for the amino-terminal Met and the subsequent 7 residues of β-galactosidase so that a hybrid protein is produced; pIN vectors (Van Heeke & Schuster (1989) J Biol Chem 264:5503-5509); and the like. The pGEX vectors (Promega, Madison Wis.) may also be used to express foreign proteins as fusion proteins with glutathione S-transferase (GST). In general, such fusion proteins are soluble and can easily be purified from lysed cells by adsorption to glutathione-agarose beads followed by elution in the presence of free glutathione. Proteins made in such systems are designed to include heparin, thrombin or factor XA protease cleavage sites so that the cloned protein of interest can be released from the GST moiety at will.

In yeast, Saccharomyces cerevisiae, a number of vectors containing constitutive or inducible promoters such as beta-factor, alcohol oxidase and PGH may be used. For reviews, see Ausubel et al (supra) and Grant et al (1987) Methods in Enzymology 153:516-544.

In cases where plant expression vectors are used, the expression of a sequence encoding SIGLEC-BMS protein can be driven by any of a number of promoters. For example, viral promoters such as the 35S and 19S promoters of CaMV (Brisson, et al., (1984) Nature 310:511-514) may be used alone or in combination with the omega leader sequence from TMV (Takamatsu, et al., (1987) EMBO J 6:307-311). Alternatively, plant promoters such as the small subunit of RUBISCO (Coruzzi et al (1984) EMBO J 3:1671-1680; Broglie et al (1984) Science 224:838-843); or heat shock promoters (Winter J and Sinibaldi R M (1991) Results Probl Cell Differ 17:85-105) can be used. These constructs can be introduced into plant cells by direct DNA transformation or pathogen-mediated transfection. For reviews of such techniques, see Hobbs, S. in: McGraw Yearbook of Science and Technology (1992) McGraw Hill New York N.Y., pp 191-196; or Weissbach and Weissbach (1988) in: Methods for Plant Molecular Biology, Academic Press, New York N.Y., pp 421-463.

An alternative expression system that can be used to express SIGLEC-BMS proteins is an insect system. In one such system, Autographa califormica nuclear polyhedrosis virus (AcNPV) can be used as a vector to express foreign genes in Spodoptera frugiperda cells or in Trichoplusia larvae. The sequence encoding a SIGLEC-BMS protein can be cloned into a nonessential region of the virus, such as the polyhedrin gene, and placed under control of the polyhedrin promoter. Successful insertion of a Siglec-BMS nucleotide sequence will render the polyhedrin gene inactive and produce recombinant virus lacking coat protein. The recombinant viruses can then used to infect S. frugiperda cells or Trichoplusia larvae in which SIGLEC-BMS protein can be expressed (Smith et al (1983) J Virol 46:584; Engelhard E. K., et al, 1994 Proc NatAcadSci 91:3224-7).

In mammalian host cells, a number of viral-based expression systems can be utilized. In cases where an adenovirus is used as an expression vector, a Siglec-BMS sequence can be ligated into an adenovirus transcription/translation vector consisting of the late promoter and tripartite leader sequence. Insertion in a nonessential E1 or E3 region of the viral genome results in a viable virus capable of expressing a SIGLEC-BMS protein in infected host cells (Logan and Shenk 1984 Proc Natl Acad Sci 81:3655-59). In addition, transcription enhancers, such as the rous sarcoma virus (RSV) enhancer, can be used to increase expression in mammalian host cells.

In addition, a host cell strain may be chosen for its ability to modulate the expression of the inserted sequences or to process the expressed protein in the desired fashion. Such modifications of the protein include, but are not limited to, acetylation, carboxylation, glycosylation, phosphorylation, lipidation and acylation. Post-translational processing which cleaves a precursor form of the protein (e.g., a prepro protein) may also be important for correct insertion, folding and/or function. Different host cells such as CHO, HeLa, MDCK, 293, W138, etc. have specific cellular machinery and characteristic mechanisms for such post-translational activities and may be chosen to ensure the correct modification and processing of the introduced, foreign protein.

For long-term, high-yield production of recombinant proteins, stable expression is preferred. For example, cell lines that stably express SIGLEC-BMS proteins can be transformed using expression vectors that contain viral origins of replication or endogenous expression elements and a selectable marker gene. Following the introduction of the vector, cells can be grown in an enriched media before they are switched to selective media. The purpose of the selectable marker is to confer resistance to selection, and its presence allows growth and recovery of cells which successfully express the introduced sequences. Resistant clumps of stably transformed cells can be proliferated using tissue culture techniques appropriate for the cell type used.

Any number of selection systems may be used to recover transformed cell lines. These include, but are not limited to, the herpes simplex virus thymidine kinase (Wigler, M., et al., 1977 Cell 11:223-32) and adenine phosphoribosyltransferase (Lowy, I. et al., 1980 Cell 22:817-23) genes which can be employed in tk-minus or aprt-minus cells, respectively. Also, antimetabolite, antibiotic or herbicide resistance can be used as the basis for selection; for example, dhfr which confers resistance to methotrexate (Wigler, M., et al., 1980 Proc Natl Acad Sci 77:3567-70); npt, which confers resistance to the aminoglycosides neomycin and G-418 (Colbere-Garapin, F., et al., 1981 J. Mol. Biol. 150:1-14) and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman, S. C. and R. C. Mulligan 1988 Proc. Natl. Acad. Sci. 85:8047-51). Recently, the use of visible markers has gained popularity with such markers as anthocyanins, β-glucuronidase and its substrate, GUS, and luciferase and its substrate, luciferin, being widely used not only to identify transformants, but also to quantify the amount of transient or stable protein expression attributable to a specific vector system (Rhodes, C. A., et al., 1995 Methods Mol. Biol. 55:121-131).

Antibodies Reactive Against SIGLEC-BMS Proteins and Polypeptides

The invention further provides antibodies, such as polyclonal, monoclonal, chimeric, fragments, and humanized antibodies, that bind to SIGLEC-BMS proteins or fragments of SIGLEC-BMS proteins thereof. Particular examples of monoclonal antibodies of the invention are those designated Siglec-10-9, Siglec-10-13, Siglec-10-14, Siglec-10-27, and Siglec-10-61, which collectively were deposited on Jul. 18, 2001 with the American Type Culture Collection (ATCC), 10801 University Blvd., Manassas, Va. 20110-2209 under the provisions of the Budapest Treaty, and accorded ATCC accession number (______). These antibodies can be easily separated from the collective deposit by standard separation techniques such as subcloning or isotype separation (Harlow, E. and Lane, D. 1988 Antibodies, A Laboratory Manual, Cold Spring Harbor Laboratory, Cold Spring Harbor, N.Y.).

Mabs Siglec-10-9, Siglec-10-13, Siglec-10-14, Siglec-10-27, and Siglec-10-61 all recognize and bind Siglec-10 and display different or similar isotypes. For example, the isotype of Siglec-10-9 is IgG3 kappa isotype, Siglec-10-13 is IgG2b kappa isotype, Siglec-10-14 is IgG1 kappa isotype, Siglec-10-27 is IgG1 kappa isotype, and Siglec-10-61 is IgG2a kappa isotype.

Preferably, the antibodies of the invention bind specifically to polypeptides having SIGLEC-BMS sequences. For example, the antibodies of the invention can recognize and bind to a SIGLEC-BMS protein comprising an amino acid sequence beginning with Ala141 and ending with Ser198 as shown in FIG. 6B (SEQ ID NO:28). In another embodiment, the antibody of the invention can recognizes and binds a SIGLEC-BMS protein comprising an amino acid sequence beginning with Ala141 and ending with Ser198 as shown in FIG. 6B (SEQ ID NO:28) and is encoded by a nucleic acid molecule that hybridizes, under stringent conditions to a nucleic acid molecule that is complementary to the nucleic acid as shown in any one of SEQ. ID NOS. 1-7 and 27. Additionally, the antibody of the invention can recognize and bind a SIGLEC protein comprising an amino acid sequence that is encoded by a nucleic acid molecule that hybridizes, under stringent conditions to a nucleic acid molecule that is complementary to the nucleic acid as shown in any one of SEQ ID NOS. 1-7 and 27. Preferably, the antibody of the invention can recognize and bind SIGLEC-BMS-L3a, -L3b, -L3c, -L3d, -L4a, -L5a, -L5b, and -L3-995-2 proteins (FIGS. 2B, 3B, 4B, 5B, 7B, 8B, 9B, and 6B).

Most preferably, a SIGLEC-BMS antibody specifically bind to the extracellular domain of a SIGLEC-BMS protein. The extracellular domain can be any or all of the Ig-like domains of Siglec-10. Specifically, the antibody can recognize and bind the second Ig-like (Ig-D2) domain (Ala14l-Ser198) or the Ig-D5 domain (Tyr444-Pro538) as shown in FIG. 25. In other embodiments, the antibodies of the invention specifically bind to other domains of a SIGLEC-BMS protein or precursor, for example the antibodies bind to the cytoplasmic domain of SIGLEC-BMS proteins. For example, the cytoplasmic domain can encompass amino acids Lys576 through Gln697 as shown in FIG. 6B.

The most preferred antibodies will selectively bind to SIGLEC-BMS proteins and will not bind (or will bind weakly) to non-SIGLEC-BMS proteins. These antibodies can be from any source, e.g., rabbit, sheep, rat, dog, cat, pig, horse, mouse and human.

As will be understood by those skilled in the art, the regions or epitopes of a SIGLEC-BMS protein to which an antibody is directed may vary with the intended application. For example, antibodies intended for use in an immunoassay for the detection of membrane-bound SIGLEC-BMS on viable cells should be directed to an accessible epitope such as the extracellular domain of SIGLEC-BMS proteins. Anti-SIGLEC-BMS mAbs can be used to stain the cell surface of SIGLEC-BMS-positive cells. The predicted extracellular domain of SIGLEC-BMS proteins represent potential markers for screening, diagnosis, prognosis, and follow-up assays and imaging methods. In addition, SIGLEC-BMS proteins may be excellent targets for therapeutic methods such as targeted antibody therapy, immunotherapy, and gene therapy to treat conditions associated with the presence or absence of SIGLEC-BMS proteins. Antibodies that recognize other epitopes may be useful for the identification of SIGLEC-BMS within damaged or dying cells, for the detection of secreted SIGLEC-BMS proteins or fragments thereof. Additionally, some of the antibodies of the invention may be internalizing antibodies, which internalize (e.g., enter) into the cell upon or after binding. Internalizing antibodies are useful for inhibiting cell growth and/or inducing cell death.

The invention includes any monoclonal antibody, the antigen-binding region of which competitively inhibits the immunospecific binding of any of the monoclonal antibodies of the invention to its target antigen. These monoclonal antibodies may be identified by routine competition assays using, for example, any of the antibodies Siglec-10-9, Siglec-10-13, Sigle10-14, Siglec-10-27, and Siglec-10-61 (Harlow, E. and Lane, D. 1988 Antibodies, A Laboratory Manual, Cold Spring Harbor Laboratory, Cold Spring Harbor, N.Y.). Further, the invention provides recombinant proteins comprising the antigen-binding region of any the monoclonal antibodies of the invention.

The invention also encompasses antibody fragments that specifically recognize a SIGLEC-BMS protein or a fragment thereof. As used herein, an antibody fragment is defined as at least a portion of the variable region of the immunoglobulin molecule that binds to its target, i.e., the antigen binding region. Some of the constant region of the immunoglobulin may be included. Fragments of the monoclonal antibodies or the polyclonal antisera include Fab, F(ab′)₂, Fv fragments, single-chain antibodies, and fusion proteins which include the immunologically significant portion (i.e., a portion that recognizes and binds SIGLEC-BMS).

The chimeric antibodies of the invention are immunoglobulin molecules that comprise at least two antibody portions from different species, for example a human and non-human portion. Chimeric antibodies are useful, as they are less likely to be antigenic to a human subject than antibodies with non-human constant regions and variable regions. The antigen combining region (variable region) of a chimeric antibody can be derived from a non-human source (e.g. murine) and the constant region of the chimeric antibody, which confers biological effector function to the immunoglobulin, can be derived from a human source (Morrison et al., 1985 Proc. Natl. Acad. Sci. U.S.A. 81:6851; Takeda et al., 1985 Nature 314:452; Cabilly et al., U.S. Pat. No. 4,816,567; Boss et al., U.S. Pat. No. 4,816,397). The chimeric antibody may have the antigen binding specificity of the non-human antibody molecule and the effector function conferred by the human antibody molecule.

The chimeric antibodies of the present invention also comprise antibodies which are chimeric proteins, having several distinct antigen binding specificities (e.g. anti-TNP: Boulianne et al., 1984 Nature 312:643; and anti-tumor antigens: Sahagan et al., 1986 J. Immunol. 137:1066). The invention also provides chimeric proteins having different effector functions (Neuberger et al., 1984 Nature 312:604), immunoglobulin constant regions from another species and constant regions of another immunoglobulin chain (Sharon et al., 1984 Nature 309:364); Tan et al., 1985 J. Immunol. 135:3565-3567). Additional procedures for modifying antibody molecules and for producing chimeric antibody molecules using homologous recombination to target gene modification have been described (Fell et al., 1989 Proc. Natl. Acad. Sci. USA 86:8507-8511).

Humanized antibodies directed against SIGLEC-BMS proteins are also useful. As used herein, a humanized SIGLEC-BMS antibody is an immunoglobulin molecule which is capable of binding to a SIGLEC-BMS protein. A humanized SIGLEC-BMS antibody includes variable regions having substantially the amino acid sequence of a human immunoglobulin and the hyper-variable region having substantially the amino acid sequence of non-human immunoglobulin. Humanized antibodies can be made according to several methods known in the art (Teng et al., 1983 Proc. Natl. Acad. Sci. U.S.A. 80:7308-7312; Kozbor et al., 1983 Immunology Today 4:7279; Olsson et al., 1982 Meth. Enzymol. 92:3-16).

Various methods for the preparation of antibodies are well known in the art. For example, antibodies may be prepared by immunizing a suitable mammalian host with an immunogen such as an isolated SIGLEC-BMS protein, peptide, fragment, or an immunoconjugated form of SIGLEC-BMS protein (Harlow 1989, in: Antibodies, Cold Spring Harbor Press, N.Y.). In addition, fusion proteins of SIGLEC-BMS may also be used as immunogens, such as a SIGLEC-BMS fused to -GST-, -human Ig, or His-tagged fusion proteins. Cells expressing or overexpressing SIGLEC-BMS proteins may also be used for immunizations. Similarly, any cell engineered to express SIGLEC-BMS proteins may be used. This strategy may result in the production of monoclonal antibodies with enhanced capacities for recognizing endogenous SIGLEC-BMS proteins (Harlow and Lane, 1988, in: Antibodies: A Laboratory Manual. Cold Spring Harbor Press).

The amino acid sequence of SIGLEC-BMS proteins, and fragments thereof, may be used to select specific regions of the SIGLEC-BMS proteins for generating antibodies. For example, hydrophobicity and hydrophilicity analyses of the SIGLEC-BMS amino acid sequence may be used to identify hydrophilic regions in the SIGLEC-BMS protein structure. Regions of the SIGLEC-BMS protein that show immunogenic structure, as well as other regions and domains, can readily be identified using various other methods known in the art (Rost, B., and Sander, C. 1994 Protein 19:55-72), such as Chou-Fasman, Garnier-Robson, Kyte-Doolittle, Eisenberg, Karplus-Schultz or Jameson-Wolf analysis. Fragments including these residues are particularly suited in generating anti-SIGLEC-BMS antibodies.

Methods for preparing a protein for use as an immunogen and for preparing immunogenic conjugates of a protein with a carrier such as BSA, KLH, or other carrier proteins are well known in the art. Techniques for conjugating or joining therapeutic agents to antibodies are well known (Arnon et al., “Monoclonal Antibodies For Immunotargeting Of Drugs In Cancer Therapy”, in: Monoclonal Antibodies And Cancer Therapy, Reisfeld et al. (eds.), pp. 243-56 (Alan R. Liss, Inc. 1985); Hellstrom et al., “Antibodies For Drug Delivery”, in: Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in: Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immune. Rev., 62:119-58 (1982); Sodee et al., 1997, Clin. Nuc. Med. 21: 759-766). In some circumstances, direct conjugation using, for example, carbodiimide reagents may be used; in other instances linking reagents such as those supplied by Pierce Chemical Co., Rockford, Ill., may be effective.

Administration of a SIGLEC-BMS immunogen is conducted generally by injection over a suitable time period and with use of a suitable adjuvant, as is generally understood in the art. During the immunization schedule, titers of antibodies can be taken to determine adequacy of antibody formation.

While the polyclonal antisera produced in this way may be satisfactory for some applications, for pharmaceutical compositions, monoclonal antibody preparations are preferred. Immortalized cell lines which secrete a desired monoclonal antibody may be prepared using the standard method of Kohler and Milstein (Nature 256: 495-497) or modifications which effect immortalization of lymphocytes or spleen cells, as is generally known. The immortalized cell lines secreting the desired antibodies are screened by immunoassay in which the antigen is the SIGLEC-BMS protein or a fragment thereof. When the appropriate immortalized cell culture secreting the desired antibody is identified, the cells can be cultured either in vitro or by production in ascites fluid. The desired monoclonal antibodies are then recovered from the culture supernatant or from the ascites supernatant.

Novel antibodies of human origin can be also made to the antigen having the appropriate biological functions. The completely human antibodies are particularly desirable for therapeutic treatment of human patients. The human monoclonal antibodies may be made by using the antigen, e.g. a SIGLEC-BMS protein or peptide thereof, to sensitize human lymphocytes to the antigen in vitro, followed by EBV-transformation or hybridization of the antigen-sensitized lymphocytes with mouse or human lymphocytes, as described by Borrebaeck et al. (Proc. Natl. Acad. Sci. USA 85:3995-99 (1988)).

Alternatively, human antibodies can be produced using transgenic animals such as mice which are incapable of expressing endogenous immunoglobulin heavy and light chain genes, but which can express human heavy and light chain genes. The transgenic mice are immunized in the normal fashion with a selected antigen, e.g., all or a portion of a polypeptide of invention. Monoclonal antibodies directed against the antigen can be produced using conventional hybridoma technology. The human immunoglobulin transgenes harbored by the transgenic mice rearrange during B cell differentiation, and subsequently undergo class switching and somatic mutations. Thus, using this technology, it is possible to produce therapeutically useful IgG, IgA, and IgB antibodies. For an overview of this technology to produce human antibodies, see Lonberg and Haszar (1995, Int. Rev. Immunol. 13;65-93). A detailed discussion of this technology for producing human antibodies and human monoclonal antibodies can be found in U.S. Pat. Nos. 5,625,126; 5,633,425; 5,569,825; 5,661,016; and 5,545,806.

The antibodies or fragments may also be produced by recombinant means. The antibody regions that bind specifically to the desired regions of the SIGLEC-BMS protein can also be produced in the context of chimeric or CDR grafted antibodies of multiple species origin.

Uses of the Molecules of the Invention

The nucleic acid molecules encoding SIGLEC-BMS proteins are useful for a variety of purposes, including their use in diagnosis and/or prognosis methods. The nucleic acid molecules and proteins of the invention may be used to test the presence and/or amount of Siglec-BMS nucleotide sequences and/or SIGLEC-BMS protein in a suitable biological sample.

The suitable biological sample can be from an animal or a human. The sample can be a cell sample or a tissue sample, including samples from spleen, lymph node, thymus, bone marrow, liver, heart, brain, placenta, lung, skeletal muscle, kidney and pancreas. The sample can be a biological fluid, including, urine, blood sera, blood plasma, phlegm, or lavage fluid. Alternatively, the sample can be a swab from the nose, ear or throat.

Additionally, the SIGLEC-BMS proteins are able to elicit the generation of antibodies, which can serve as molecules for use in various diagnostic or therapeutic modalities. SIGLEC-BMS proteins may also be used to identify and isolate agents that bind to SIGLEC-BMS proteins (e.g., SIGLEC-BMS ligands) and modulate the biological activity of SIGLEC-BMS proteins.

Uses of Nucleic Acid Molecules Encoding Siglec-BMS Proteins

The nucleic acid molecules encoding SIGLEC-BMS proteins can be used in various hybridization methods to identify and/or isolate nucleotide sequences related to the Siglec-BMS nucleotide sequence described herein. Sequences related to Siglec-BMS sequence are useful for developing additional ligands and antibodies. The hybridization methods are used to identify/isolate DNA and RNA sequences that are identical or similar to the Siglec-BMS sequences, such as SIGLEC-BMS homologues, alternatively sliced isoforms, allelic variants, and mutant forms of the SIGLEC protein, as well as their coding and gene sequences.

Full-length or fragments of the nucleotide sequences that encode the SIGLEC-BMS proteins, described herein, can be used as a nucleic acid probes to retrieve nucleic acid molecules having sequences related to Siglec-BMS sequences.

In one embodiment, a Siglec-BMS nucleic acid probe is used to screen genomic libraries, such as libraries constructed in lambda phage or BACs (bacterial artificial chromosomes) or YACs (yeast artificial chromosomes), to isolate a genomic clone of a Siglec gene. Siglec-BMS sequences from genomic libraries are useful for isolating upstream or downstream non-coding sequences, such as promoter, enhancer, and transcription termination sequences. The upstream sequences may be joined to non-Siglec-BMS sequences in order to construct a recombinant DNA molecule that expresses the non-Siglec-BMS sequence upon introduction into an appropriate host cell. In another embodiment, a Siglec-BMS probe is used to screen cDNA libraries to isolate cDNA clones expressed in certain tissues or cell types. Siglec-BMS sequences from cDNA libraries are useful for isolating sequences from various cell types, tissue types, or from various mammalian subjects.

Additionally, pairs of oligonucleotide primers can be prepared for use in a polymerase chain reaction (PCR) to selectively amplify or clone nucleic acid molecules encoding SIGLEC-BMS proteins, or fragments thereof. PCR methods (U.S. Pat. No. 4,965,188) that include numerous cycles of denature/anneal/polymerize steps are well known in the art and can readily be adapted for use in isolating other SIGLEC-BMS-encoding nucleic acid molecules.

In addition, the nucleic acid molecules of the invention may also be employed in diagnostic embodiments, using the Siglec-BMS nucleic acid probes to determine the presence and/or the amount of Siglec-BMS sequences present in a biological sample.

One diagnostic embodiment encompasses determining the amount of Siglec-BMS nucleotide sequences present within asuitable biological sample, using a Siglec-BMS probe in a hybridization procedure.

Another embodiment encompasses quantifying the amount of Siglec-BMS nucleic acid molecules in the biological sample from a test subject, using a Siglec-BMS probe in a hybridization procedure. The amount of Siglec-BMS nucleic acid molecules in the test sample can be compared with the amount of Siglec-BMS nucleic acid molecules in a reference sample from a normal subject. The presence of a measurably different amount of Siglec-BMS nucleic acid molecules between the test and reference samples may correlate with the presence or with the severity of a disease associated with abnormal levels or a deficiency of Siglec-BMS nucleic acid molecules.

In another embodiment, monitoring the amount of Siglec-BMS RNA transcripts over time is effected by quantitatively determining the amount of Siglec-BMS RNA transcripts in test samples taken at different points in time. A difference in the amounts of Siglec-BMS RNA transcripts in the various samples being indicative of the course of a disease associated with expression of a Siglec-BMS transcripts.

As a further embodiment, the diseases or disorders associated with Siglec-BMS transcripts or proteins are detected by an increase or deficiency in Siglec-BMS gene copy number. Methods for detecting gene copy number include chromosome mapping by Fluorescence In Situ Hybridization (FISH analysis) (Rowley et al., 1990, PNAS USA 87: 9358-9362, H. Shizuya, PNAS USA, 89:8794). Methods for determining an increase in Siglec-BMS gene copy number are important because the increase may correlate with an increase in the severity of the disease associated with SIGLEC-BMS protein and poor patient outcome.

To conduct such diagnostic methods, a suitable biological sample from a test subject is contacted with a Siglec-BMS probe, under conditions effective to allow hybridization between the sample nucleic acid molecules and the probe. In a similar manner, a biological sample from a normal subject is contacted with a Siglec-BMS probe and hybridized under similar conditions. The presence of the nucleic acid molecules hybridized to the probe is detected. The relative and/or quantified amount of the hybridized molecules may be compared between the test and reference samples. The Siglec-BMS probes are preferably labeled with any of the known detectable labels, including radioactive, enzymatic, fluorescent, or even chemiluminescent labels.

Many suitable variations of hybridization technology are available for use in the detection of nucleic acids having Siglec-BMS sequences. These include, for example, Southern and Northern procedures. Other hybridization techniques and systems are known that can be used in connection with the detection aspects of the invention, including diagnostic assays such as those described in Falkow et al., U.S. Pat. No. 4,358,535. Another hybridization procedure includes in situ hybridization, where the target nucleic acids are located within one or more cells and are contacted with the Siglec-BMS probes. As is well known in the art, the cells are prepared for hybridization by fixation, e.g. chemical fixation, and placed in conditions that permit hybridization of the Siglec-BMS probe with nucleic acids located within the fixed cell.

Alternatively, Siglec-BMS nucleic acids are separated from a test sample prior to contact with a probe. The methods for isolating target nucleic acids from the sample are well known, and include cesium chloride gradient centrifugation, chromatography (e.g., ion, affinity, magnetic), and phenol extraction.

Uses of SIGLEC-BMS Proteins

SIGLEC-BMS proteins are expressed in eosinophils, neutrophils and monocytes and the expression of these molecules is immune-restricted, indicating that these proteins may be involved in modulating eosinophil or other immune cell maturation, migration, activation, or communication with other cells. Thus, SIGLEC-BMS proteins are postulated to be involved in the pathogenesis of asthma and other allergic diseases, leukemia, or inflammation.

SIGLEC-BMS proteins are thus attractive targets for drug development. Drugs directed against SIGLEC-BMS will likely inhibit inflammation, tissue damage and remodeling in asthma and possibly other inflammatory diseases such as allergic rhinitis, osteoarthritis, inflammatory bowel disease, Crohn's disease, chronic obstructive pulmonary disease, psoriasis, conjunctivitis, glomerular nephritis, rheumatoid arthritis and gingivitis. In addition, given that previously discovered SIGLEC proteins have been detected on circulating, immature white blood cells in some types of monomyelocytic leukemias (Elghetany, M. T. 1998 Haematologica 83:1104-1115), it is likely that drugs directed against SIGLEC-BMS proteins could be used to treat or target certain types of leukemia (e.g., eosinophilic leukemia).

In addition, the SIGLEC-BMS proteins and fragments of the invention can be used to elicit the generation of antibodies that specifically bind an epitope associated with SIGLEC-BMS protein, as described herein (Kohler and Milstein, supra). The SIGLEC-BMS antibodies include fragments, such Fv, Fab′, and F(ab′)2. SIGLEC-BMS antibodies which are immunoreactive with selected domains or regions of the SIGLEC-BMS protein are particularly useful. The domains of interest include the extracellular and cytoplasmic domains of SIGLEC-BMS proteins.

In one embodiment, the SIGLEC-BMS antibodies are used to screen expression libraries in order to obtain proteins related to SIGLEC-BMS proteins (e.g., homologues).

In another embodiment, SIGLEC-BMS antibodies are used to enrich or purify SIGLEC-BMS proteins from a sample having a heterologous population of proteins. The enrichment and purifying methods include conventional techniques, such as immuno-affinity methods. In general, the immuno-affinity methods include the following steps: preparing an affinity matrix by linking a solid support matrix with SIGLEC-BMS antibodies, which linked affinity matrix specifically binds with SIGLEC-BMS proteins; contacting the linked affinity matrix with the sample under conditions that permit the SIGLEC-BMS proteins in the sample to bind to the linked affinity matrix; removing the non-SIGLEC-BMS proteins that did not bind to the linked affinity matrix, thereby enriching or purifying for the SIGLEC-BMS proteins. A further step may include eluting the SIGELC-BMS proteins from the affinity matrix. The general steps and conditions for affinity enrichment for a desired protein or protein complex can be found in Antibodies: A Laboratory Manual (Harlow, E. and Lane, D., 1988 CSHL, Cold Spring, N.Y.).

SIGLEC-BMS antibodies are also used to detect, sort, or isolate cells expressing a SIGLEC-BMS protein. The SIGLEC-BMS-positive (+) cells are detected within various biological samples. The presence of SIGLEC-BMS proteins on cells (alone or in combination with other cell surface markers) may be used to distinguish and isolate cells (e.g., sorting) expressing SIGLEC-BMS from other cells, using antibody-based cell sorting or affinity purification techniques. The SIGLEC-BMS antibodies may be used to generate large quantities of relatively pure SIGLEC-BMS-positive cells from individual subjects or patients, which can be grown in tissue culture. In this way, for example, an individual subject's cells may be expanded from a limited biopsy sample and then tested for the presence of diagnostic and prognostic genes, proteins, chromosomal aberrations, gene expression profiles, or other relevant genotypic and phenotypic characteristics, without the potentially confounding variable of contaminating cells. Similarly, patient-specific vaccines and cellular immunotherapeutics may be created from such cell preparations. The methods for detecting, sorting, and isolating SIGLEC-BMS-positive cells use various imaging methodologies, such as fluorescence or immunoscintigraphy with Induim-111 (or other isotope).

There are multiple diagnostic uses of the antibodies of the invention. For example, CD33 is upregulated in myelodysplastic syndromes (Elghetamy, 1998 supra) and is used as a diagnostic marker for leukemia. The invention provides methods for diagnosing in a subject, e.g., an animal or human subject, a disease associated with the presence or deficiency of the SIGLEC-BMS protein(s). In one embodiment, the method comprises quantitatively determining the amount of SIGLEC-BMS protein in the sample (e.g., cell or biological fluid sample) using any one or combination of the antibodies of the invention. Then the amount so determined can be compared with the amount in a sample from a normal subject. The presence of a measurably different amount in the sample (i.e., the amount of SIGLEC-BMS proteins in the test sample exceeds or is reduced from the amount of SIGLEC-BMS proteins in a normal sample) indicates the presence of the disease.

The anti-SIGLEC-BMS antibodies of the invention may be particularly useful in diagnostic imaging methodologies, where the antibodies have a detectable label. In accordance with the practice of the invention, the methods could use any monoclonal antibody that recognizes a SIGLEC BMS protein or fragment thereof including those antibodies, the antigen-binding region of which, competitively inhibits the immunospecific binding of any of the monoclonal antibodies Siglec 10-9, Siglec 10-13, Siglec 10-14, Siglec 10-27, or Siglec 10-61, to its target antigen.

The invention provides various immunological assays useful for the detection of SIGLEC-BMS proteins in a suitable biological sample. Suitable detectable markers include, but are not limited to, a radioisotope, a fluorescent compound, a bioluminescent compound, chemiluminescent compound, a chromophore, a metal chelator, biotin, or an enzyme. Such assays generally comprise one or more labeled SIGLEC-BMS antibodies that recognize and bind a SIGLEC-BMS protein, and include various immunological assay formats well known in the art, including but not limited to various types of precipitation, agglutination, complement fixation, radioimmunoassays (RIA), enzyme-linked immunosorbent assays (ELISA), enzyme-linked immunofluorescent assays (ELIFA) (H. Liu et al. 1998 Cancer Research 58: 4055-4060), immunohistochemical analyses and the like.

In addition, immunological imaging methods that detect cells expressing SIGLEC-BMS are also provided by the invention, including but not limited to radioscintigraphic imaging methods using labeled SIGLEC-BMS antibodies. Such assays may be clinically useful in the detection and monitoring the number and/or location of cells expressing SIGLEC-BMS proteins.

The invention additionally provides methods of determining a difference in the amount and distribution of SIGLEC-BMS protein in a test biological sample from an afflicted subject relative to the amount and distribution in a reference sample from a normal subject. In one embodiment, the method comprises contacting the test and reference sample with an anti-SIGLEC-BMS antibody that specifically forms a complex with a SIGLEC-BMS protein, thereby providing a means for detecting the difference in the amount and distribution of SIGLEC-BMS in the test and reference samples.

Additionally, the invention provides methods for monitoring the course of disease or disorders associated with SIGLEC-BMS in a test subject by measuring the amount of SIGLEC-BMS protein in a sample from the test subject at various points in time. This is done for purposes of determining a change in the amount of SIGLEC-BMS in the sample over time. Monitoring the course of disease or disorders may optimize the timing, dosage, and type of treatment, over time. In one embodiment, the method comprises quantitatively determining in a first sample from the subject the presence of a SIGLEC-BMS protein and comparing the amount so determined with the amount present in a second sample from the same subject taken at a different point in time, a difference in the amounts determined being indicative of the course of the disease.

One embodiment of the invention is a method for diagnosing an asthmatic condition in a candidate subject. This method comprises: obtaining a biological sample from an candidate asthmatic subject (e.g., test sample) and from normal subjects (e.g., reference samples); contacting the test and reference sample(s) with an anti-SIGLEC-BMS antibody that specifically forms a complex with a SIGLEC-BMS protein; detecting the complex so formed in the test and reference samples; comparing the amount of complex formed in the test and reference samples, where a measurable difference in the amount of the complex formed in the test and reference samples is indicative of an asthmatic condition. Elevated levels of SIGLEC-BMS in the bloodstream or lavage fluid may be a way of detecting the condition or severity of asthma. This detection can be done by ELISA or similar methods using antibodies that react with SIGLEC-BMS proteins.

SIGLEC-BMS antibodies may also be used therapeutically to modulate (e.g., inhibit or activate) the biological activity of SIGLEC-BMS proteins, or to target therapeutic agents, such as anti-inflammatory drugs, to cells expressing SIGLEC-BMS proteins. For example, cells expressing SIGLEC-BMS can be targeted, using antibodies that bind with cells expressing SIGLEC-BMS proteins. The binding of the SIGLEC-BMS antibody with the cells decrease the biological activity of SIGLEC-BMS proteins, thereby inhibiting the growth of the SIGLEC-BMS-expressing cell and decreasing the disease associated with abnormal cellular expression of SIGLEC-BMS proteins.

The SIGLEC-BMS antibodies or fragments thereof may be conjugated to a second molecule, such as a therapeutic agent (e.g., a cytotoxic agent) resulting in an immunoconjugate. The immunoconjugate can be used for targeting the second molecule to a SIGLEC-BMS positive cell, thereby inhibiting the growth of the SIGLEC-BMS positive cell (Vitetta, E. S. et al., 1993 “Immunotoxin Therapy” pp. 2624-2636, in: Cancer: Principles and Practice of Oncology, 4th ed., ed.: DeVita, Jr., V. T. et al., J.B. Lippincott Co., Philadelphia).

The therapeutic agents include, but are not limited to, anti-tumor drugs, cytotoxins, radioactive agents, cytokines, and a second antibody or an enzyme. Examples of cytotoxic agents include, but are not limited to ricin, doxorubicin, daunorubicin, taxol, ethiduim bromide, mitomycin, etoposide, tenoposide, vincristine, vinblastine, colchicine, dihydroxy anthracin dione, actinomycin D, diphteria toxin, Pseudomonas exotoxin (PE) A, PE40, abrin, and glucocorticoid and other chemotherapeutic agents, as well as radioisotopes.

Further, the invention provides an embodiment wherein the antibody of the invention is linked to an enzyme that converts a prodrug into a cytotoxic drug. Alternatively, the antibody is linked to enzymes, lymphokines, or oncostatin.

Use of immunologically reactive fragments, such as Fab, Fab′, or F(ab′)₂ fragments is often preferable, especially in a therapeutic context, as these fragments are generally less immunogenic than the whole immunoglobulin. The invention also provides pharmaceutical compositions having the monoclonal antibodies or anti-idiotypic monoclonal antibodies of the invention, in a pharmaceutically acceptable carrier.

Screening For SIGLEC-BMS Ligands

Another aspect of the invention relates to screening methods for identifying agents of interest that bind with (e.g., ligands) and/or modulate the biological activity of SIGLEC-BMS proteins. Because SIGLEC-BMS proteins are expressed in eosinophils, these agents may be involved in modulating eosinophil or other immune cell maturation, migration, activation, or communication with other cells. Thus, agents that bind with and modulate the biological activity of SIGLEC-BMS proteins may be effective in reducing certain symptoms of asthma and other allergic diseases, leukemia, or reduce inflammation.

Typically, the goal of such screening methods is to identify an agent(s) that binds to the target polypeptide (e.g., SIGLEC-BMS) and causes a change in the biological activity of the target polypeptide, such as activation or inhibition of the target polypeptide, thereby decreasing diseases associated with abnormal cellular expression of SIGLEC-BMS proteins. The agents of interest are identified from a population of candidate agents.

The screening methods include assays for detecting and identifying agents, and cellular constituents that bind to SIGLEC-BMS proteins (e.g., ligands of SIGLEC-BMS). In one embodiment, a screening assay comprises the following: contacting a SIGLEC-BMS protein with a test agent or cellular extract, under conditions that allow association (e.g., binding) of the SIGLEC-BMS protein with the test agent or a component of the cellular extract; and determining if a complex comprising the agent or component associated with the SIGLEC-BMS protein is formed. The screening methods are suitable for use in high through-put screening methods

The binding of an agent with a SIGLEC-BMS protein can be assayed using a shift in the molecular weight or a change in biological activity of the unbound SIGLEC-BMS protein, or the expression of a reporter gene in a two-hybrid system (Fields, S. and Song, O., 1989, Nature 340:245-246). The method used to identify whether an agent/cellular component binds to a SIGLEC-BMS protein is based primarily on the nature of the SIGLEC-BMS protein used. For example, a gel retardation assay is used to determine whether an agent binds to SIGLEC-BMS or a fragment thereof. Alternatively, immunodetection and biochip (e.g., U.S. Pat. No. 4,777,019) technologies are adopted for use with the SIGLEC-BMS protein. An alternative method for identifying agents that bind with SIGLEC-BMS proteins employs TLC overlay assays using glycolipid extracts from immune-type cells (K. M. Abdullah, et al., 1992 Infect. Immunol. 60:56-62). A skilled artisan can readily employ numerous art-known techniques for determining whether a particular agent binds to a SIGLEC-BMS protein.

Alternatively or consecutively, the biological activity of the SIGLEC-BMS protein, as part of the complex, can be analyzed as a means for identifying agonists and antagonists of SIGLEC-BMS activity. For example, a method used to isolate cellular components that bind CD22 (D. Sgroi, et al., 1993 J. Biol. Chem. 268:7011-7018; L. D. Powell, et al., 1993 J. Biol. Chem. 268:7019-7027) is adapted to isolate cell-surface glycoproteins that bind to SIGLEC-BMS proteins by contacting cell extracts with an affinity column having immobilized anti-SIGLEC-BMS antibodies.

As used herein, an agent is said to antagonize SIGLEC-BMS activity when the agent reduces the biological activity of a SIGLEC-BMS protein. The preferred antagonist selectively antagonizes the biological activity of SIGLEC-BMS, not affecting any other cellular proteins. Further, the preferred antagonist reduces SIGLEC-BMS activity by more than 50%, more preferably by more than 90%, most preferably eliminating all SIGLEC-BMS activity.

As used herein, an agent is said to agonize SIGLEC-BMS activity when the agent increases the biological activity of a SIGLEC-BMS protein. The preferred agonist selectively agonizes the biological activity of SIGLEC-BMS, not affecting any other cellular proteins. Further, the preferred antagonist increases SIGLEC-BMS activity by more than 50%, more preferably by more than 90%, most preferably more than doubling SIGLEC-BMS activity.

Another embodiment of the assays of the invention includes screening agents and cellular constituents that bind to SIGLEC-BMS proteins using a yeast two-hybrid system (Fields, S. and Song, O., supra) or using a binding-capture assay (Harlow, supra). Generally, the yeast two-hybrid system is performed in a yeast host cell carrying a reporter gene, and is based on the modular nature of the GAL transcription factor which has a DNA binding domain and a transcriptional activation domain. The two-hybrid system relies on the physical interaction between a recombinant protein that comprises the DNA binding domain and another recombinant protein that comprises the transcriptional activation domain to reconstitute the transcriptional activity of the modular transcription factor, thereby causing expression of the reporter gene. Either of the recombinant proteins used in the two-hybrid system can be constructed to include the SIGLEC-BMS-encoding sequence to screen for binding partners of SIGLEC-BMS. The yeast two-hybrid system can be used to screen cDNA expression libraries (G. J. Hannon, et al. 1993 Genes and Dev. 7: 2378-2391), and random aptmer libraries (J. P. Manfredi, et al. 1996 Molec. And Cell. Biol. 16: 4700-4709) or semi-random (M. Yang, et al. 1995 Nucleic Acids Res. 23: 1152-1156) aptmers libraries for SIGLEC-BMS ligands.

SIGLEC-BMS proteins which are used in the screening assays described herein include, but are not limited to, an isolated SIGLEC-BMS protein, a fragment of a SIGLEC-BMS protein, a cell that has been altered to express a SIGLEC-BMS protein, or a fraction of a cell that has been altered to express a SIGLEC-BMS protein.

The candidate agents to be tested for binding with SIGLEC-BMS proteins and/or modulating the activity of SIGLEC-BMS proteins can be, as examples, peptides, antibody, small molecules, and vitamin derivatives, as well as carbohydrates. A skilled artisan can readily recognize that there is no limit as to the structural nature of the agents tested for binding to SIGLEC-BMS proteins. One class of agents is peptide agents whose amino acid sequences are chosen based on the amino acid sequence of the SIGLEC-BMS protein. Small peptide agents can serve as competitive inhibitors of SIGLEC-BMS protein.

Candidate agents that are tested for binding with SIGLEC-BMS proteins and/or modulating the activity of SIGLEC-BMS proteins are randomly selected or rationally selected. As used herein, an agent is said to be randomly selected when the agent is chosen randomly without considering the specific sequences of the SIGLEC-BMS protein. Examples of randomly selected agents are members of a chemical library, a peptide combinatorial library, constituents of a growth broth of an organism, or plant extract.

As used herein, an agent is said to be rationally selected when the agent is chosen on a nonrandom basis that is based on the sequence of the target site (SIGLEC-BMS protein) and/or its conformation in connection with the agent's action. Agents are rationally selected by utilizing the peptide sequences that make up the SIGLEC-BMS protein. For example, a rationally selected peptide agent can be a peptide whose amino acid sequence is identical to a selected fragment of a SIGLEC-BMS protein.

The cellular extracts to be tested for binding with SIGLEC-BMS proteins and/or modulating the activity of SIGLEC-BMS proteins are, as examples, aqueous extracts of cells or tissues, organic extracts of cells or tissues or partially purified cellular fractions. A skilled artisan can readily recognize that there is no limit as to the source of the cellular extracts used in the screening methods of the present invention.

Pharmaceutical Compositions of the Invention

The invention includes pharmaceutical compositions for use in the treatment of immune system diseases comprising pharmaceutically effective amounts of soluble SIGLEC-BMS molecules. The pharmaceutical composition can include soluble SIGLEC-BMS protein molecules and/or nucleic acid molecules, and/or vectors encoding the molecules. In a preferred embodiment, the soluble SIGLEC-BMS protein molecule has the amino acid sequence of the extracellular domain of SIGLEC-10 as shown in either FIG. 6B. The compositions may additionally include other therapeutic agents, including, but not limited to, drug toxins, enzymes, antibodies, or conjugates.

In one embodiment, the pharmaceutical compositions may comprise a SIGLEC antibody, either unmodified, conjugated to a therapeutic agent (e.g., drug, toxin, enzyme or second antibody) or in a recombinant form (e.g., chimeric or bispecific). The compositions may additionally include other antibodies or conjugates (e.g., an antibody cocktail).

The pharmaceutical compositions also preferably include suitable carriers and adjuvants which include any material which when combined with the SIGLEC-BMS molecules of the invention retains the molecule's activity and is non-reactive with the subject's immune system. Examples of suitable carriers and adjuvants include, but are not limited to, human serum albumin; ion exchangers; alumina; lecithin; buffer substances, such as phosphates; glycine; sorbic acid; potassium sorbate; and salts or electrolytes, such as protamine sulfate. Other examples include any of the standard pharmaceutical carriers such as a phosphate buffered saline solution; water; emulsions, such as oil/water emulsion; and various types of wetting agents. Other carriers may also include sterile solutions; tablets, including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods. Such compositions may also be formulated within various lipid compositions, such as, for example, liposomes as well as in various polymeric compositions, such as polymer microspheres.

The pharmaceutical compositions of the invention can be administered to a subject using conventional modes of administration including, but not limited to, intravenous (i.v.) administration, intraperitoneal (i.p.) administration, intramuscular (i.m.) administration, subcutaneous administration, oral administration, administration as a suppository, or as a topical contact, or the implantation of a slow-release device such as a miniosmotic pump. The pharmaceutical compositions of the invention may be in a variety of dosage forms, which include, but are not limited to, liquid solutions or suspensions, tablets, pills, powders, suppositories, polymeric microcapsules or microvesicles, liposomes, and injectable or infusible solutions. The preferred form depends upon the mode of administration and the therapeutic application.

The most effective mode of administration and dosage regimen for the compositions of this invention depends upon many factors including, but not limited to the type of tissue affected, the type of autoimmune disease being treated, the severity of the disease, a subject's health, and a subject's response to the treatment with the agents. Accordingly, dosages of the agents can vary depending on the subject and the mode of administration.

The soluble SIGLEC-BMS molecules may be administered to a subject in an appropriate amount and for a suitable time period (e.g. length of time and/or multiple times). Administration of the pharmaceutical compositions of the invention can be performed over various times. In one embodiment, the pharmaceutical compositions of the invention can be administered for one or more hours. In addition, the administration can be repeated depending on the severity of the disease as well as other factors as understood in the art.

The following examples are presented to illustrate the present invention and to assist one of ordinary skill in making and using the same. The methodology and results may vary depending on the intended goal of treatment and the procedures employed. The examples are not intended in any way to otherwise limit the scope of the invention.

### EXAMPLE 1

The following provides a description of the methods used to obtain the Siglec-BMS cDNA clones, the sequences of the cDNA clones and the SIGLEC-BMS polypeptides.

The nucleic acid molecules having Siglec-BMS nucleotide sequences were obtained by searching a proprietary ESTdatabase (Incyte EST database, Palo Alto, Calif.) for human gene sequences that exhibit elevated transcript expression in diseased immune tissues compared to normal tissues, identifying the cDNA clones of interest, acquiring the clones from the proprietor of the database (Incyte), and sequencing the entire insert of the clones. In particular, the search identified a nucleotide sequence, Siglec-BMS (-L3a) that is preferentially expressed in eosinophils from an asthmatic patient. Other cDNA clones having the nucleotide sequences of Siglec-BMS (-L3b, -L3c, -L3d, -L4a, -L5a, and -L5b) were obtained by further mining of the same ESTdatabase and acquiring the cDNA clones.

DNA from individual cDNA clones was isolated using a Qiagen BioRobot 9600. The purified DNA was then cycle sequenced using dye terminator chemistries and subsequently separated and detected by electrophoresis through acrylamide gels run on ABI 377 sequencers (Perkin-Elmer).

The nucleotide sequences of the Siglec-BMS cDNA clones were analyzed in all 3 open reading frames (ORFs) on both strands to determine the predicted amino acid sequence of the encoded protein. The nucleotide sequence analysis was performed using SeqWeb version 1.1 (GCG, Genetics Computer Group Wisconsin Package Version 10, Madison, Wis., 1999) using the Translate Tool to predict the amino acid sequences, and using the Structure Analysis Tool for predicting the motifs. Several Ig-like domains were identified in all clones which allowed for further similarity analysis using the Pileup Tool in GCG (Unix version 9.1, 1997). One additional Ig domain was identified in the L3 clones, based on this similarity analysis. A comparison of the amino acid sequences of each clone suggested that these cDNA clones included sequences that encoded proteins having sequence homology with human CD33 (Siglec-3). These nucleotide sequences were designated Siglec-BMS (SEQ ID NOS:1-7, 15) and the proteins sequences (SEQ ID NOS:8-14, 16) were designated SIGLEC-BMS.

A web-based lab management data system, PHRED, was used to track and process the sequence data (Ewing, B., Hillier, L., Wendl, M., and Green, P. 1998 Genome Research 8:175-185 “Basecalling of automated sequencer traces using PHRED. I. Accuracy assessment”), and the PHRAP algorithm was used for assembly of separate sequences into contiguous pieces (Ewing, B., and Green, P. 1998 Genome Research 8:186-194 “Basecalling of automated sequencer traces using PHRED. II. Error probabilities”). The assembled DNA data was edited using CONSED (Gordon, D., Abajian, C. and Green, P. 1998 Genome Research 8:195-202 “Consed: A graphical tool for sequence finishing”) to manually inspect quality and to design primers for closing sequence gaps and achieving contiguity, as well as to resolve any ambiguities within the sequence.

The Amino Acid Sequence of SIGLEC-BMS-L3a

The nucleotide sequence of Siglec-BMS-L3a (SEQ ID NO.:1, FIG. 2A, clone 526604), is predicted to represent a differentially spliced form of a Siglec-BMS-L3 transcript. The Siglec-BMS-L3a nucleotide sequence encodes an open reading frame of 584 amino acids in length that exhibits structural properties shared by CD33. This nucleotide sequence encodes the SIGLEC-BMS-L3a protein having the amino acid sequence described in SEQ ID NO.: 8 (FIG. 2B). SIGLEC-BMS-L3a includes an N-terminal 42 amino acids hydrophobic signal peptide, a 397 amino acid extracellular domain including three Ig-like domains, a 25 amino acid residue transmembrane domain, and a 120 amino acid intracellular domain which includes two putative ITIM motifs.

SIGLEC-BMS-L3a is expressed in eosinophils of an asthmatic patient; therefore, SIGLEC-BMS-L3a may be a cell-surface receptor that regulates adhesion and generates intracellular signals to direct eosinophil maturation, recruitment, and activation in sites of inflammation. Thus, SIGLEC-BMS-L3a may prove to be a potential target for asthma and other diseases of the immune system.

The Amino Acid Sequence of SIGLEC-BMS-L3b

The nucleotide sequence of Siglec-BMS-L3b, as described by SEQ ID NO.:2 (FIG. 3A, clone 527595), and represents a partial transcript that is related to Siglec-BMS-L3a. The Siglec-BMS-L3b nucleotide sequence encodes an ORF of 620 amino acids in length that exhibits structural properties shared by CD33 but lacks the first 17 amino acid residues compared to the sequence of SIGLEC-BMS-L3a. This nucleotide sequence encodes the SIGLEC-BMS-L3b protein having the amino acid sequence described in SEQ ID NO.: 9 (FIG. 3B) that includes an incomplete N-terminal 15 amino acid hydrophobic signal peptide, a 475 amino acid extracellular domain including three Ig-like domains, an amino acid insert sequence that is not found in SIGLEC-BMS-L3a, a 25 amino acid residue transmembrane domain, and a 120 amino acid intracellular domain which includes two putative ITIM motifs.

The Amino Acid Sequence of SIGLEC-BMS-L3c

The nucleotide sequence of Siglec-BMS-L3c, as described by SEQ ID NO.:3 (FIG. 4A, clone 652995), represents a partial transcript that is related to Siglec-BMS-L3a. The Siglec-BMS-L3c nucleotide sequence encodes an ORF of 573 amino acids in length that exhibits structural properties shared by CD33 but lacks the first 122 amino acid residues compared to the sequence of SIGLEC-BMS-L3a. This nucleotide sequence encodes the SIGLEC-BMS-L3c protein (SEQ ID NO.: 10, FIG. 4B) that includes an incomplete extracellular domain 428 amino acid residues in length including three Ig-like domains, a 58 amino acid insert sequence that is found in SIGLEC-BMS-L3d but not found in SIGLEC-BMS-L3b, a 25 amino acid residue transmembrane domain, and a 120 amino acid intracellular domain which includes two putative ITIM motifs.

The Amino Acid Sequence of SIGLEC-BMS-L3d

The nucleotide sequence of Siglec-BMS-L3d, as described by SEQ ID NO.:4 (FIG. 5A, clone 1709963), represents a partial transcript that is related to Siglec-BMS-L3a The Siglec-BMS-L3d nucleotide sequence encodes an ORF of 431 amino acids in length that exhibits structural properties shared by CD33 but lacks the first 45 amino acid residues compared to the sequence of SIGLEC-BMS-L3a, and lacks the sequences that encodes the C-terminal motifs. This nucleotide sequence encodes the SIGLEC-BMS-L3d protein (SEQ ID NO.: 11, FIG. 5B) which is 410 amino acid residues in length including, an incomplete extracellular domain, four Ig-like domains, and a 20 amino acid residue transmembrane domain.

The Amino Acid Sequence of SIGLEC-BMS-L4a

The nucleotide sequence of Siglec-BMS-L4a, as described by SEQ ID NO.:5 (FIG. 7A, clone 2895823), represents a differentially spliced form of a Siglec-8 transcript. The Siglec-BMS-L4a nucleotide sequence encodes an open reading frame of 467 amino acids in length that exhibits structural properties shared by CD33 but lacks an unknown number of N-terminal amino acid residues. This nucleotide sequence encodes the SIGLEC-BMS-L4a protein (SEQ ID NO.: 12, FIG. 7B) that includes, a 267 amino acid extracellular domain including two Ig-like domains, a 24 amino acid residue transmembrane domain, and a 30 amino acid intracellular domain which includes putative ITIM or ITAM motifs.

The Amino Acid Sequence of SIGLEC-BMS-L5a

The nucleotide sequence of Siglec-BMS-L5a, as described by SEQ ID NO.:6 (FIG. 8A, clone 3344926), represents a full-length cDNA clone of a differentially spliced form of a Siglec-9 transcript. The Siglec-BMS-L5a nucleotide sequence encodes an open reading frame of 464 amino acids in length that exhibits structural properties shared by CD33. This nucleotide sequence encodes the SIGLEC-BMS-L5a protein (SEQ ID NO.: 13, FIG. 8B) that includes an N-terminal 15 amino acid hydrophobic signal peptide, a 262 amino acid extracellular domain including two Ig-like domains, a 24 amino acid residue transmembrane domain, and a 30 amino acid intracellular domain which includes putative ITIM or ITAM motifs.

The Amino Acid Sequence of SIGLEC-BMS-L5b

The nucleotide sequence of Siglec-BMS-L5b, as described by SEQ ID NO.:7 (FIG. 9A, clone 3403156), represents a transcript that is related to Siglec-BMS-L5a, such as a differentially spliced form of Siglec-BMS-L5a. The Siglec-BMS-L5b nucleotide sequence encodes an open reading frame of 287 amino acids in length that exhibits structural properties shared by CD33. This nucleotide sequence encodes the SIGLEC-BMS-L5b protein (SEQ ID NO.: 14, FIG. 9B) that includes an N-terminal 15 amino acid hydrophobic signal peptide, a 155 amino acid extracellular domain including only one Ig-like domain, and an insert having a sequence not found in SIGLEC-BMS-L5b which shifts the reading frame of the C-terminal end of this protein. The sequence of SIGLEC-BMS-L5b lacks a transmembrane domain.

### EXAMPLE 2

The following provides a description of analysis of the expression patterns of Siglec-BMS transcripts in various human tissues using Northern blot techniques.

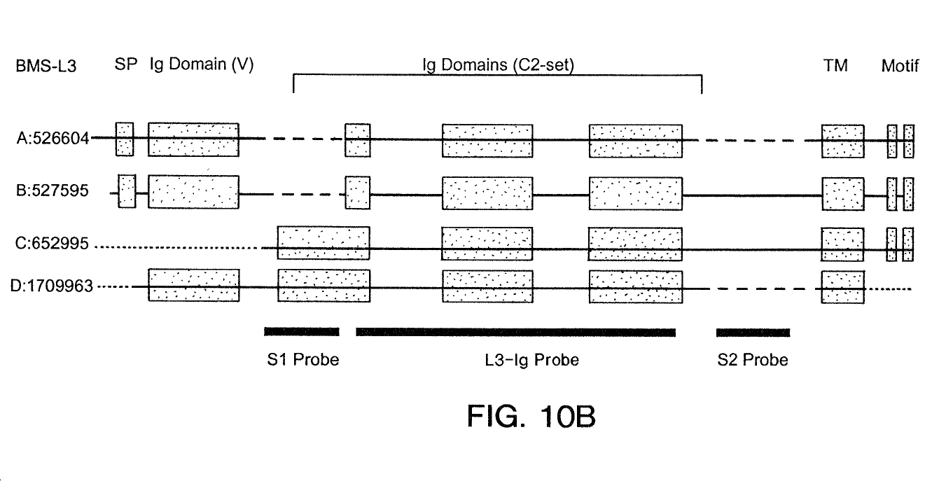

Northern blot membranes (FIG. 10A) were obtained from Clontech (MTN Blots, Clonetech, Palo Alto, Calif.). Each lane of the membrane contained approximately 1-2 micrograms of poly A+ RNA extracted from various human tissues. Blots including RNA samples from human spleen, lymph node, thymus, PBL, bone marrow, and fetal liver (MTN Human Immune System II blot) and blots including RNA samples from human brain, heart, skeletal muscle, colon, thymus, spleen, kidney, liver, small intestine, placenta, lung, and PBL (MTN Human 12 lane blot) were each hybridized with probes generated by PCR methods, using full-length Siglec-BMS-L3 as a reference sequence beginning with the start codon ATG (FIG. 10B). The L3 probe includes nucleotide sequences common in Siglec-BMS-3a, -3b, -3c, and -3d, from nucleotide position 596-1328. The SI probe includes splice variant sequences common in Siglec-BMS-3c and -3d from nucleotide position 428-593. The S2 probe includes splice variant sequences common in Siglec-BMS-3b and -3c from nucleotide position 1341-1578. All three probes were amplified from the Siglec-BMS-L3c sequence (e.g., 652995). Additionally, a β-actin probe was used as a control probe (Clontech, Palo Alto, Calif.).

PCR primers used to generate the probes for the Northern analysis included:

| L3: | |
|---|---|
| 5′ (596-616) | |
| TGC TCA GCT TCA CGC CCA GAC | (SEQ ID NO:33) |
| 3′ (1319-1328) | |
| TGC ACG GAG AGG CTG AGA GA | (SEQ ID NO:34) |
| Probe length: 732 bp | |
| S1: | |
| 5′ (428-446) | |
| CTC AGA AGC CTG ATG TCT A | (SEQ ID NO:35) |
| 3′ (576-593) | |
| GAG AAG TGG GAG GTC GTT | (SEQ ID NO:36) |
| Probe length: 65 bp | |
| S2: | |
| 5′ (1341-1359) | |
| CTG CTG GGC CCC TCC TGC | (SEQ ID NO:37) |
| 3′ (1559-1578) | |
| GAC GTT CCA GGC CTC ACA G | (SEQ ID NO:38) |
| Probe length: 237 bp | |

Reference Sequence: Full Length BMSL3 starting with ATG

The probes were individually labeled with ³²P-dCTP by random priming, purified on a Chromospin 100 column (Clontech), and heat-denatured. The membranes were pre-hybridized in ExpressHyb Solution (Clontech) at 68 degrees C. for 30 minutes with continuous shaking. The membrane was incubated with the denatured probes (approximately 2 million cpm per ml) in fresh ExpressHyb Solution for 4 hours at 68 degrees C., with continuous shaking. The membrane was washed in several changes of 2× SSC, containing 0.05% SDS, for 40 minutes at room temperature. The wash was followed by several changes of 0.1× SSC, containing 0.1% SDS, for 40 minutes at 50 degrees C. The hybridization pattern of the membranes was obtained using Phosphorlmager 445 SI (Molecular Dynamics, Sunnyvale, Calif.).

The L3 probe readily detected a 4.4 kb transcript in human immune tissues, including spleen, lymph node, and PBL. Lower levels of Siglec-BMS-L3 transcripts were also detectable in human, thymus, bone marrow, and fetal liver (FIG. 10A). The Siglec-BMS-L3 transcripts were not detected in human non-immune tissues including brain, heart, skeletal muscle, colon, kidney, liver, small intestine, placenta or lung.

### EXAMPLE 3

The following provides a description of the analysis of the expression patterns of Siglec-BMS transcripts in various human tissues using standard reverse transcriptase PCR amplification techniques.

Reverse transcriptase PCR methods were employed to determine the tissue distribution of Siglec-BMS transcripts in RNA extracted from primary human cells and cell lines and commercially available human organ cDNA. Human I, II and Immune Multiple Tissue cDNA Panels were purchased from Clontech. In addition, RNA was extracted from monocytes, TNF-stimulated endothelial cells, spleen, HL-60 cells and Jurkat cells with Trizol (Gibco BRL, Grand Island, N.Y.) according to the directions of the manufacturer.

The extracted RNA was reverse transcribed into cDNA using the following reaction mixture: 5 micro grams total RNA from each sample in 8 micro liters diethyl cyanophosphate-treated (DEPC) water, 4 micro liters 5x first strand buffer (Gibco BRL), 2 micro liters 10 mM deoxynucleotide triphosphate (dNTP) (Gibco BRL), 2 micro liters 0.1 M DTT (Gibco BRL), 1 micro liters RNAse inhibitor (40 U, Roche Molecular Biochemicals, Indianapolis, Ind.), 2 micro liters 10× hexanucleotides (Roche Molecular Biochemicals) and 1 micro liter SuperScript II (Gibco BRL). The reaction mixture was incubated at 37 degrees C. for 1 hour, then at 75 degrees C. for 15 minutes, and stored at 4 degrees C. Custom primers were obtained from Life Technologies (Gaithersburg, Md.) and sequencing parameters optimized for each primer pair. The quality of the PCR products was determined by electrophoresis on a 1.2% agarose gel. PCR was carried out using “Ready-To-Go” PCR Beads (Pharmacia Biotech Inc., Piscataway, N.J.) in a 25 micro liters reaction mixture including: 1.5 U Taq polymerase, 10 mM Tris-HCl (pH 9.0 at room temperature), 50 mM KCl, 1.5 mM MgCl₂, 200 μM of each dNTP and stabilizers, including BSA, 0.2 micro M of each primer, and 1 micro liter of RT-PCR reaction product or 2 ul of each of the commercially prepared MTC Human I, II and Immune cDNA panels from Clontech.

PCR primer sequences used for the PCR reactions included:

| P1 primers: | |
|---|---|
| 5′ (−96/−77) | |
| CCT TCG GCT TCC CCT TCT GC | (SEQ ID NO:39) |
| 3′ (560-579) | |
| CGT TGG TTT GGT TCC TTG G | (SEQ ID NO:40) |
| P2 primers: | |
| 5′ (852-870) | |
| CAC ACT GAG CTG GGT CCT G | (SEQ ID NO:41) |
| 3′ (1560-1578) | |
| GAC GTT CCA GGC CTC ACA G | (SEQ ID NO:42) |
| P3 primers: | |
| 5′ (852-870) | |
| CAC ACT GAG CTG GGT CCT G | (SEQ ID NO:43) |
| 3′ (1670-1689) | |
| GAA AAG AAG AGC CGT GAT GC | (SEQ ID NO:44) |

Expected product size for each L3 splice variant

| BMS-L3 | P1 primers | P2 primers | P3 primers |
|---|---|---|---|
| A:526604 | None | None | 547 bp |
| B:527595 | None | 727 bp | 838 bp |
| C:652995 | 675 bp | 727 bp | 838 bp |
| D:1709963 | 675 bp | None | 547 bp |

P3 primers were expected to be 552 bp, 837 bp, 837 bp and 552 bp in length, as shown in the table above.

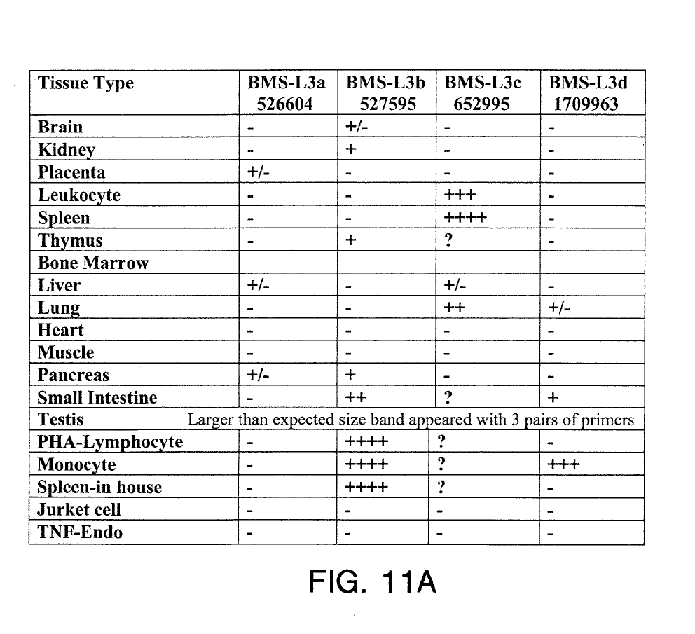

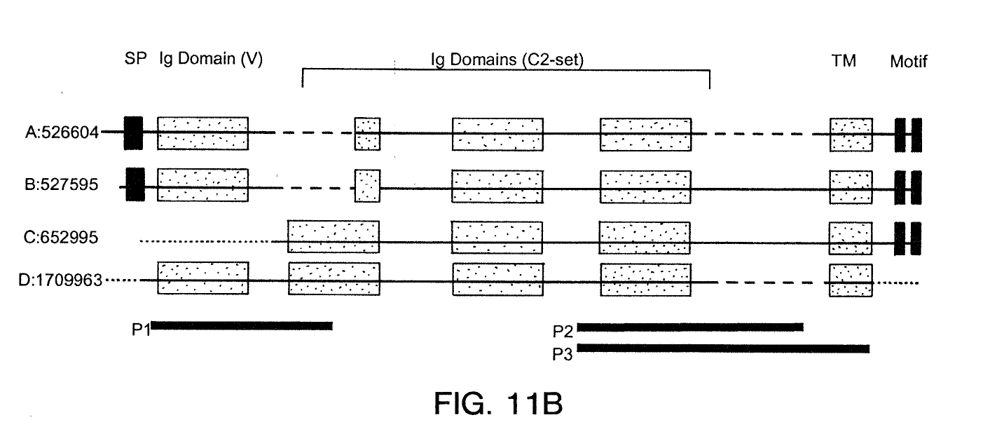

The results are shown in the table depicted in FIG. 11A.

### EXAMPLE 4

The following provides a description of the analysis of the expression patterns of Siglec-BMS transcripts in various human tissues blood cells and cell lines, using SYBR Green PCR amplification techniques.

Human I, II and Immune Multiple Tissue cDNA Panels were purchased from Clontech (Palo Alto, Calif.). In addition, RNA was obtained from lymphocytes, eosinophils, neutrophils, T-cells, monocytes, TNF-stimulated endothelial cells, spleen cells, HL60 cells and Jurkat cells and reverse transcribed as described in Example 3 above. The cDNA was amplified using the SYBR Green PCR Master Mix (PE Biosystems, Foster City, Calif.). The SYBR Green system permits relative quantification of a target transcript sequence compared to an internal house-keeping gene, beta-actin, with real-time monitoring of the amplification (PE Biosystems, User Bulletin #2 P/N 4303859). The reaction was performed on an ABI PRISM 7700 Sequence Detection System (PE Biosystems). All amplifications were normalized for beta-actin gene in the linear portion of the amplification curves.

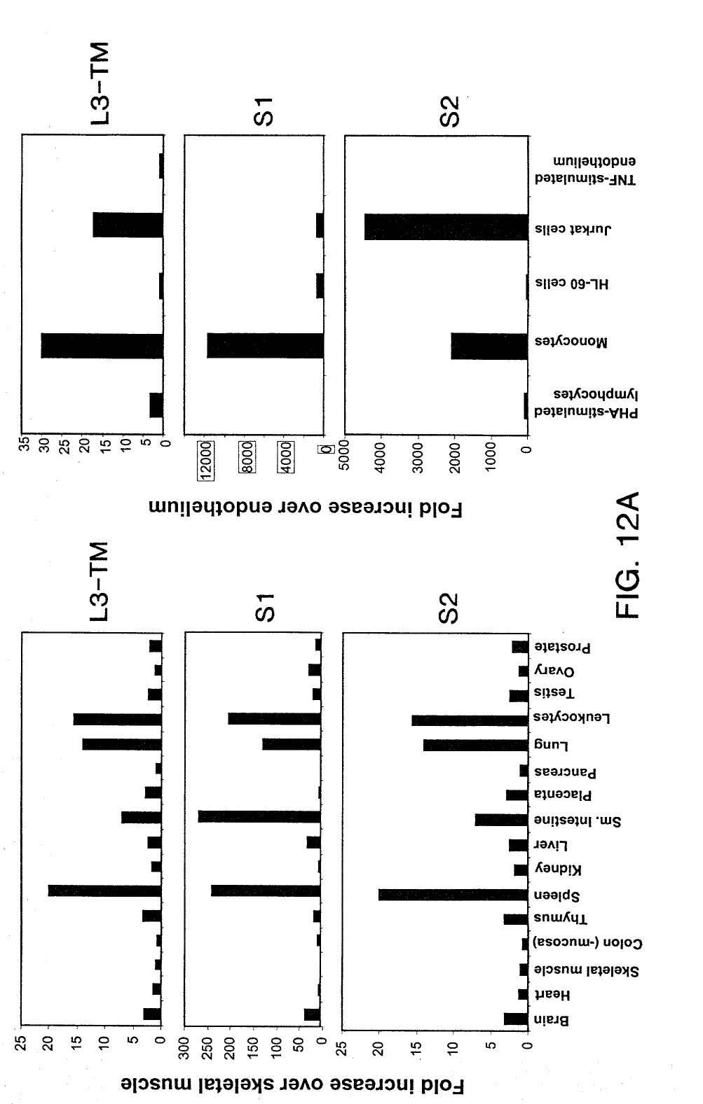

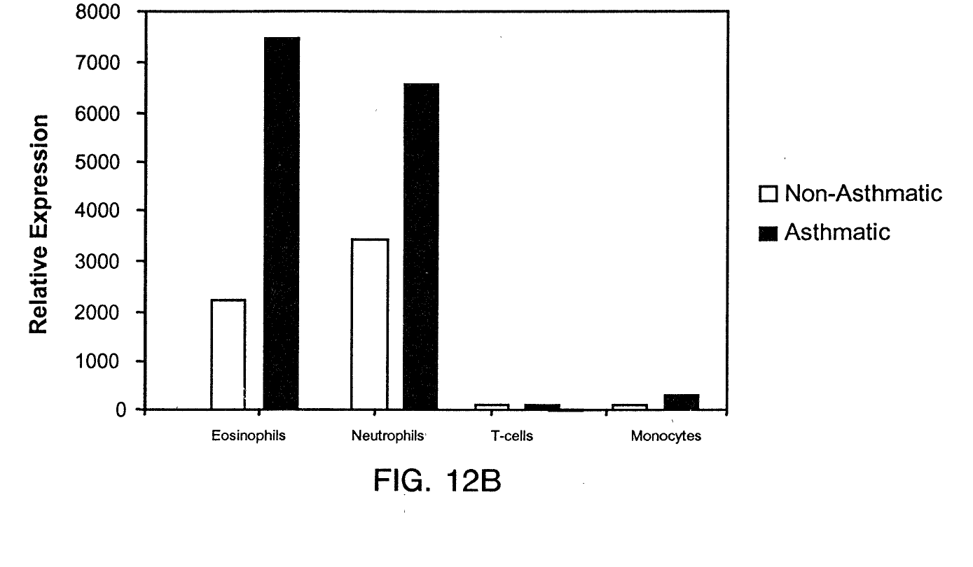

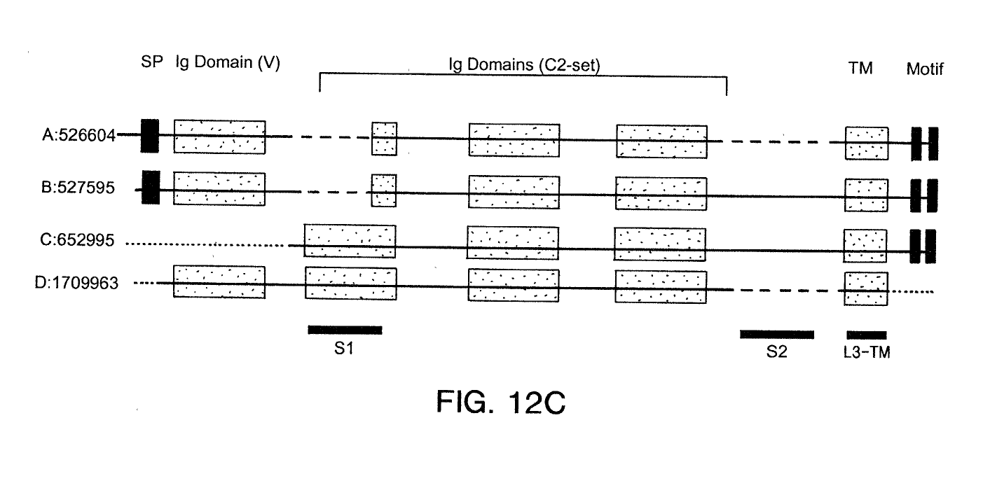

The following primer pairs were used for amplification of different regions of the L3 gene (FIG. 12A): the L3-TM primer pair includes the putative transmembrane sequences common among Siglec-BMS-3a, -3b, -3c, and -3d, from nucleotide position 1603 to 1966; the S1 primer pair includes splice variant sequences common among Siglec-BMS-3c and -3d from nucleotide position 428 to 447; and the S2 primer pair includes splice variant sequences common among Siglec-BMS-3b and -3c from nucleotide position 1948 to 1966. The data was normalized to beta-actin gene expression and then expressed as fold-increase over skeletal muscle, which served as a reference tissue (FIG. 12B).

PCR primers for the SYBR Green amplification methods included:

L3-TM:

5′ (1603-1621)

| | |
|---|---|
| TGC AGC TGC CAG ATA AGA | (SEQ ID NO:45) |

3′ (1948-1966)

| | |
|---|---|
| GGC TTG AGT GGA TGA TTT | (SEQ ID NO:46) |
| PCR product: 363 bp | |
| S1: | |

5′ (428-447)

| | |
|---|---|
| CTC CGA AGC CTG ATG TCT A | (SEQ ID NO:47) |

3′ (576-594)

| | |
|---|---|
| GAG AAG TGG GAG GTC GTT | (SEQ ID NO:48) |
| PCR product: 166 bp | |
| S2: | |

5′ (1343-1361)

| | |
|---|---|
| CTG CTG GGC CCC TCC TGC | (SEQ ID NO:49) |

3′ (1560-1579)

| | |
|---|---|
| GAC GTT CCA GGC CTC ACA G | (SEQ ID NO:50) |
| PCR product: 236 bp | |
| Beta-actin: | |
| 5′ GTG GGG CGC CCC AGG CAC CA | (SEQ ID NO:51) |
| 3′ CTC CTT AAT GTC ACG CAC GAT TC | (SEQ ID NO:52) |
| PCR product: 539 bp | |

### EXAMPLE 5

The following provides a description of the mapping of the human chromosomal location of Siglec-BMS-L3, that generated the various differentially spliced transcripts, including Siglec, BMS-L3a, -L3b, -L3c, and -L3d.

The human chromosomal map location of Siglec-BMSL3 was determined using the Stanford G3 radiation hybrid panel (Stanford University Genome Center Radiation Hybrid Mapping Server). A primer pair was chosen that would allow amplification of a 150 bp fragment from the transmembrane region. The PCR conditions included: 95 degrees C. for 5 minutes; followed by 30 cycles of 95 degrees C., 56 degrees C., 72 degrees C., for 30 seconds each; followed by 72 degrees C. for 10 minutes.

The primers were used to screen all 83 hybrids of the Stanford G3 set. The resulting pattern of positives and negatives was submitted to the Stanford Human Genome Center Radiation Hybrid Mapping Server, where it was subjected to a two-point statistical analysis against 15,632 reference markers. This analysis yielded a linkage to two markers, D19S425 and D19S418 at a distance of 32 cR [Log of Odds (LOD) score=6.47] and 29cR (LOD score=6.28), respectively, and corresponded to an approximate physical distance of 960 and 870 kb, respectively, in this panel (1 cR=30 kb). Reference to the Stanford Radiation Hybrid Map of this region of chromosome 19 gives the most likely order of D19S418-Siglec BMSL3-D19S425, with a cytogenetic location of 19q13. The marker D19S418 is positive with YAC 790A05 of the Whitehead genetic map of Chromosome 19 (Wende et al. Mammal Gen., 10, 154-160 (1999)).

PCR primers used for the chromosomal location methods included:

| | |
|---|---|
| L3-TM: | |
| 5′ (1603-1621) TGC AGC TGC CAG ATA AGA | (SEQ ID |
| | NO:53) |
| 3′ (1948-1966) GGC TTG AGT GGA TGA TTT | (SEQ ID |
| | NO:54) |
| PCR product: 363 bp | |

### EXAMPLE 6

The following provides a description of the generation of Ig fusion proteins comprising the extracellular domains of Siglec-BMS-L3a and Siglec-BMS-L3-995-2 fused to the human R gamma chain.

Plasmids encoding the Ig fusion proteins were constructed. Briefly, nucleotide sequences encoding the extracellular domains of either SIGLEC-BMS-L3a (e.g., 526604) or SIGLEC-BMS-L3-995-2 were amplified from a liver cDNA library (Clontech) by PCR methods. The nucleotide sequence encoding the extracellular domain of SIGLEC-BMS was operatively ligated into a proprietary expression vector, pd19 (Bristol-Myers Squibb, Princeton, N.J.). The pd19 vector has a cytomegalovirus promoter (CMV promoter; Boshart, M. et al., 1985 Cell 41:521-530) to drive expression of Siglec-BMS-L3a and Siglec-BMS-L3-995-2 sequences. Additionally, the pd19 vector includes a portion of the human R gamma chain having a point mutation which reduces Fc receptor binding of the immunoglobulin portion encoded therein. The resulting plasmids were designated SiglecL3A-hIg and SiglecL3-hIg. The SiglecL3-hIg fusion proteins were expressed in COS cells by DEAE-transient transfection. The fusion protein was purified from COS7 supernatant by chromatography using Protein A trisacryl column (Pierce, Rockford, Ill.). before use.

### EXAMPLE 7

The following provides a description of the determination of the binding specificity of the extracellular domain of SIGLEC-BMS-L3A and SIGLEC-BMS-L3 fusion proteins, using FACs analysis.

Mixed white blood cell populations and hemopoietic cell lines were obtained to determine the binding specificities of SIGLEC-BMS-L3A and SIGLEC-BMS-L3. The cells and cell lines used in this analysis included the following: cell lines MB, PM, and TJ which are EBV transformed B-cells (Bristol-Myers Squibb); B-cell lymphoblastomas Ramos, HSB-2, and Raji; Jurkat which is a T-cell lymphoblastoma; HEL which is a erythroblastic leukemia cell line HEL; and monocytic cell lines U973 and HL60 which were obtained from the American Type Culture Collection (Manassas, Va.).

The cells were suspended in binding buffer (DMEM including 1% w/v bovine serum albumin and 0.1% sodium azide), with the Siglec fusion protein (Example 6), mALCAM hIg fusion protein (R-gamma fusion protein control), or CD5 hIg fusion protein (E-gamma fusion protein control) at a concentration of 5 micro grams of protein/1×10⁶ cells. Rabbit Ig (Sigma Chemical Co., St. Louis, Mo.) was also added at 100 micro grams/million cells to prevent non-specific binding of the Ig tail on the fusion proteins to the Fc receptors. The mixture was incubated on ice for 1 hour followed by two washings with binding buffer. The cells were centrifuged at 500× G for 5 minutes between each wash. Anti-hIg/FITC (Jackson Immunoresearch, West Grove, Pa.) and/or phycoerythrin-conjugated anti-CD20/PE (Beckton Dickenson, San Jose, Calif.), anti-CD14/PE (Beckton-Dickenson), and anti-CD4/PE (Beckton-Dickenson) were added on ice for 30 minutes. After further washing, the cells were analyzed on a Becton Dickenson FACSort using Cell Quest software. Cells were live gated and red/green color was compensated.

The mixed white blood cell populations were analyzed for binding to the SIGLEC-BMS fusion proteins. Results of FACs analysis are shown in Table 1. There was no difference in the binding of SIGLEC BMSL3a and SIGLEC BMSL3 to the cells that were examined by FACs. A small population of lymphocyte-sized cells and monocyte-sized cells stained positively for both fusion proteins. Double staining with either anti-CD20 (for B-cells), anti-CD-14 for monocytes, anti-CD4 or anti-CD3 (for T-cells) determined that B-cells and monocytes were binding the fusion protein, but T-cells were not. Possible binding of the fusion protein to the Fc receptors on B-cells and monocytes was ruled out by comparison with two fusion protein controls, one with a similar R-gamma hIg tail that doesn't bind FcR (mALCAM hIg) and one with an E-gamma hIg tail that does bind FcR (CD5 hIg).

Similar FACs analyses were performed with cell lines. The HEL (e.g., an erythroblastic leukemia cell line) and Jurkat (e.g., a T-cell line) cell lines did not stain positively for either SIGLEC BMSL3 fusion protein. Additionally, the EBV-transformed B cell lines MB, PM and TJ did not stain positively. The B-cell lines, Ramos, Raji and HSB2, did stain positively. Although some monocyte binding was observed in whole blood, the monocytic cell lines, U973 and HL60, did not exhibit any binding.

Table 1 is a FACs analysis of Siglec-10-hIg binding. Data was obtained by incubating mixed white blood cell populations and hemapoietic cell lines with Siglec-10-hIg fusion protein then stained with fluorescein-conjugated anti-hIg (Jackson Immunoresearch, West Grove, Pa.) and/or phycoerythrin-conjugated anti-CD20, anti-CD3, anti-CD14, and anti-CD4. mALCAM hIg fusion protein (hIg Rγ control) and CD5 hIg fusion protein (hIg E7 control) were analyzed in parallel as controls. Rabbit Ig (Sigma) was also added to prevent non-specific binding of the Ig tail on the fusion proteins to Fc receptors. The percentage of cells staining positive for FITC compared to background with mALCAM, CD5 and Siglec-10 hIg is shown. One color FACs was used for cell lines and two color FACs was used for primary peripheral blood mononuclear cells (PBMC).

TABLE 1

| | | FITC Staining (%) | | |
|---|---|---|---|---|
| | | mALCAM | CD5 | |
| Cell line | Type | hIg | hIg | Siglec-10-hIg |
| MB | B-cell (EBV) | 0 | 0 | 0 |
| PM | B-cell (EBV) | 0 | 0 | 0 |
| TJ | B-cell (EBV) | 0 | 0 | 0 |
| Ramos | B-cell (lymphoma) | 0 | 4 | 57 |
| HSB-2 | B-cell (lymphoma) | 0 | 0 | 24 |
| Raji | B-cell (lymphoma) | 1 | 2 | 36 |
| Daudi | B-cell (lymphoma) | 0 | 0 | 20 |
| Jurkat | T-cell (lymphoma) | 38 | 2 | 0 |
| HEL | RBC (leukemia) | 0 | 0 | 0 |
| U973 | Monocyte (leukemia) | 0 | 4 | 0 |
| HL60 | Monocyte (leukemia) | 0 | 0 | 0 |
| | | FITC Staining (%) | | |
| Blood | | ALCAM | CD5 | |
| Population | PE+ (%) | hIg | hIg | Siglec-10-hIg |
| PBMC | | | | |
| CD20+ | 7 | 0 | 0 | 4 |
| CD14+ | 11 | 0 | 0 | 8 |
| CD4lo+ | 12 | 0 | 0 | 11 |
| CD4hi+ | 63 | 8 | 0 | 0 |
| CD3+ | 65 | 4 | 0 | 0 |
| Granulocytes | | 0 | 0 | 0 |

### EXAMPLE 8

The following provides a description of the determination of whether distinct blood cell populations or cell lines exhibit binding specificity for the extracellular domain of SIGLEC-BMS-L3 fusion protein, using a solid support method.

The SIGLEC-BMS-L3 fusion protein was immobilized on a solid support, by coating an ELISA plate with SIGLEC-BMSL3 hIg fusion protein (200 ng/well) overnight. The plate was blocked for 1 hour with DMEM containing 1% BSA.

The cells and cell lines used included: mixed white blood cells, mixed granulocytes, purified B-cells, purified NK cells, purified monocytes, and Ramos (B-cell line), RBCs, Jurkats (T-cell line), and HL60s and K652 (monocytic cell lines). The blood cells and cell lines were labeled with calcein-AM (5 micro liter/10⁸ cells) for 30 minutes at 37 degrees C. The cells were washed two times in Hanks buffered salt solution (HBSS) and added to the blocked ELISA plates (4×10⁵/well in 200 micro liters) at 37 degrees C. for 30 minutes. The plates were gently washed with HBSS and 100 micro liters HBSS was added to each well. Fluorescence was read on a CytoFluor 4000 (PerSeptive Biosystems, Framingham, Mass.) at 485 excitation/530 emission.

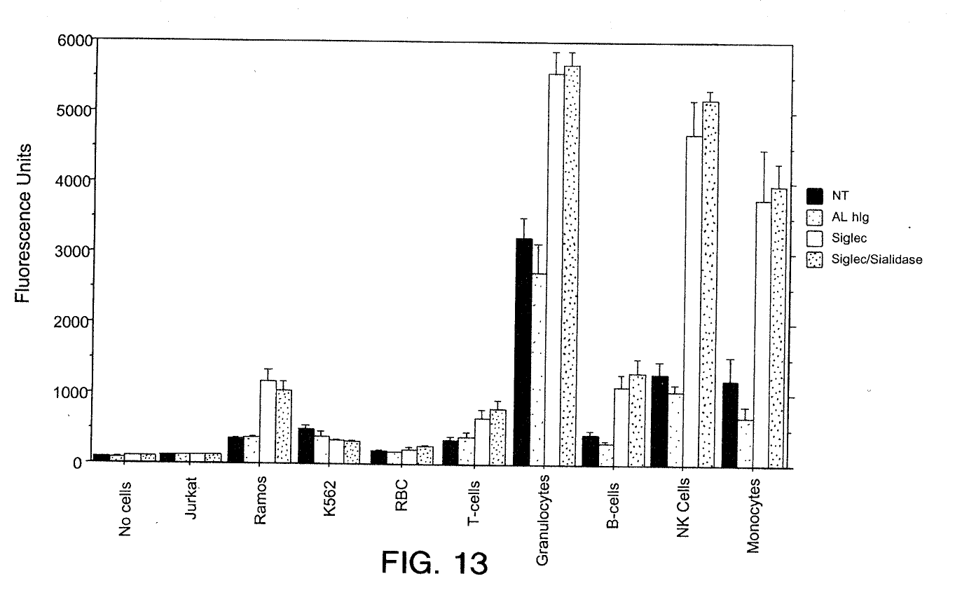

The results are shown in FIG. 13. The mixed white blood cells, mixed granulocytes, purified B-cells, purified NK cells, purified monocytes, and Ramos (B-cell line) adhered to the immobilized SIGLEC fusion protein. RBCs, Jurkats (T-cell line), HL60s and K652 (monocytic cell lines) did not adhere to the protein-coated plate. Sialidase pretreatment of the cells (0.1 U/ml for 30 minutes at 37 degrees C.) did not significantly affect binding of any of the adherent cell types.

### EXAMPLE 9

The following provides a description of the binding studies using COS cells expressing the full length SIGLEC-BMS-L3 (e.g., 995-2, see Example 14) protein and various cells and cell lines.

COS7 cells were transiently transfected or mock-transfected with a pcDNA3 plasmid (InVitrogen, Carlsbad, Calif.) containing a full length Siglec-BMSL3 (e.g., 995-2, see Example 14) by the DEAE-dextran method. Twenty four hours after transfection, the cells were lifted from the plates with EDTA and re-plated in 6-well plates containing DMEM with 10% FCS at a density of 2×10⁵/well. Binding assays were performed between 48 and 60 hours post-transfection.

Blood cells and cell lines were labeled with calcein-AM (5 micro liters/10⁸ cells) for 30 minutes at 37 degrees C. RBCs, mixed white blood cells, Ramos (B-cell line), HL60 and K562 (monocytic cell lines), and Jurkats (T-cell line) were suspended in DMEM containing 0.25% BSA. Some cells were also pre-treated with sialidase (0.1 U/ml for 30 minutes at 37 degrees C. followed by 3 washes with DMEM +0.25% BSA). One ml of blood cells or a cell line suspension was added to each well. The cells were incubated together at 37 degrees C. for 30 minutes with gentle rocking. The plates were washed gently 3 times with PBS +0.25% BSA. The cells were fixed with 0.25% glutaraldehyde.

To quantify binding, the percentage of transfected COS7 cells that bound two or more of the added cell types was determined from 10 fields in each treatment (at least 100 cells from each treatment were scored). The results were expressed as a percentage of COS7 cell binding. Binding to the transfected cells was also compared to the mock-transfected controls.

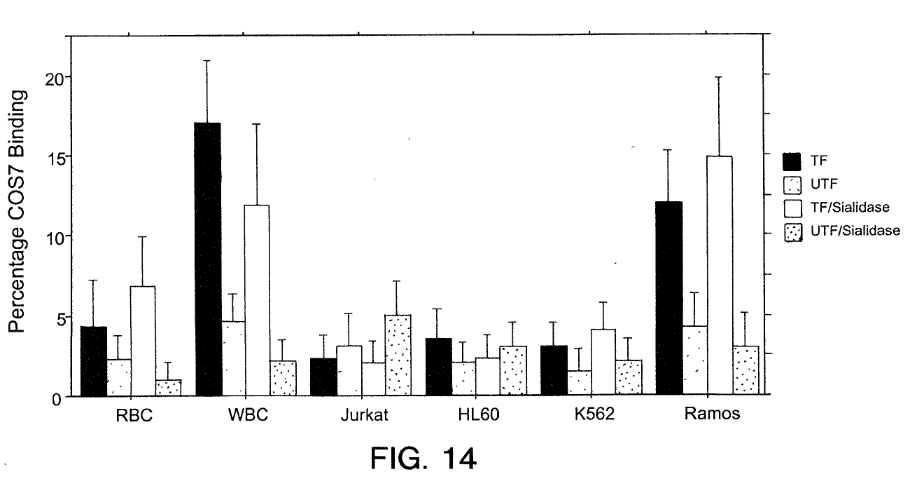

The results are shown in FIG. 14. The transfected COS7 cells bound to the mixed white blood cells and Ramos cell line (B-cell line). This binding was not significantly affected by sialidase pretreatment. Since there was no indication that the sialic acid digestion was complete, this observation is only suggestive. The transfected COS7 cells did not exhibit binding to the RBCs, Jurkats (T-cell line), or to HL60 and K562 (monocytic cell lines).

### EXAMPLE 10

The following provides a description of the generation of various fusion proteins comprising the cytoplasmic tail domain of Siglec-BMS-L3a fused to the GST protein.

To construct a nucleotide sequence encoding the fusion protein comprising the cytoplasmic tail domain of the SIGLEC-BMS-L3 protein, the cytoplasmic domain of SIGLEC-BMS-L3 was amplified from a PHA-activated Jurkat cDNA library (KRRTQTE . . . VKFQ*; e.g., see FIG. 6B). The cytoplasmic tail fragment was subcloned, via EcoRI/XhoI sites, into pGEX4T-3 (Pharmacia Biotech) which includes the GST sequenceThe resulting construct was designated GST-SiglecL3cyto (FIG. 15).

In addition, Y→F mutants were generated at positions 597, 641, and 691. GST-SiglecBMSL3cyto and the SIGLEC-BMS mutant proteins were expressed in E. coli bacteria and purified according to a Pharmacia protocol (based on the methods of Smith and Johnson, 1988 Gene 67:31-40).

PCR primers used to generate the sequence encoding the cytoplasmic tail domain of SIGLEC-BMS-L3cyto(wildtype) and the mutant SIGLECs included the following:

| | |
|---|---|
| GST-SiglecBMSL3cyto (wt) primers: | |
| 5′ GCG GCC AGG AAT TCC AAG AGA CGG ACT CAG ACA GAA | (SEQ ID NO:55) |
| 3′ GCG GCC CTC GAG TCA TTG GAA CTT GACTTC TGC | (SEQ ID NO:56) |
| GST-Sig1ecBMSL3Y641F: | |
| wt forward and reverse primers and Y641F mutagenic primers: | |
| 5′ CCA GAA TCA AAG AAG AAC CAG AAA AAG GAG TTT GAG TTG CCC AGT TTC CCA GAA CCC | (SEQ ID NO:57) |
| 3′ GGG TTC TGG GAA ACT GGG CAA CTG AA CTG CTT TTT CTG GTT CTT CTT TGA TTC TGG | (SEQ ID NO:58) |
| GST-SiglecBMSL3Y667F: | |
| wt forward and reverse primers and Y667F mutagenic primers: | |
| 5′ GAG AGC CAA GAG GAG CTC CAT TTT GCC ACG CTC AAC TTC CCA GGC | (SEQ ID NO:59) |
| 3′ GCC TGG GAA GTT GAG CGT GGC AAA ATG GAG CTC CTC TTG GCT CTC | (SEQ ID NO:60) |
| GST-SiglecBMSL3Y691F: | |
| wt forward and Y691F mutagenic reverse primers: | |
| 5′ GCG GCC CTC GAG TCA TTG GAA CTT GAC TTC TGC AAA ATC CGC CTG GGT GCC | (SEQ ID NO:61) |
| 3′ GCG GCC CTC GAG TCA TTG GAA CTT GAC TTC TGC AAA ATC CGC CTG GGT GCC | (SEQ ID NO:62) |
| GST-SiglecBMSL3Y641 alone (deletion mutant) primers: | |
| 5′ GCG GCC AGG AAT TCC ATC AAT GTG GTC CCG ACG GCT GGC | (SEQ ID NO:63) |
| 3′ GCG GCC CTC GAG TCA ATG GAG CTC CTC TTG GCT CTC | (SEQ ID NO:64) |

### EXAMPLE 11

The following provides a description of the generation of the various fusion proteins comprising the extracellular domain of Siglec-BMS-L3a or Siglec-BMS-L3 fused to human Ig sequences. The fusion proteins include Siglec-BMS-L3a hIg and Siglec-BMS-L3 hIg.

The nucleotide sequences encoding the extracellular domain of SIGLECBMS-L3a (e.g. 526604) and 995-2 were amplified using primers containing linker sequences with restriction sites for Hind III, Bgl II and NcoI. The amplified fragments (e.g., 1201 bp fragment for siglecBMS-L3a or 1650 bp fragment for Siglec BMS-L3) were digested with restriction enzymes Hind III and Bgl II, and the digested fragments were cloned into a pd19 vector (see Example 6; Bristol-Myers Squibb, Princeton, NJ) which was digested with Hind III and BamHI. The pd19 vector includes a portion of the human R gamma chain having a point mutation which reduces Fc receptor binding of the immunoglobulin portion of the encoded fusion protein. The integrity of the insertions was validated by digesting the Siglec/hIg plasmid constructs with either Hind III/Nco I to check the extracellular domain of Siglec or with Hind III/Xba I to check the entire fusion construct. The Siglec-10-hIg fusion protein was expressed in COS7 cells by DEAE-dextran transient transfection. COS7 cells were transfected with I micro gram/milli liter DNA in CMEM containing 1% DEAE-dextran (Sigma), 0.125% chloroquine (Sigma) and 10% NuSerum (Beckton Dickenson, Franklin Lakes, N.J.) for 4 hours followed by two minute treatment with 10% DMSO in phosphate buffered saline (PBS). After 4-7 days, the COS7 supernatant was removed and Siglec-10-hIg fusion protein was purified by chromatography over a protein A trisacryl column (Pierce, Rockford, Ill.).

Sequence of the primers used to construct Siglec-BMSL-3a hIg included the following:

| | |
|---|---|
| Hind III | |
| 5′ CCG CCT AAG CTT TCC CCT TCT GCC AAG AGC CCT GAG CCC TGA GCC | (SEQ ID NO:65) |
| ACT CAC AGC ACG ACC AGA GAA CAG GCC TGT CTC AGG CAG GCC CTG | |
| CGC CTC CTA TGC GGA GAT G | |
| Bgl II Nco I | |
| 3′ GAA GAT CTG AAC CAT GGT TAT AGT GCA CGG AGA GG | (SEQ ID NO:66) |

| | |
|---|---|
| Sequence of the primers used to construct Siglec-BMS-L3 hlg included the | |
| following: | |
| Hind III | |
| 5′ CCG CCT AAG CTT TCC CCT TCT GCC AAG AGC CCT GAG CCC TGA GCC | (SEQ ID NO:67) |
| ACT CAC AGC ACG ACC AGA GAA CAG GCC TGT CTC AGG CAG GCC CTG | |
| CGC CTC CTA TGC GGA GAT G | |
| Bgl II Nco I | |
| 3′ GAA GAT CTG AAC CAT GGT TAG GAG AAT GCC GTT GA | (SEQ ID NO:68) |

### EXAMPLE 12

The following provides a description of kinase assays used to determine if the cytoplasmic tail domain of SIGLEC-BMS-L3 undergoes phosphorylation by known tyrosine kinases.

The kinase assays were run in an ELISA format using representatives of the four major tyrosine kinases known to associate with receptors similar in nature to SIGLEC-BMS-L3. The tyrosine kinases tested included: lck, ZAP70, emt, and JAK3.

The GST fusion proteins and GST were coated on Immulon 2 96-well plates at 4 micro grams/ml in sodium carbonate pH 9 for 16 hours at room temperature. The GST fusion proteins included: GST-SiglecBMSL3cyto (wildtype), GST-LAT (an adapter protein with 10 tyrosines available for phosphorylation), GST-cyto-Y597F (Y→F mutation at the 597 position), GST-L3cyto-Y641F (Y→F mutation at the 641 position), GST-L3cyto-Y667F (Y→F mutation at the 667 position), L3cyto-Y691F (Y→F mutation at the 691 position), GST-L3cyto-Y641alone, and GST alone. LAT is a 36-38 kDa palmitoylated, integral membrane adapter protein expressed in T-cells, mast cells, NK cells, and megakaryocytes. Signal transduction through the T-cell receptor (TCR/CD3) involves the activation of tyrosine kinases and the subsequent phosphorylation of numerous cytoplasmic protein substrates. LAT has 10 tyrosine residues and is one of the major substrates of these many families of tyrosine kinases. Phosphorylated LAT is a good positive control because it binds many critical signaling molecules (W. Zhang, et al., 1998 Cell 92: 83; W. Zhang, et al., 1999 Immunity 10: 323).

The plates were washed and then blocked with blocking reagent (Hitachi Genetics Systems, Alameda, Calif. -). The kinase reactions were conducted in 50 microliter volumes, in kinase buffer (25 mM Hepes pH 7.0, 6.25 mM MnCl₂, 6.25 mM MgCl₂, 0.5 mM sodium vanadate, 7.5 micro M ATP) and two fold dilutions of the tyrosine kinases starting at a concentration of 0.25 micro grams/ml. The kinase reactions were incubated for 1 hour at room temperature. The plates were washed and the phospho-tyrosine content was detected with anti p-Tyr (PY99) HRP (Santa Cruz, Santa Cruz, CA) at 1:1000 and peroxidase substrate (KPL, Gaithersburg, Md.). Absorbance was detected at 650/450 nm.

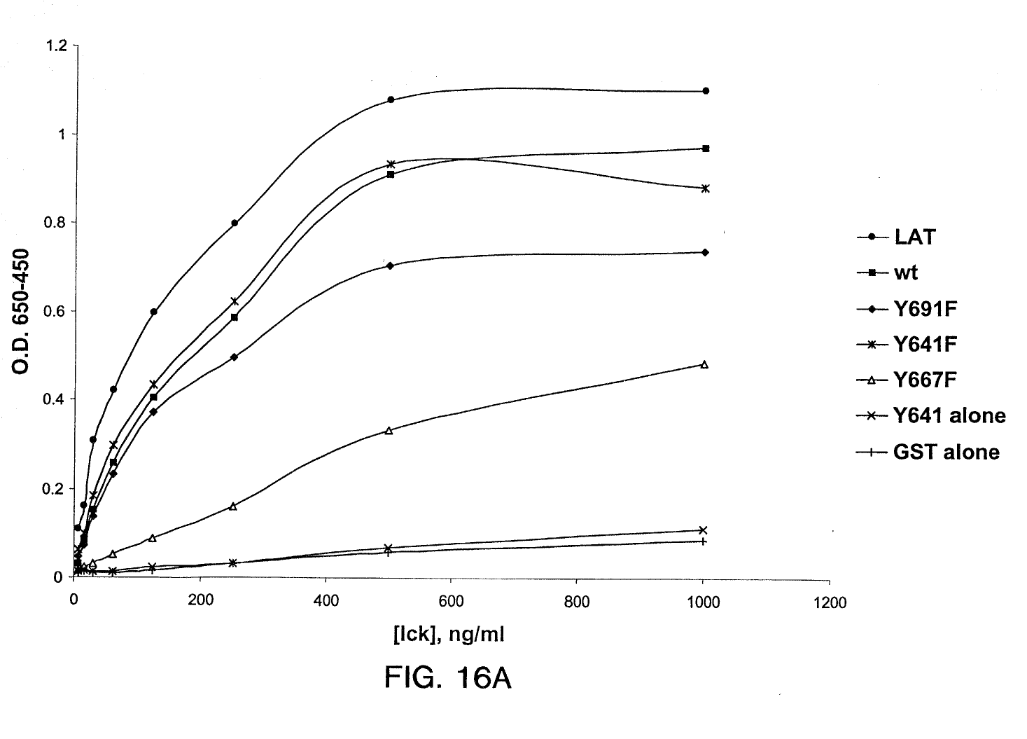

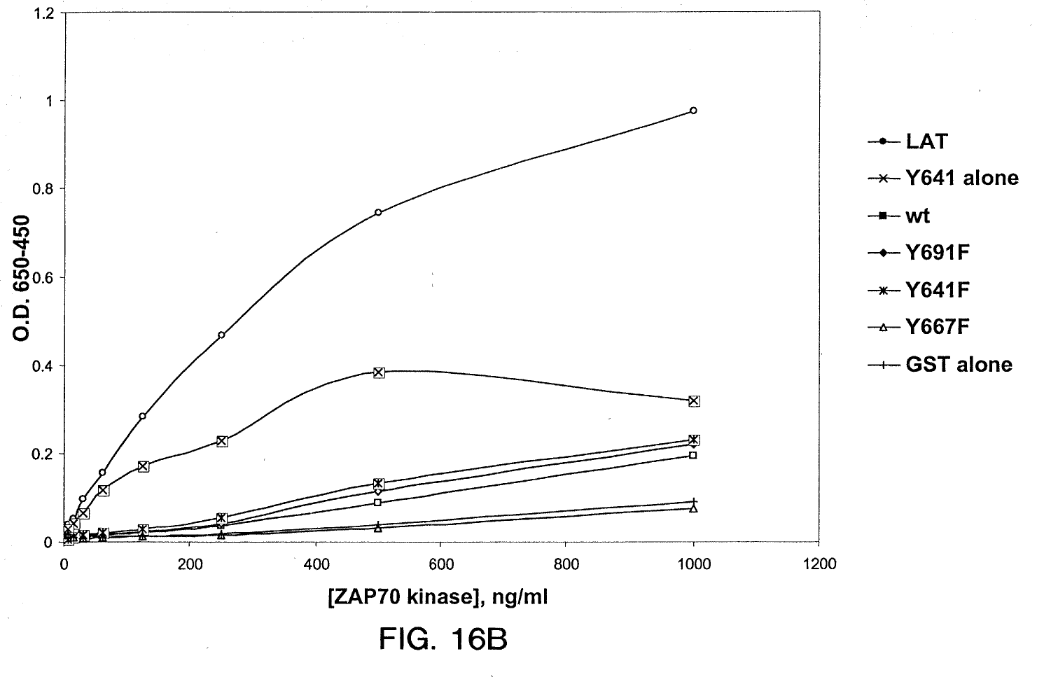

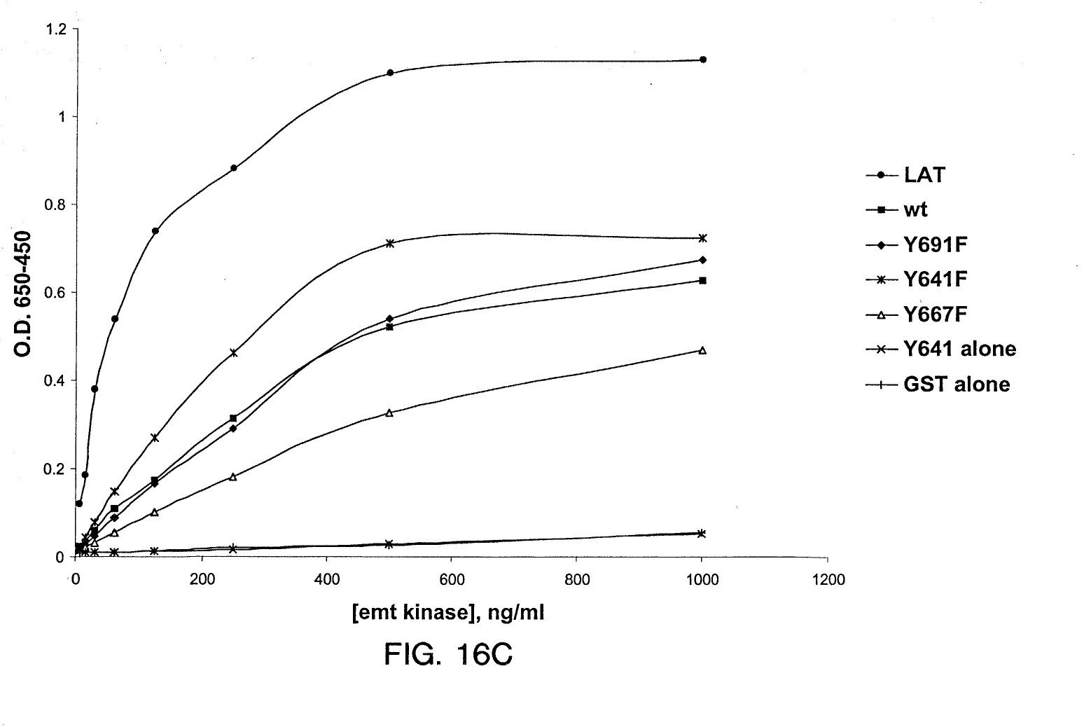

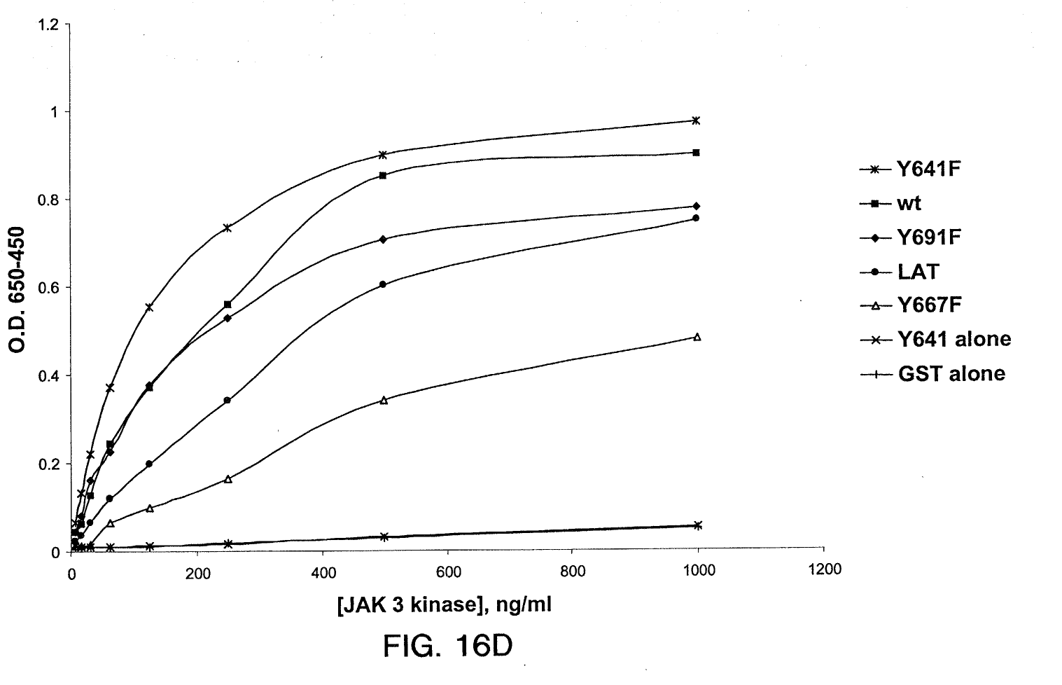

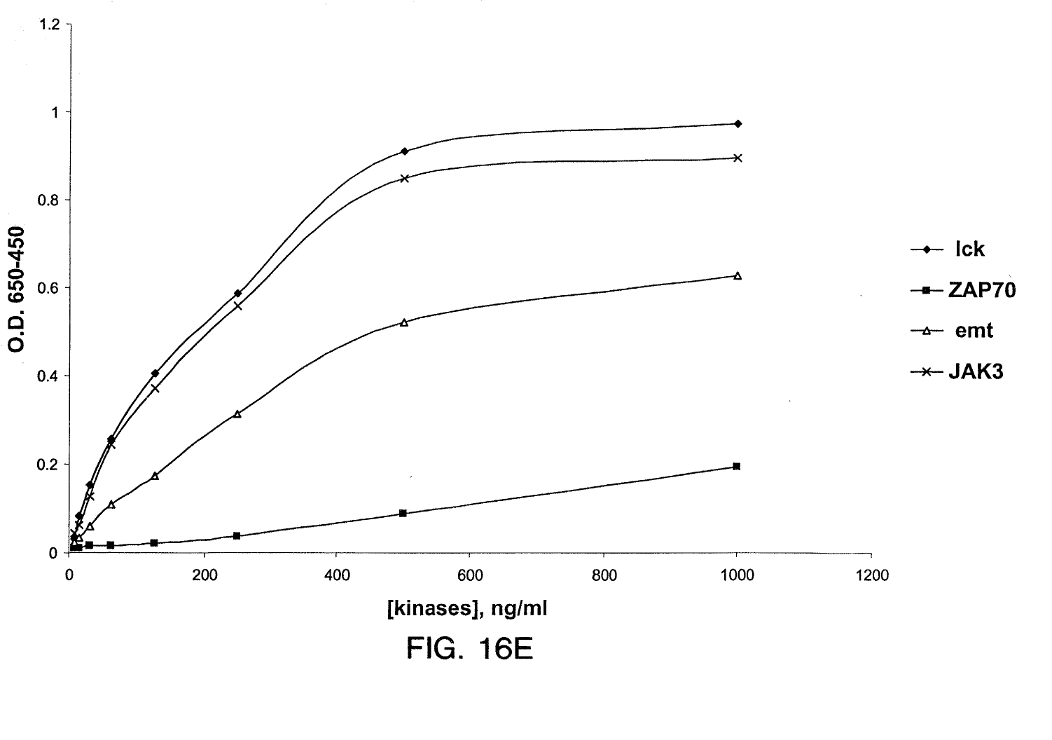

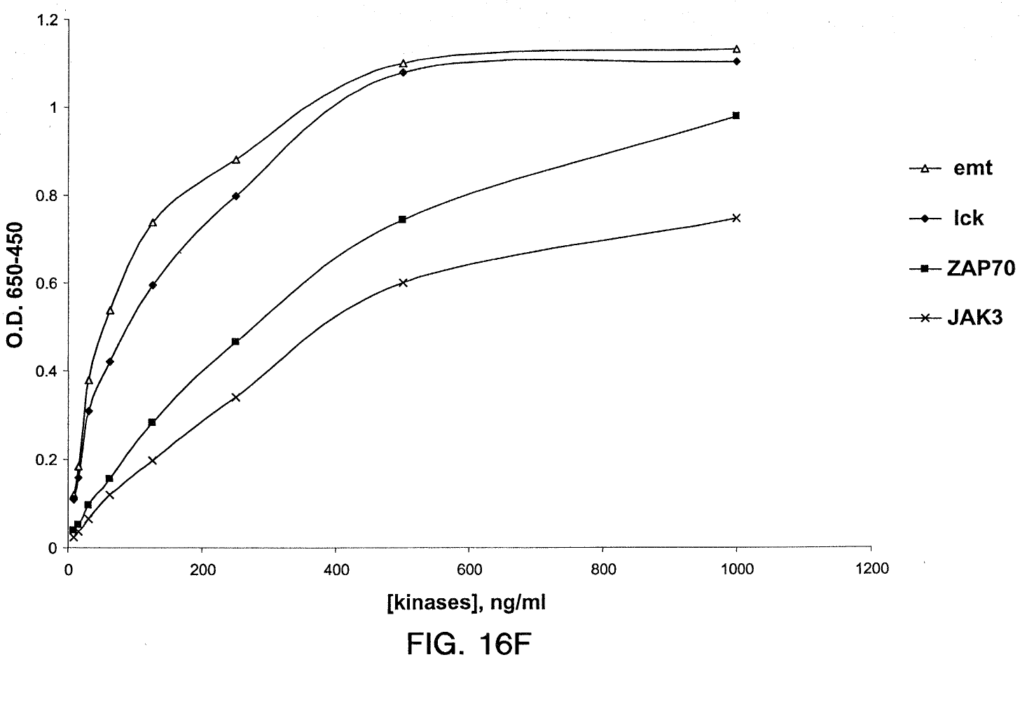

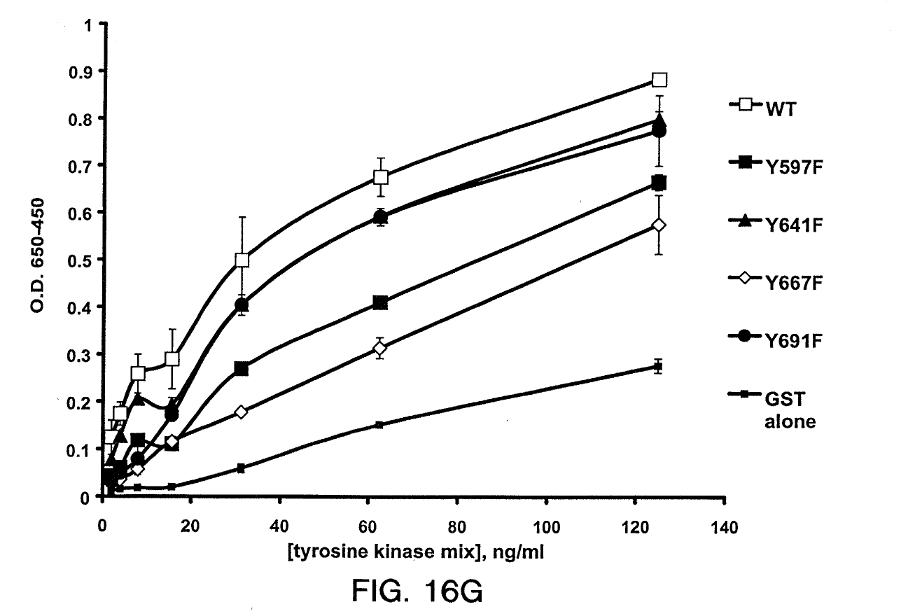

The results—of the Kinase assays shown in FIGS. 16A through G indicate that the cytoplasmic domain of the SIGLEC-BMS-L3 protein can be phosphorylated by representatives of at least three of four major families of kinases: Jak3, Lck, Emt but not ZAP-70. By titering the kinase concentration, it was determined that Siglec-10 could be phosphorylated equally well by Lck and Jak, moderate phosphorylation was observed with Emt and little or no phosphorylation occurred with ZAP-70. The wildtype GST-SIGLECBMS-L3cyto was phosphorylated by Lck(100%)>JAK3 (92%)>>emt(65%)>>>ZAP70 (20%).

The GST fusion proteins having mutations at particular positions in the cytoplasmic tail affected phosphorylation by the various tyrosine kinases. The results are summarized in the following table.

Decrease in Wild type Phosphorylation

| Mutant | lck | JAK3 | emt | ZAP70 |
|---|---|---|---|---|
| Y641F | — | — | — | — |
| Y667F | 50% | 50% | 70% | 100% |
| Y691F | 30% | 25% | — | — |
| Y641 alone | — | — | — | 20% |

The results shown in FIGS. 16A through G and in the table above suggest that the tyrosines at positions 597 and 667, contained within an ITIM-like motif, are likely targets of phosphorylation by several classes of tyrosine kinase signaling molecules, including lck, JAK3, emt and ZAP70. The tyrosine located at position 691 was also contributing to the phosphorylation of wild type Siglec tail by Lck and Jak3 kinases. For example, phosphorylation of the Y's at positions 667 and 691 accounted for approximately 80% of the wildtype phosphorylation by lck and 75% of the wildtype phosphorylation by JAK3. By comparison, the mutation of the Y at position 641 did not significantly affect the degree of phosphorylation by any of the kinases that were tested. In addition, a construct containing Y641 alone was not phosphorylated by any of the kinases, confirming that Y641 is most likely not a site for phosphorylation (data not shown). The contribution of the Y at position 597 to phosphorylation, could be calculated to be approximately 20% for lck, 25% for JAK3 and 30% for emt.

### EXAMPLE 13

The following provides a description of the use of Western blotting and ELISA techniques to determine if the cytoplasmic tail domain of SIGLEC-BMS-L3 binds SHP-1 or SHP-2 in cell lysates.

Western Blotting For SHP proteins:

To determine if the cytoplasmic domain of Siglec-10 binds SHP-1 and SHP-2 in cell lysates, 10 μg of GST fusion protein +/−tyrosine phosphorylation were incubated with 300 μl of cell lysate (Triton-X-100-soluble fraction 5×10⁷ unstimulated cells) at 4° C. overnight. The GST fusion protein complexes were captured with 50 μl of glutathione-sepharose beads (Amersham Pharmacia Biotech) for 1 hr at 4° C. The beads were then incubated with Jurkat cell lysates. The beads were washed three times with ice cold lysis buffer, and bound proteins were eluted in SDS reducing sample buffer and resolved by electrophoresis on an SDS-polyacrylamide gel. The separated proteins were transferred to nitrocellulose by standard western blotting techniques. The blots were then stained for proteins containing phosphorylated tyrosines using anti-P-Y HRP-conjugated antibody (Clone 4G10, Upstate Biotechnology, Lake Placid, N.Y.). Blots were then stripped and stained with either mouse anti-SHP-1 (Transduction Laboratories, Lexington, Ky.) or mouse anti-SHP-2 (Transduction Laboratories) followed by an HRP-conjugated secondary antibody (goat anti-mouse, Biosource Int., Camarillo, Calif.) Stained proteins were imaged by adding a chemiluminescent detection reagent (Renaissance, NEN Bio Products, Boston, Mass.) and exposing to film (Kodak).

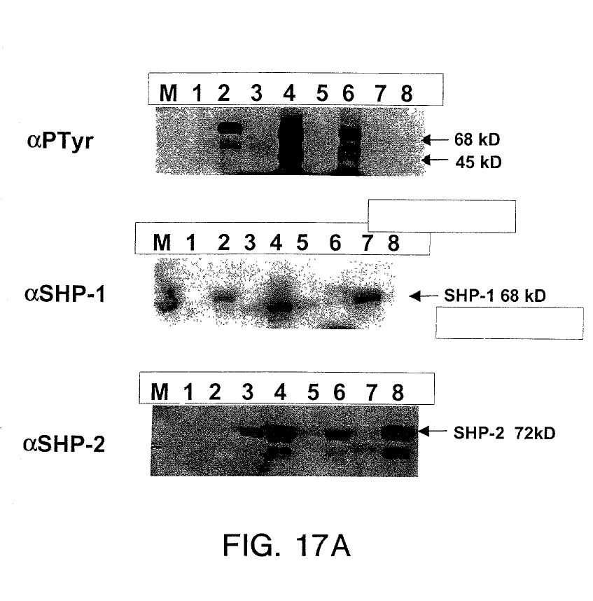

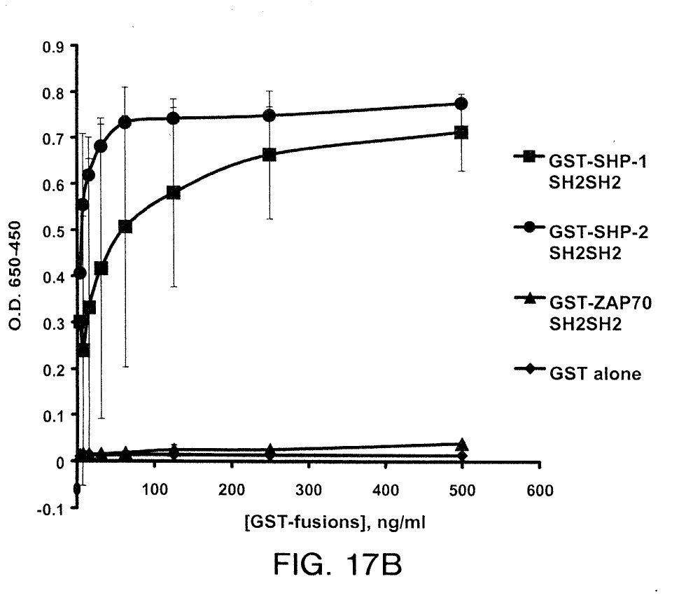

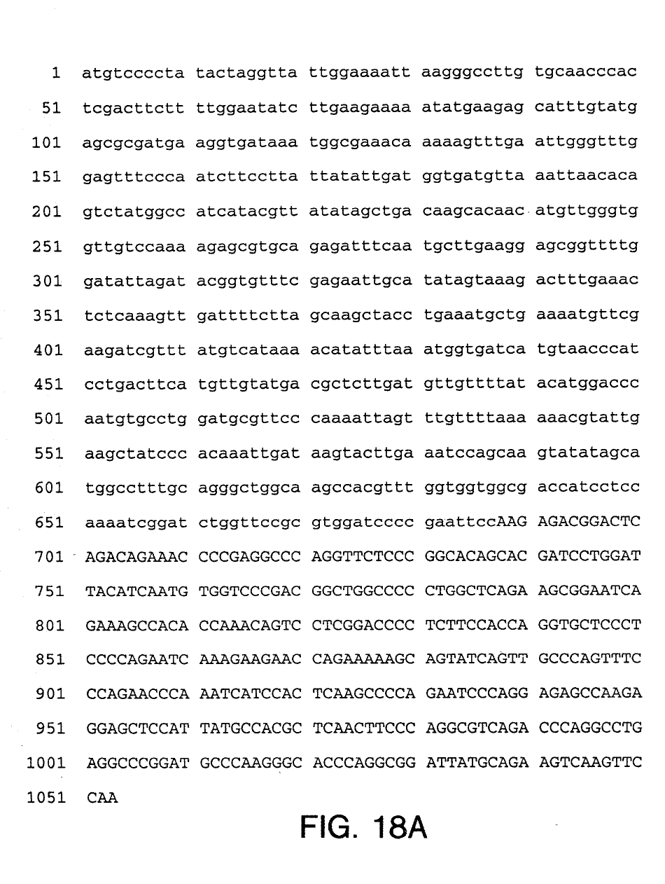

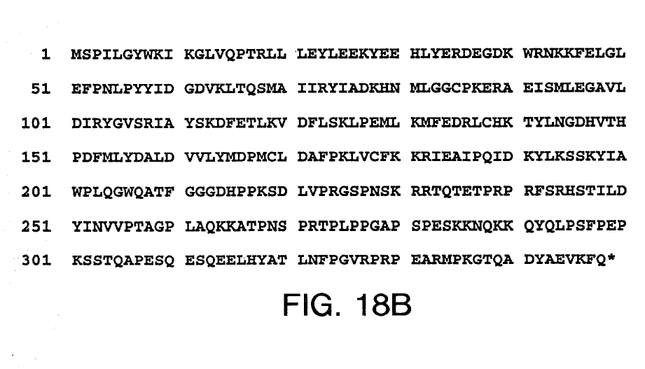

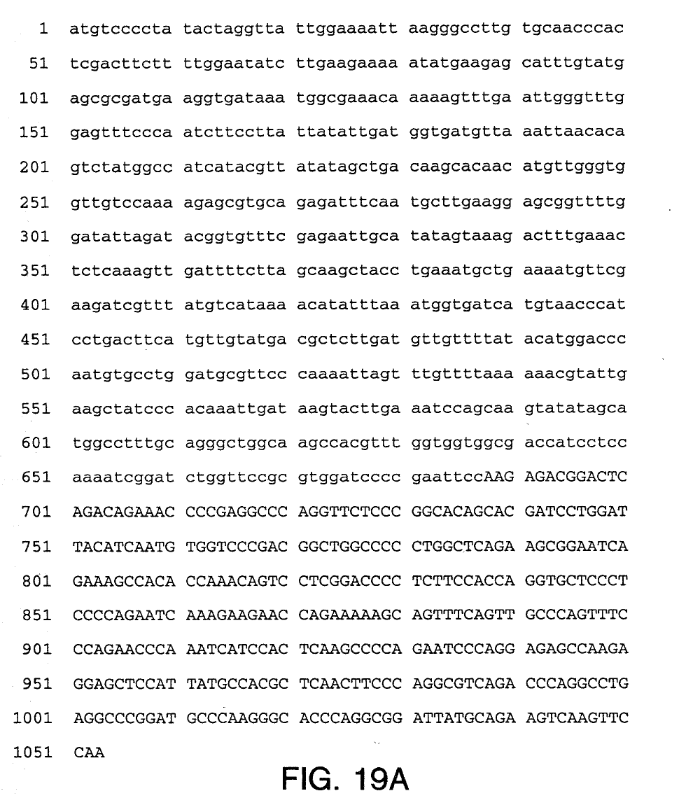

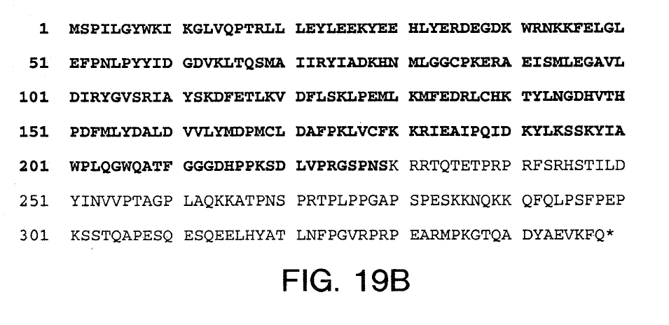

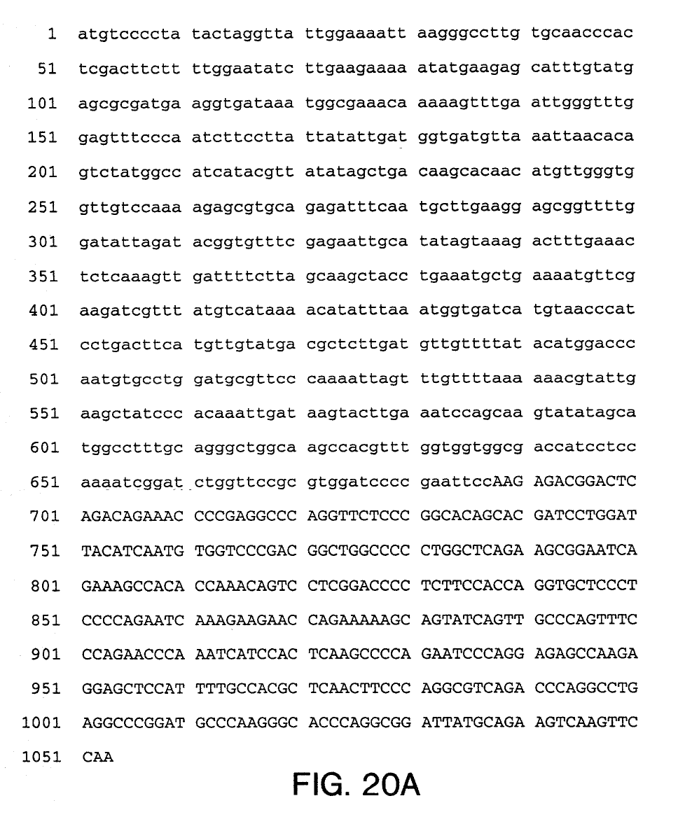

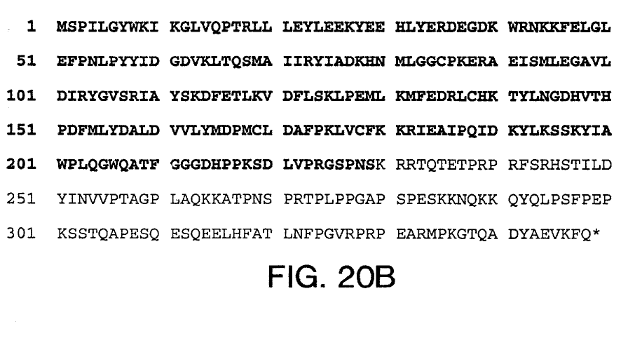

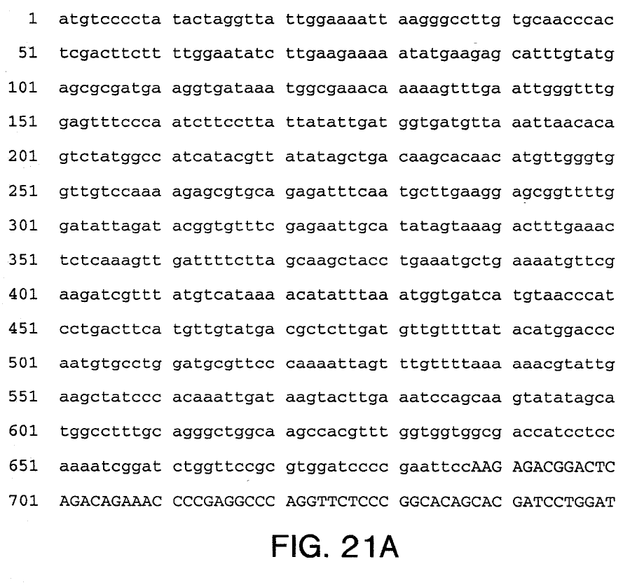

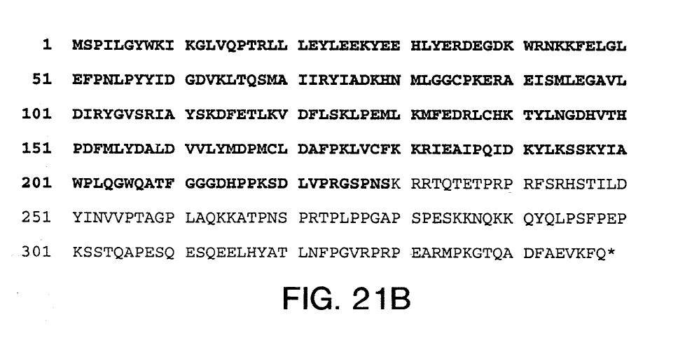

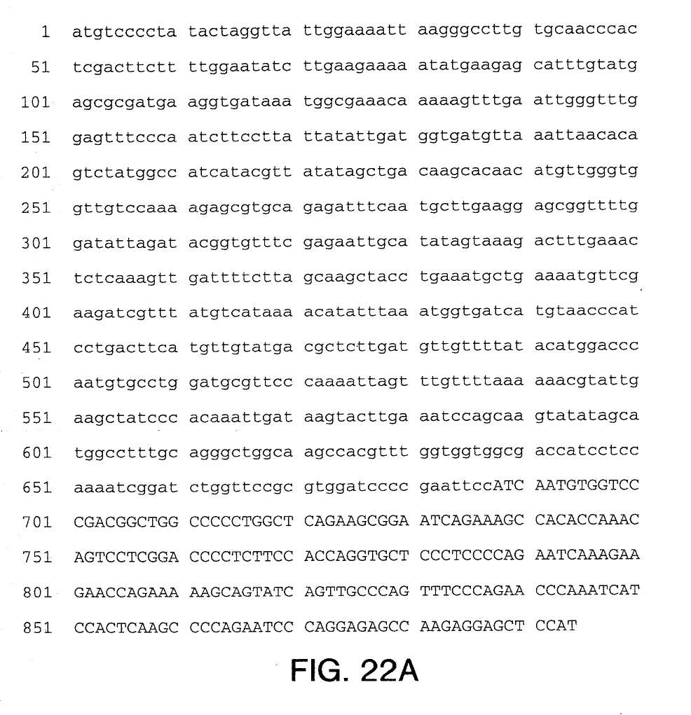

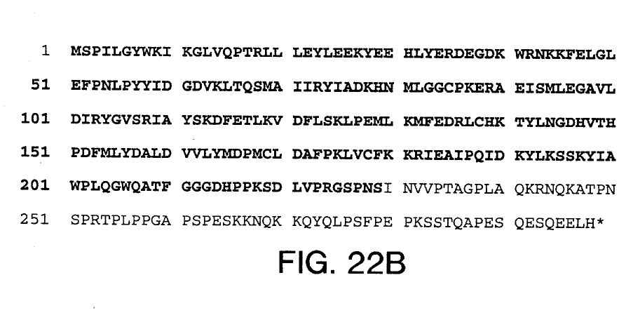

The results, shown in FIG. 17A, indicate that both SHP-1 and SHP-2 from the cell lysates are capable of binding to the cytoplasmic domain—of the SIGLEC L3 protein. The binding of SHP-1, however, was missing from the Y667F mutant, indicating this to be the preferred tyrosine (e.g., single-letter code:Y) for interaction with SHP-1. SHP-2 binding, however, was only diminished by about 50% in the Y667F mutant sample, indicating that SHP-2 may be binding both to the tyrosine at position 667 and to other tyrosines in the cytoplasmic tail of SIGLEC BMSL3.

ITIM Peptide Binding to SHP Proteins by ELISA:

A biotinylated Siglec-10 phosphopeptide (660-678) ESQEELHpYATLNFPGRVPR (ITIM667) was produced by W.M.Keck Biotechnology Resource Center, New Haven Conn. Four μg/ml of phosphopeptide in Blocking Reagent (Hitachi Genetics Systems) was bound to a strepavidin-coated ELISA plate (Pierce, Rockford, Ill.). Plates were washed and then two fold dilutions of the GST fusion proteins, GST alone, GST-SHP-1SH2SH2 or GST-SHP-2SH2SH2 or GST-ZAP-70SH2SH2 were added and incubated for 1 hour at room temperature. Polyclonal anti-GST (prepared in-house by procedures similar to those detailed for Siglec antibody production) was added at 1:1000, HRP-conjugated anti-Rabbit (Biosource at 1:2000 was added and signal detected with peroxidase substrate (KPL, Gaithersburg, Md.).

The results of the cell-free system shown in FIG. 17B, also confirmed that SHP-1 and SHP-2 could both bind with high affinity to a phosphorylated peptide containing the Y667 domain.

### EXAMPLE 14

The following provides a description of the generation of a DNA molecule having the sequence of full-length Siglec-BMS-L3, which encodes the SIGLEC-BMS-L3 protein. The full-length sequence was designated BMSL3-995-2 (FIGS. 6A and B).

The clone designated 652995 (Incyte database), fused to a pSPORT vector (Life Technology/Gibco, Grand Island, N.Y.) includes a complete 3′ end of the Siglec BMS-L3 cDNA. The 652995 clone was digested with restriction enzymes EcoRI and BbrPI and the larger fragment (approximately 6.4 kb) was gel-purified. A second clone, designated 3421048 (Incyte database), included a complete 5′ end of the Siglec BMS-L3 cDNA and was digested with restriction enzymes EcoRI and BbrPI and gel purified (approximately 820 bp). The gel purified fragments were ligated into a pSPORT vector, resulting in a hybrid construct having full-length Siglec BMS-L3 nucleotide sequences and was designated 995-2. The sequence of 995-2 was verified against other SiglecBMS-L3 sequences. The 995-2 clone was digested with restriction enzymes EcoRI and Not I, and ligated into a similarly digested pcDNA3 vector (Invitrogen, Carlsbad, Calif.) for full length expression.

A partial sequence of the 5′ end of the 3421048 was obtained. The sequence is as follows:

| | |
|---|---|
| 5′CAGGCCTGTC TCACGCAGGC CCTGCGCCTC CTATGCGGAG ATGCTACTGC | (SEQ ID NO:69) |
| CACTGCTGCT GTCCTCGCTG CTGGGCGGGT CCCANGCTAT GGATGGGAGA | |
| TTCTGGATAC GAGTGCAGGA GTCAGTGATG GTGCCGGAGG GCCTGTGCAT | |
| CTCTGTGCCC TGCTCTTTCT CCTACCCCCG ACAGGACTGG ACAGGGTCTA | |
| CCCCAGCTTA TGGCTACTGG TTCAAAGCAG TGACTGAGAC A3′ | |

### EXAMPLE 15

The following Example provides a description of Polyacrylamide Glycoconjugate Binding Assays to analyze binding of Siglec-10 to sialic acid.

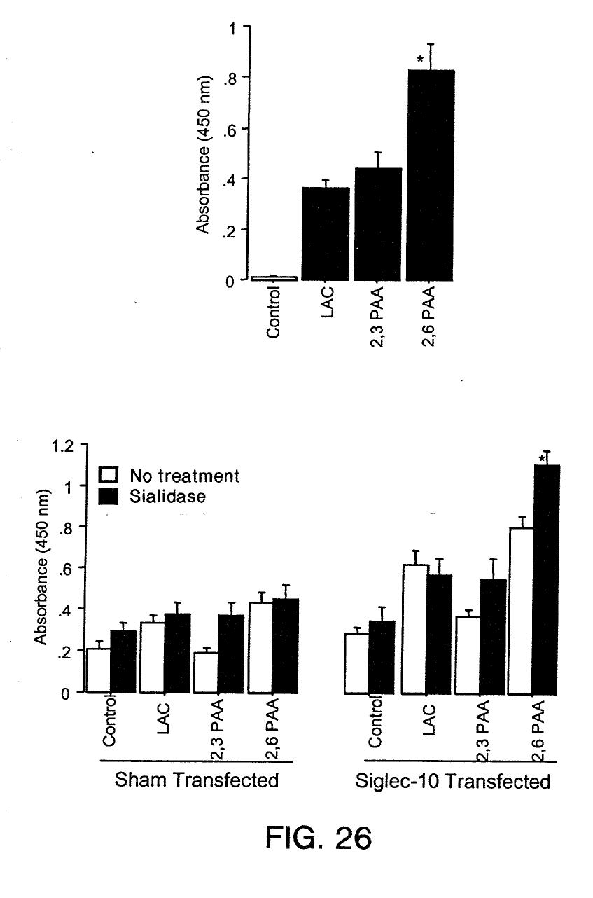

COS7 cells were transiently transfected (see methods above for transfection protocol) with full length Siglec-10 (995-2 in pcDNA3 vector) or sham transfected were plated in 96-well plates within 24 hours of transfection and allowed to attach for 18-22 hours. Half of the plated cells were treated with 0.01 U sialidase (Calbiochem, La Jolla, Calif.) for 1 hour at 37° C. because the treatment has been shown to remove cell surface sialic acids that possibly mask the binding site for other Siglec family members (Zhang et al., 2000). The cells were then washed with DMEM containing 1%BSA and incubated with saturating concentrations (20 μg/ml) of a polyacrylamide polymer containing biotin and carbohydrate (lactose, 3′-sialyllactose or 6′ sialyllactose, GlycoTech Corp., Rockville, Md.). In a parallel cell-free experiment, Immulong plates were coated with purified Siglec-10-hIg fusion protein (200 ng/well) and incubated with 20 μg/ml of the polyacrylamide polymers. After 1 hour, plates were washed and treated with streptavidin-horse radish peroxidase (Vector Labs, Burlingame, Calif.) in DMEM for 30 minutes. After a final wash, TMB peroxidase substrate (KPL, Gaithersburg, Md.) was added and the plates were developed at room temperature. The reaction was stopped with 0.1N HCl and absorbance at 450 nm was determined on a spectrophotometer. The binding preference of Siglec-10 for 2,3′-sialyllactose (2,3′PAA) and 2,6′ sialyllactose (2,6′PAA) was determined by immobilizing Siglec-10-hIg on an Immulon plate and determining the binding of the polyacrylamide biotinylated glycoconjugates (FIG. 26). The 2,6′-PAA conjugate bound significantly greater than either the un-sialylated lactose (negative control) or the 2,3′-PAA. A subsequent cell-based experiment was done to confirm this observation. Full length Siglec-10 (995-2 in pcDNA3) was transfected into COS7 cells by DEAE-dextran method and PAA binding to transfected cells was determined. There was significantly greater binding of the 2,6′-PAA conjugate to transfected COS7 cells following sialidase pretreatment. The need for sialidase treatment suggested that cis-binding of the Siglec-10 could inhibit interaction with the added PAA.

### EXAMPLE 16

The following example provides a description of the generation of monoclonal antibodies to Siglec-10 and utilization of monoclonal antibodies for detection of Siglec-10 protein by FACs analysis and Western blotting

Balb/c mice were immunized with an intraperitoneal injection of Siglec-10-hIg protein in Ribi Adjuvant (Corixa, Hamilton, Mont.) once every 3 weeks. Three days prior to sacrifice, the mice were boosted with an IV injection of Siglec-10-hIg. Splenocytes were aseptically harvested, washed, and mixed 10:1 with mouse myeloma cells (P3x, ATCC, Rockville, Md.) in the presence of PEG 1500 50%(Roche) to induce fusion. Those clones producing antibodies selective for Siglec-10-hIg but not to other hIg, as screened by ELISA, were expanded in roller flasks. The purified monoclonal antibodies were further screened by Western blot of Siglec-10-hIg and other similar fusion proteins. A third screen for antibody specificity was performed using FACs analysis of COS7 cells that were transfected with full length Siglec-10 expression construct.

Siglec Protein Expression.

FACs analysis of peripheral blood cell populations and cell lines was performed to determine surface protein expression (Table 2). Anti-Siglec-10 antibody bound to isolated granulocytes (eosinophils and neutrophils) and CD 14+ monocytes with large shifts in fluorescence intensity. The antibody did not bind to other blood cells including CD28+ cells and CD3+ cells.

Table 2 shows expression of Siglec-10 on hematopoietic cell lines and primary leukocytes. Biotinylated monoclonal anti-Siglec-10 was added to cells followed by treatment with FITC-conjugated streptavidin. The antibody was chosen based on immunoreactivity to COS7 cells transfected with Siglec-10 as determined by FACs. For peripheral blood mononuclear cell preparations (PBMC), a secondary PE-conjugated antibody was used to distinguish sub-populations. The percentage of total cells with increased fluorescence is indicated. Data shown represents the mean of 2-3 experiments.

TABLE 2

| | | FITC | Anti- |
|---|---|---|---|
| Cell line | Type | alone (%) | Siglec-10 (%) |
| Ramos | B-cell (lymphoma) | 0.5 | 89.9 |
| THP-1 | Monocyte (lymphoma) | 1.7 | 39.1 |
| Jurkat | T-cell (lymphoma) | 0.4 | 76.6 |
| U973 | Monocyte (leukemia) | 2.0 | 33.6 |
| HL60 | Monocyte (leukemia) | 0.6 | 93.3 |
| K562 | Monocyte (leukemia) | 0.5 | 96.2 |
| COS7 | Naive | 2.4 | 15.4 |
| COS7 | Siglec-10 Transfected | 4.7 | 63.3 |
| Blood | | Siglec- | FITC+ and |
| Population | PE+ (%) | FITC+ (%) | PE+ (%) |
| PBMC | | 19.9 | |
| CD20+ | 8.3 | | 2.4 |
| CD14+ | 12.1 | | 12.0 |
| CD4lo+ | 11.7 | | 12.0 |
| CD4hi+ | 63.0 | | 0 |
| CD3+ | 65.0 | | 0 |
| CD28+ | 47.5 | | 2.0 |
| Granulocytes | | 88.6 | |

Western Blotting for SIGLEC-10.

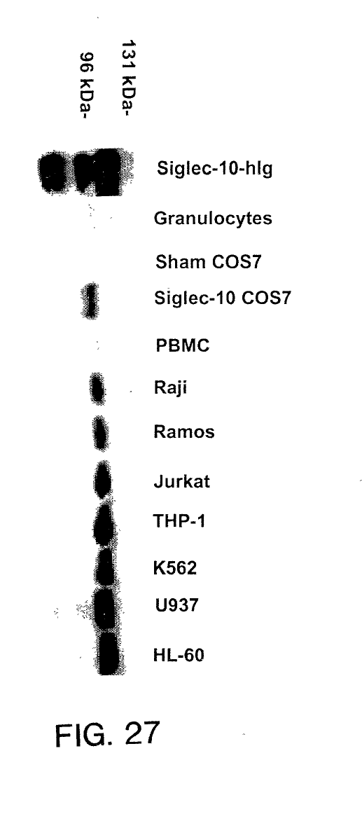

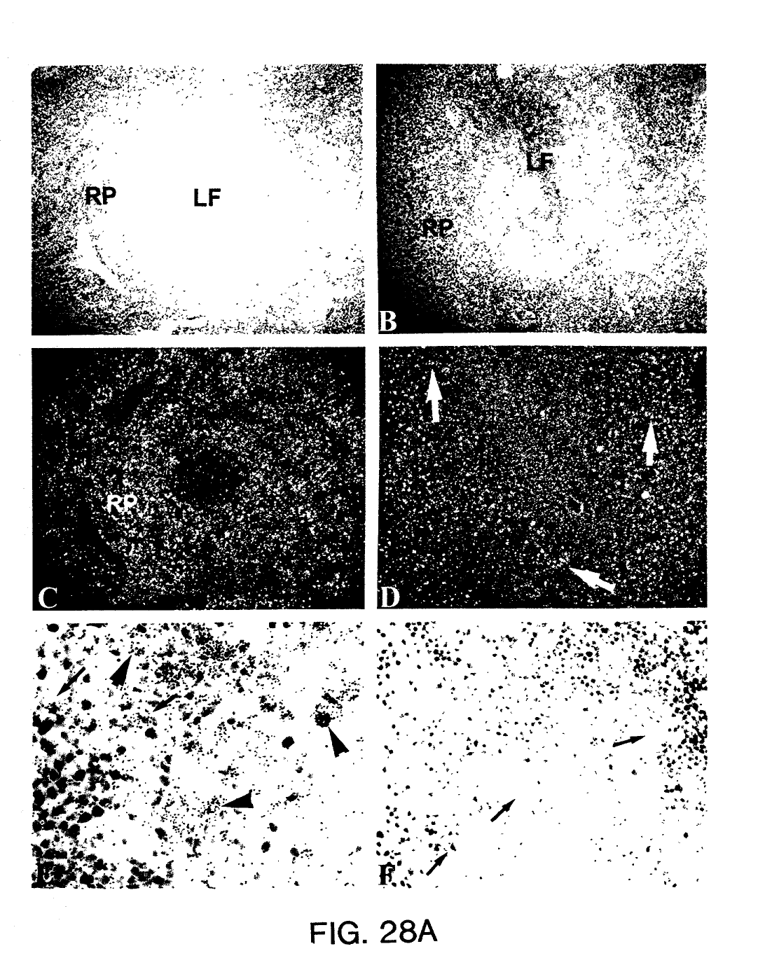

Ten micrograms of cell lysates (Triton-X-100-soluble protein fraction) from several cell lines and peripheral blood cell preparations were mixed with sample buffer and resolved by SDS-PAGE (4-20% gradient gel) and transferred to nitrocellulose by standard western blotting techniques. The blots were then stained with anti-Siglec-10 monoclonal antibody followed by an HRP-conjugated secondary antibody (goat anti-mouse, Biosource Int., Camarillo Calif.). Stained proteins were imaged by adding a chemiluminescent detection reagent (Renaissance, NEN Bio Products, Boston, Mass.) using a Phosphorlmager 445 SI (Molecular Dynamics, Sunnyvale, Calif.). The anti-Siglec-10 mAb recognized a single band with a molecular mass of approximately 76 kDa (FIG. 27). There were no other visible bands, implying that the antibody is specific for Siglec-10. Granulocytes and several blood cell lines appear to express Siglec-10 (FIG. 27).

### EXAMPLE 17

The following example provides a description of detection of Siglec-10 positive hybridization signals in non-human primates (NHP) and human tissues using in situ hybridization.

Human ³⁵S labeled RNA probes (riboprobes) for Siglec 10 were created via in vitro transcription utilizing PCR product templates. Gene specific primer (GSP) sets were obtained from Life Technologies (Rockville, Md.) according to probe primer sequence data (Siglec Manuscript, IIPD) for Siglec 10 L3 probe (5′ (724-744) TGCTCAGCTTCACGCCCAGAC; 3′ (1447-1456) TGCACGGAGAGGCTGAGA GA). Amplicons were obtained from PCR amplification of full length Siglec 10 gene cloned into a pSport plasmid vector. Gel electrophoresis was run and correct size bands were cut from gel. These bands were purified (Gel SNAP Purification kit, Invitrogen, Carlsbad, Calif.) and then subcloned utilizing the TOPO-TA cloning kit (Invitrogen, Carlsbad, Calif.) into the pCRII vector. Miniprep and sequence analyses combined with GenBank Blast were used to confirm the sequence identities (GenBank confirmed 100% 274 bp identity to Chromosome 19. Separate Blast2 of novel Siglec 10 sequence and miniprep sequence results gave 100% homology). The minipreps were then PCR amplified using GSP and T7 and/or Sp6 primers. Riboprobes were produced utilizing the RNA polymerases Sp⁶ and T7 and a commercially available kit (Riboprobe® Combination System, Promega, Madison, Wis.), and in situ hybridization was performed.

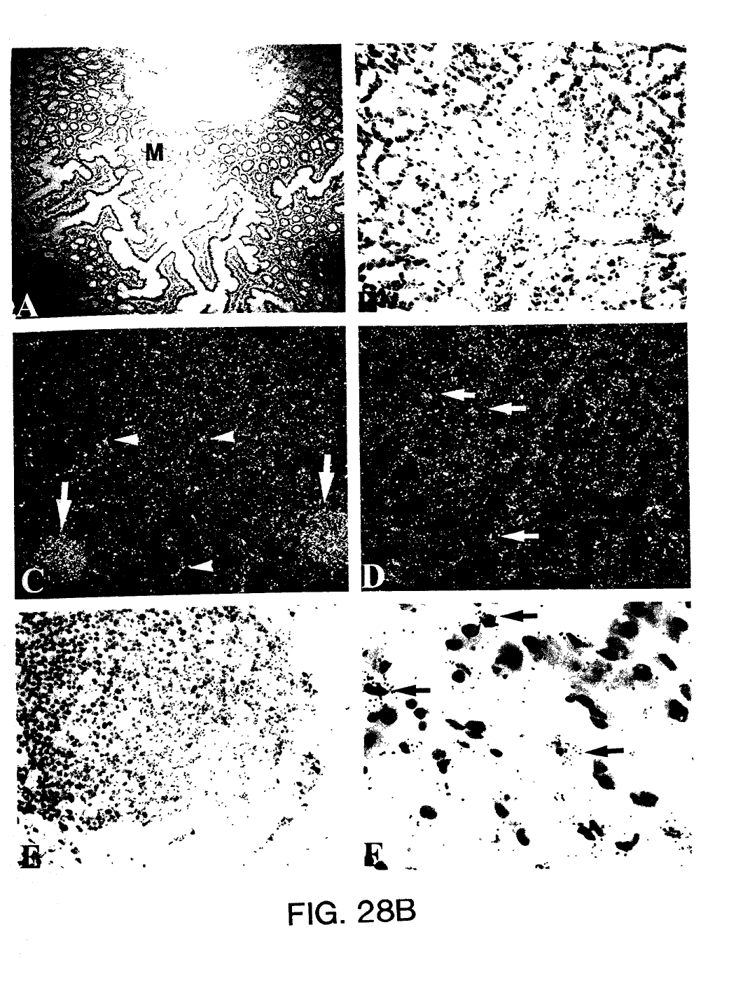

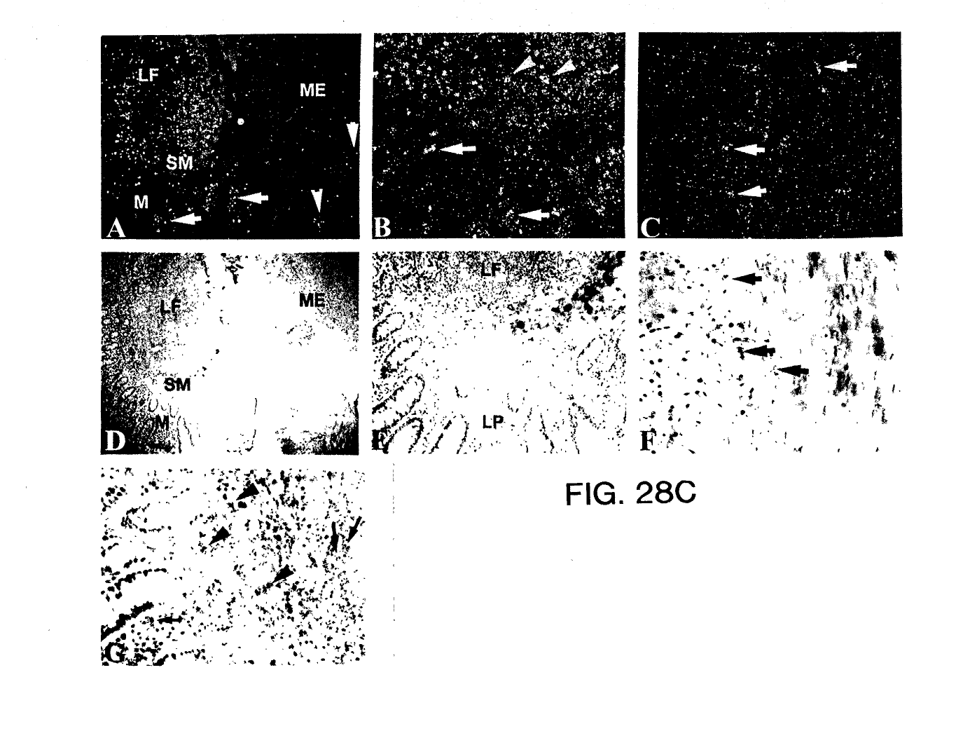

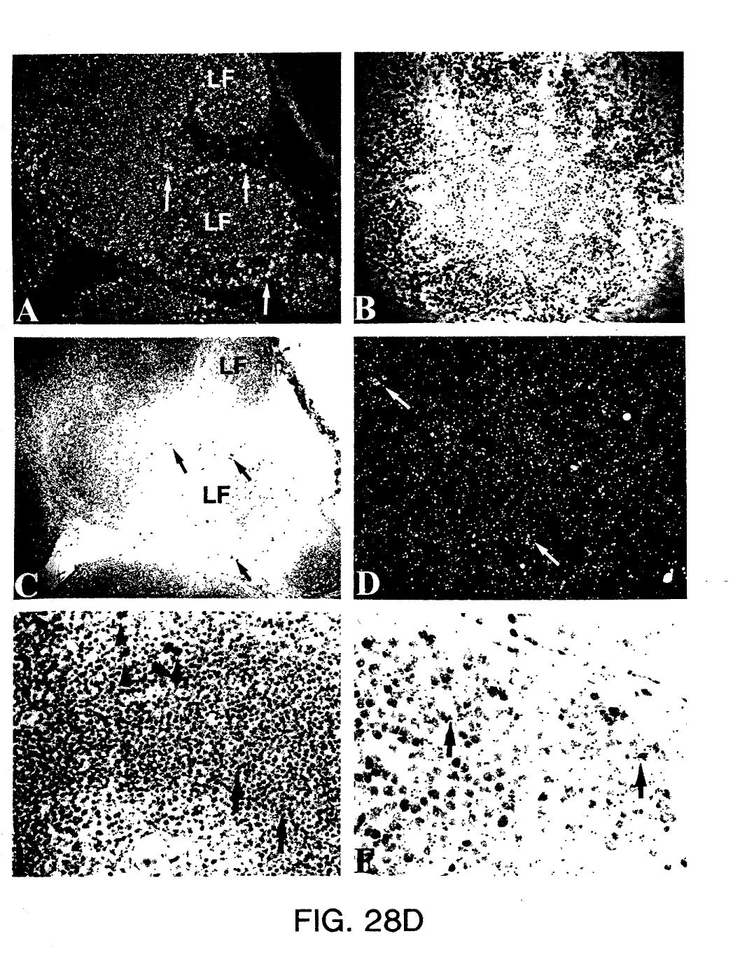

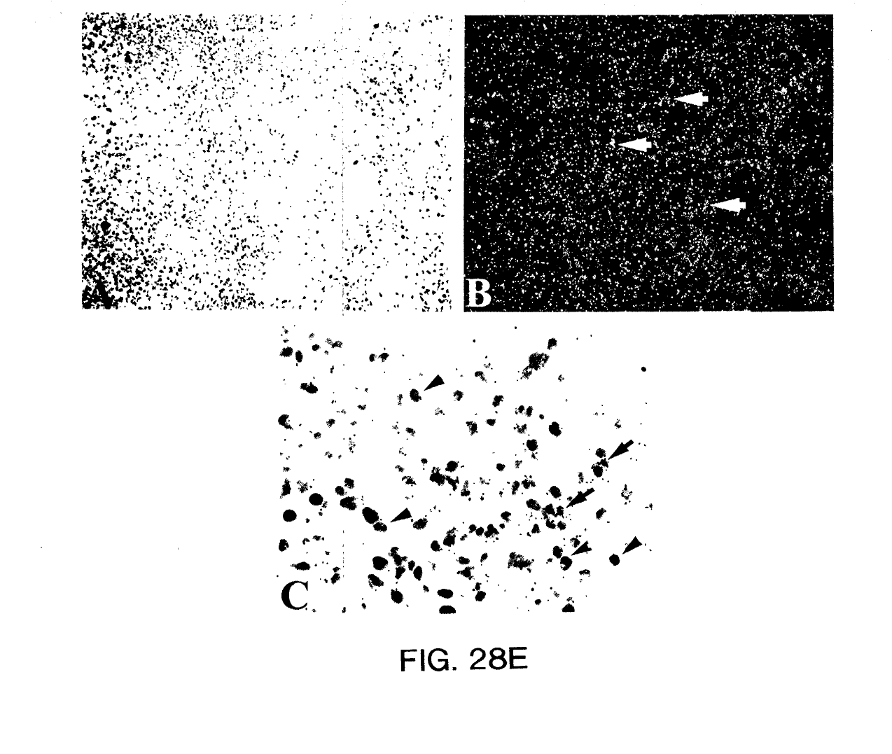

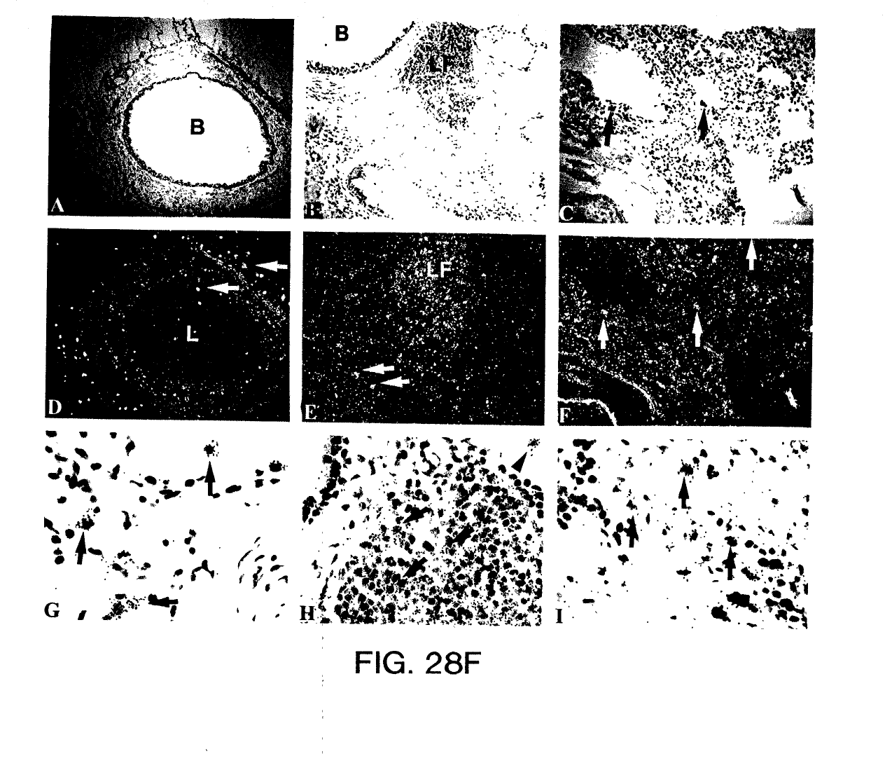

FIG. 28 shows micrograph composite images of in situ hybridization detailing the distribution of Siglec-10 positive hybridization signals in non-human primate and human tissues.

FIG. 28a shows Siglec-10 positive hybridization in non-human primate (NHP) (Panels A, C, E)/human spleen (Panels B, D, F). Panel A (Brightfield, 40× magnification) shows Lymphoid follicle (LF) and surrounding red pulp (RP) area, NHP spleen. Panel B (Brightfield, 40× magnification) shows Lymphoid follicle (LF) and surrounding red pulp area (RP), Human spleen. Pnael C (40× magnification) is Darkfield of Panel A showing Siglec 10 hybridization signals associated with the red pulp area (RP). Panel D (40× magnification) is Darkfield of Panel B showing Siglec-10 hybridization signals (white foci) in the red pulp area (arrows). Panel E (Brightfield, 200X magnification) shows detail of red pulp showing Siglec 10 hybridization signal (black foci) associated with lymphocytes (arrows) and macrophages (arrowheads), NHP. Panel F (Brightfield, 200× magnification) shows detail of red pulp showing Siglec-10 hybridization signals (black foci) associated with macrophages (arrows), Human.

FIG. 28b shows Siglec-10 positive hybridization in NHP Jejunum (panels A, C, E); Human liver (B, D, F). Panel A (Brightfield, 40× magnification) shows Mucosa (M) with lymphoid follicles (LF), NHP jejunum. Panel B (Brightfield, 40× magnification) shows Liver, Human. Panel C shows darkfield of Panel A showing Siglec 10 hybridization signals in lymphoid follicles (arrows) and foci in lamina propria of mucosa (arrowheads). Panel D shows darkfield of Panel B showing Siglec-10 hybridization signals along sinusoids (arrows). Panel E (Brightfield, 200× magnification) shows detail of Siglec-10 hybridization signals associated with lymphocytes in lymphoid follicles of mucosa, NHP jejunum. Panel F (Brightfield, 400× magnification) shows detail of Siglec 10 hybridization signals (arrows) associated with Kupffer cells (resident macrophages), Human liver.

FIG. 28c shows Siglec-10 positive hybridization in Non-human Primate Colon. Panel A (Darkfield, 40× magnification) shows transverse section of colon with mucosa (M), submucosa (SM), muscularis externa (ME) and lymphoid follicle (LF) in submucosa. Siglec 10 hybridization signal is present in the lamina propria of mucosa (arrows), LF of submucosa (LF), and multifocally in the interstitium of muscularis extema (arrowheads). Panel B (Darkfield, 100× magnification) shows detail of Panel A showing Siglec 10 hybridization signals in mucosal lamina propria (arrows) and submucosal LF (arrowheads). Panel C (Darkfield, 100× magnification) shows detail of Panel A showing Siglec 10 hybridization signals in the interstitium of the muscularis externa (arrows). Pannel D is Brightfield of Panel A showing mucosa (M), submucosa (SM) with lymphoid follicle (LF), and muscularis extema (ME). Panel D is Brightfield of Panel B showing mucosal lamina propria (LP) and lymphoid follicle (LF) in submucosa. Panel E is Brightfield of Panel C showing Siglec 10 positive mononuclear cells (arrows) in the interstitium of the muscularis extema. Panel F (Brightfield, 200× magnification) shows detail of lamina propria of the mucosa showing Siglec 10 hybridization signals associated with lymphocytes (arrows) and macrophages (arrowheads).

FIG. 28d shows distribution of Siglec-10 positive hybridization signal in NHP (panels (A, C, E)/Human Lymph Node (panels B, D, F). Panel A (Darkfield, 10× magnification) shows Siglec 10 hybridization signals associated with lymphoid follicles (LF). Prominent cells are melanomacrophages (arrows), NHP lymph node. Panel B (Brightfield, 40× magnification) shows a weak Siglec 10 hybridization signals associated with lymphocytes, Human lymph node. Panel C is a Brightfield of Panel A showing LF and melanomacrophages (arrows). Panel D is a Darkfield of Panel B showing weak Siglec 10 signal (arrow). Panel E (Brightfield, 200× magnification) D depicts detail of LF showing Siglec 10 hybridization signals associated with lymphocytes (arrows) and melanomacrophages (arrowheads). Panel F (Brightfield, 400× magnification) depicts detail of LF showing weak Siglec 10 hybridization signals associated with lymphocytes (arrows).

FIG. 28e shows distribution of Siglec-10 positive hybridization signals Siglec-10 RNA Asthma Lung. Panel A (Brightfield, 100×.) shows Lung parenchyma infiltrated by a mixed inflammatory cell population, which includes eosinophils, macrophages, and lymphocytes, Human lung. Panel B is Darkfield of Panel A showing multifocal Siglec 10 hybridization signals (arrows). Panel C (Brightfield, 400× magnification) depicts detail of inflammatory cells of lung showing Siglec-10 hybridization signals associated with macrophages (arrows), but no signal associated with eosinophils (arrowheads).

FIG. 28f shows binding of Siglec-10 RNA to Non-human Primate (Panels A, B, D, E, G, H)/Human Lung (Panels C, F, I). Panel A (Brightfield, 40× magnification) shows Airway bronchiole (B), NHP, lung. Panel B (Brightfield, 100× magnification) shows detail of lymphoid follicle (LF) in subbronchial area, Bronchiole (B), NHP lung. Panel C (Brightfield, 100× magnification) shows Lung parenchyma with brown-stained alveolar macrophages (anthrosilicosis) (arrows), Human lung. Panel D is Darkfield of Panel A showing Siglec-10 hybridization signals (arrows) in airway lumen (L) and lung parenchyma (arrowheads). Panel E is Darkfield of Panel B showing Siglec-10 hybridization signal in LF and in lung parenchyma (arrows). Panel F is Darkfield of Panel C showing Siglec-10 hybridization signals associated with alveolar macrophages (arrows). Panel G (Brightfield, 400× magnification) depicts Detail of Panel A showing Siglec-10 hybridization signals associated with alveolar macrophages (arrows). Panel H (Brightfield, 400×) depicts Detail of Panel B showing Siglec 10 hybridization signals associated with lymphocytes of the LF (arrows) and an alveolar macrophage (arrowhead). Panel I (Brightfield, 400× magnification.) depicts Detail of Panel C showing Siglec-10 hybridization signals associated with brown-stained (anthrosilicosis) alveolar macrophages (arrows).

The final results of in situ hybridization are described in Table 3.

TABLE 3
Siglec 10 ISH scores for select human and non-human primate tissues

| | Human | | Non-human primate | |
|---|---|---|---|---|
| | Score¹ | Tissue | Score | Tissue |
| Colon normal | + | Lymphoid follicles (luminal aspect | + + + + | Lymphoid follicles, (luminal aspect of |
| | | of lamina propria) | | lamina propria); |
| | | | + + | TBD cells within the muscularis |
| | | | externa | |
| Colon IBD | + | Lymphoid follicles (luminal aspect | NE² | |
| | | of lamina propria) | | |
| Ileum | + | Weak lymphoid follicles, rare | + + | Lymphoid follicles, GALT³ |
| | | lamina propria cells | | |
| Jejunum | − | | + + + | Lymphoid follicles, GALT |
| Stomach | + | Weak lymphoid follicles, rare | + + | Lymphoid follicles, TBD cells of |
| | | lamina propria cells | | gastric pits |
| Duodenum | − | | + + | Lymphoid follicle, lamina propria of |
| | | | | mucosa/sub-mucosa |
| | | | + | Villus enterocytes |
| Lymph node | + | Rare lymphocyte | + + + | Lymphocyte subpopulation |
| | −? | macrophage | | Dendritic cells/melano-macrophages |
| Liver | + + | Kupffer cells | NE | |
| *Cartilage (DJD) | * | | NE | |
| Lung - normal | + + | macrophages | + + + | Alveolar macrophages |
| | | | + + | Lymphoid follicle lymphocytes |
| | | | | Airway epithelium with lymphocyte |
| | | | + | infiltration |
| Lung - asthma | + + | Inflammatory foci | NE | |
| | − | eosinophils | | |
| Spleen | + + + | Macrophages | + + + + | Red pulp very strong |
| | | Lymphocytes | + + + | Regions of lymphoid follicle |

¹ISH score: no signal, −; minimal hybridization signal, +; mild signal, + +; moderate signal, + + +; marked signal, + + + +
²NE, not examined
³GALT, gut-associated lymphoid tissue in lamina propria
*cartilage had a large amount of background and tissue could not be read for either − or + hybridization

Various publications are cited herein that are hereby incorporated by reference in their entirety.

As will be apparent to those skilled in the art to which the invention pertains, the present invention may be embodied in forms other than those specifically disclosed above without departing from the spirit or essential characteristics of the invention. The particular embodiments of the invention described above, are, therefore, to be considered as illustrative and not restrictive. The scope of the present invention is as set forth in the appended claims rather than being limited to the examples contained in the foregoing description.

What is claimed is:

1. An isolated SIGLEC protein comprising an amino acid sequence beginning with Ala141 and ending with Ser198 as shown in FIG. 6B.

2. An isolated SIGLEC protein comprising an amino acid sequence beginning with Ala141 and ending with Ser198 as shown in FIG. 6B and is encoded by a nucleic acid molecule that hybridizes, under stringent conditions to a nucleic acid molecule that is complementary to the nucleic acid as shown in any one of FIGS. 2A, 3A, 4A, 5A, 6A, 7A, 8A, and 9A.

3. The isolated SIGLEC protein of claim 1 or 2 comprising the amino acid sequences as shown in any one of FIGS. 4B, 5B, and 6B.

4. An isolated SIGLEC protein comprising an amino acid sequence that is encoded by a nucleic acid molecule that hybridizes, under stringent conditions to a nucleic acid molecule that is complementary to the nucleic acid as shown in any one of FIGS. 2A, 3A, 4A, 5A, 6A, 7A, 8A, and 9A.

5. An isolated SIGLEC protein of claim 4 having an amino acid sequence as shown in any of FIGS. 2B, 3B, 4B, 5B, 6B, 7B, 8B.

6. A peptide fragment of the protein of claim 3.

7. A peptide fragment of claim 6 having an amino acid sequence beginning with Ala14l and ending with Ser198 as shown in FIG. 6B, or any fragment thereof.

8. A peptide fragment of claim 6 comprising the cytoplasmic domain having an amino acid sequence beginning with Lys576 and ending with Gln697 as shown in FIG. 6B or any fragment thereof.

9. A mutant SIGLEC BMS protein comprising a cytoplasmic domain, wherein at least one tyrosine in the cytoplasmic domain is substituted with an amino acid selected from the group consisting of phenylalanine, leucine, tryptophan, and threonine.

10. The mutant SIGLEC BMS protein of claim 9 having the amino acids shown in FIG. 6b, wherein the tyrosine in the cytoplasmic domain is any of the tyrosines at position 597, 641, 667, or 691.

11. An isolated Siglec nucleic acid molecule comprising a nucleic acid beginning with codon GCC at position +421 and ending at codon TCA at position +594 as shown in FIG. 6A, wherein the nucleic acid hybridizes, under stringent conditions to a nucleic acid molecule that is complementary to the nucleic acid as shown in any one of FIGS. 2A, 3A, 4A, 5A, 6A, 7A, 8A, and 9A.

12. An isolated Siglec nucleic acid molecule that encodes the protein of claim 1, 2, or 4.

13. The isolated Siglec nucleic acid molecule of claim 12, comprising the sequence shown in any of (a) FIG. 2A beginning at codon GGC at position +12 and ending at codon CCA at position +1760; (b) FIG. 3A beginning at codon GAT at position +3 and ending at codon CAA at position +1868; (c) FIG. 4A beginning at codon GGA at position +12 and ending at codon CAA at position +1736; (d) FIG. 5A beginning at codon CCC at position +2 and ending at codon ATG at position +1291; (e) FIG. 6A beginning at codon ATG at position +1 and ending at codon CAA at position +2091; (f) FIG. 7A beginning at codon CTG at position +1 and ending at codon GGC at position +1398; (g) FIG. 8A beginning at codon ATG at position +43 and ending at codon AGA at position +1431; or (h) FIG. 9A beginning at codon ATG at position +57 and ending at codon AGT at position +914.

14. The isolated Siglec nucleic acid molecule of claim 11, or 13, which is DNA or RNA.

15. An isolated nucleic acid molecule which is complementary to the nucleotide sequence of the molecule of claim 11, or 13.

16. A vector comprising the isolated nucleic acid molecule of claim 11, or 13.

17. A host-vector system comprising the vector of claim 16, in a suitable host cell.

18. A method for producing a SIGLEC-BMS protein having the amino acid sequence of any of FIGS. 2B, 3B, 4B, 5B, 6B, 7B, 8B, and 9B, comprising:
a) culturing the host-vector system of claim 17 under suitable conditions so as to produce the protein; and
b) recovering the protein so produced.

19. A SIGLEC-BMS protein produced by the method of claim 18.

20. A chimeric protein comprising the polypeptide of claim 1, 2, or 4, or a fragment thereof, fused to a heterologous polypeptide.

21. A chimeric protein comprising an extracellular domain of the polypeptide of claim 1, 2, or 4, fused to a heterologous polypeptide.

22. A chimeric protein comprising the cytoplasmic domain of the polypeptide of claim 1, 2, or 4, fused to a heterologous polypeptide.

23. The chimeric protein of claim 20, 21, or 22, wherein the heterologous polypeptide is an immunoglobulin constant region.

24. The chimeric fusion protein of claim 20, 21, or 22, wherein the heterologous protein is Glutathione S-transferase.

25. An antibody or antibody fragment having an antigen binding site, wherein the antigen binding site specifically recognizes and binds the protein of claim 1, 2 or 4.

26. The antibody of claim 25, wherein the antibody is a polyclonal antibody or a monoclonal antibody.

27. The antibody of claim 26, wherein the monoclonal antibody is designated SIGLEC-10-9, SIGLEC-10-13, SIGLEC-10-14, SIGLEC-10-27, or SIGLEC-10-61, and which are collectively deposited as ATCC Accession No (______).

28. The antibody of claim 25, wherein the antibody is a chimeric antibody having a murine antigen-binding site and a humanized region that regulates effector functions.

29. A method for identifying a test molecule that modulates an immune response induced by Siglec-10 positive cells comprising:
a. contacting Siglec-10 positive cells with the test molecule; and
b. determining whether the immune response is modulated.

30. The method of claim 29, wherein the test molecule that modulates an immune response targets an extracellular domain of a Siglec-10 on Siglec-10 positive cells.

31. The method of claim 30, wherein the extracellular domain encompasses at least one of the Ig-like domains of a Siglec-10.

32. The method of claim 31, wherein the Ig-like domain of Siglec-10 ia an Ig (V) domain of a Siglec-10.

33. The method of claim 31, wherein the Ig-like domain of Siglec-10 is an Ig (C) domain of a Siglec-10.

34. A method for modulating an immune response induced by Siglec-10 positive cells comprising contacting Siglec-10 positive cells with a monoclonal antibody directed against Siglec-10 under suitable conditions so that the immune response is modulated.

35. The method of claim 34, wherein the antibody targets an extracellular domain of a Siglec-10.

36. The method of claim 35, wherein the extracellular domain so targeted is an Ig-like domain of a Siglec-10.

37. The method of claim 36, wherein the Ig-like domain is an Ig (V) domain of a Siglec-10.

38. The method of claim 36, wherein the Ig-like domain is an Ig (C) domain of a Siglec-10.
